SEQUENCE LISTING (34 sequences)

SEQ ID NO: 1 - protein, 10 aa, Artificial
  kinase sensor
GRTGRRFSXP

SEQ ID NO: 2 - protein, 10 aa, Artificial
  kinase sensor
GRTGRRXSYP

SEQ ID NO: 3 - protein, 10 aa, Artificial
  kinase sensor
GRTXRRFSYP

SEQ ID NO: 4 - protein, 10 aa, Artificial
  kinase sensor
GRXGRRFSYP

SEQ ID NO: 5 - protein, 10 aa, Artificial
  kinase sensor
XRTGRRFSYP

SEQ ID NO: 6 - protein, 11 aa, Artificial
  kinase sensor
XGRTGRRFSYP

SEQ ID NO: 7 - protein, 11 aa, Artificial
  kinase sensor
XGRTGRRFSYP

SEQ ID NO: 8 - protein, 11 aa, Artificial
  kinase sensor
XGRTGRRFSYP

SEQ ID NO: 9 - protein, 11 aa, Artificial
  kinase sensor
XGRTGRRFSYP

SEQ ID NO: 10 - protein, 11 aa, Artificial
  kinase sensor
XGRTGRRFSYP

SEQ ID NO: 11 - protein, 11 aa, Artificial
  kinase sensor
XGRTGRRFSYP

SEQ ID NO: 12 - protein, 11 aa, Artificial
  kinase sensor
XGRTGRRFSYP

SEQ ID NO: 13 - protein, 10 aa, Artificial
  kinase sensor
GRTGRRFSXP

SEQ ID NO: 14 - protein, 10 aa, Artificial
  kinase sensor
GRTGRRXSYP

SEQ ID NO: 15 - protein, 10 aa, Artificial
  kinase sensor
GRTXRRFSYP

SEQ ID NO: 16 - protein, 10 aa, Artificial
  kinase sensor
GRXGRRFSYP

SEQ ID NO: 17 - protein, 10 aa, Artificial
  kinase sensor
XRTGRRFSYP

SEQ ID NO: 18 - protein, 10 aa, Artificial
  PKA substrate
GRTGRRFSYP

SEQ ID NO: 19 - protein, 10 aa, Artificial
  PKA substrate
GRTGRRFSYP

SEQ ID NO: 20 - protein, 10 aa, Artificial
  kinase sensor
XRTGRRFXYP

SEQ ID NO: 21 - protein, 11 aa, Artificial
  kinase sensor
GXTGRRXSLPA

SEQ ID NO: 22 - protein, 11 aa, Artificial
  kinase sensor
GXTGRRXSLPA

SEQ ID NO: 23 - protein, 11 aa, Artificial
  kinase sensor
GXTGRRXSLPA

SEQ ID NO: 24 - protein, 11 aa, Artificial
  kinase sensor
GRTGRRXSLPK

SEQ ID NO: 25 - protein, 11 aa, Artificial
  kinase sensor
GRTGRRXSLPK

SEQ ID NO: 26 - protein, 11 aa, Artificial
  kinase sensor
GRTGRRXSLPK

SEQ ID NO: 27 - protein, 11 aa, Artificial
  kinase sensor
GRTGRRXSLPK

SEQ ID NO: 28 - protein, 11 aa, Artificial
  kinase sensor
GRTGRRXSLPK

SEQ ID NO: 29 - protein, 11 aa, Artificial
  kinase sensor
GRTGRRXSLPK

SEQ ID NO: 30 - protein, 11 aa, Artificial
  kinase sensor
GRTGRRXSLPK

SEQ ID NO: 31 - protein, 10 aa, Artificial
  kinase sensor
GRTGRRXSYP

SEQ ID NO: 32 - protein, 10 aa, Artificial
  kinase sensor
GRTGRRXSYP

SEQ ID NO: 33 - protein, 10 aa, Artificial
  kinase sensor
XRTGRRFSYP

SEQ ID NO: 34 - protein, 10 aa, Artificial
  kinase sensor
XRTGRRFSYP

(19) United States
(12) Patent Application Publication
(10) Pub. No.: US 2008/0318246 A1
(43) Pub. Date: Dec. 25, 2008

(54) Deeply quenched enzyme sensors

(75) Inventors: David S. Lawrence, Chapel Hill, NC (US); Vyas Sharma, Chapel Hill, NC (US); Richard S. Agnes, Cleveland Heights, OH (US)

Correspondence Address: QUINE INTELLECTUAL PROPERTY LAW GROUP, P.C., P O BOX 458, ALAMEDA, CA, 94501, US

(73) Assignee: The Albert Einstein College of Medicine of Yeshiva University

(21) Appl. No.: 12/075,118

(22) Filed: Mar. 6, 2008

Related U.S. Application Data
(60) Provisional application No. 60/905,718, filed on Mar. 7, 2007.

(51) Int. Cl. G01N 33/53; C12Q 1/00; C12Q 1/48; C07K 2/00; C12Q 1/02; C12Q 1/42

(52) U.S. Cl. 435/7.1; 435/4; 435/15; 435/21; 435/29; 530/300

(57) ABSTRACT

Sensors for detecting enzyme activity are provided that include a substrate module comprising a substrate for the enzyme of interest and a fluorescent label, a quencher, and a detection module. The detection module binds to the substrate module either before or after the enzyme acts on the substrate and sequesters the label from the quencher, resulting in an increased signal from the label. Sensors for detecting enzyme activity are also provided that include a substrate for the enzyme, a label, and a quencher that quenches the label. Action of the enzyme on the substrate results in a conformational change that relieves quenching. Sensors for detecting protein-protein interactions are also provided that include a quencher and a labeled first polypeptide. Binding of the first polypeptide to a second polypeptide sequesters the label from the quencher, resulting in an increased signal from the label. Methods using the sensors to detect enzyme activity and to screen for compounds affecting enzyme activity or to detect protein-protein interactions and to screen for compounds affecting protein-protein interactions, respectively, are also described.

### CROSS-REFERENCE TO RELATED APPLICATIONS

This application is a non-provisional utility patent application claiming priority to and benefit of the following prior provisional patent application: U.S. Ser. No. 60/905,718, filed Mar. 7, 2007, entitled “DEEPLY QUENCHED ENZYME SENSORS AND BINDING SENSORS” by David S. Lawrence et al., which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant Nos. GM067198 and NS048406 from the National Institutes of Health. The government may have certain rights to this invention.

### FIELD OF THE INVENTION

The invention relates to sensors for detecting enzyme activity, and uses thereof, where the enzyme sensors include a substrate module comprising a substrate for the enzyme, a label, and a quencher, and a detection module. Binding of the substrate module to the detection module can sequester the label from the quencher, resulting in an increase in signal from the label. The invention also relates to sensors for detecting enzyme activity, and uses thereof, where the enzyme sensors include a substrate for the enzyme, a label, and a quencher that quenches the label. Action of the enzyme on the substrate results in a conformational change that relieves quenching.

### BACKGROUND OF THE INVENTION

Detection of enzyme activity is a necessary step in a wide variety of clinical and basic research applications. For example, in one approach to identifying lead compounds in drug discovery programs, a large number of compounds are screened for activity as inhibitors or activators of a particular enzyme's activity. As just one example, since abnormal protein phosphorylation has been implicated in a number of diseases and pathological conditions including arthritis, cancer, diabetes, and heart disease, screening for compounds capable of modulating the activity of various protein kinases or protein phosphatases can produce lead compounds for evaluation in treatment of these conditions (see, e.g., Ross et al. (2002) “A non-radioactive method for the assay of many serine/threonine-specific protein kinases” Biochem. J. 366:977-998 and references therein).

Simple and reproducible methods for qualitative and/or quantitative detection of enzyme activity are thus desirable, for drug discovery and a wide variety of other applications. Among other benefits, the present invention provides sensors for detecting enzyme activity, as well as related methods for detection of enzyme activity and for screening for compounds affecting enzyme activity.

### SUMMARY OF THE INVENTION

One aspect of the invention provides a variety of fluorescent sensors for detecting enzyme activity. In general, the enzyme sensors include a substrate for the enzyme and a fluorescent label which is quenched by a quencher until the enzyme acts on the substrate, at which point quenching is relieved and fluorescence increases. Compositions, kits, and systems including the sensors or components thereof and methods for using the sensors to detect enzyme activity and to screen for compounds affecting enzyme activity are also described.

A first general class of embodiments provides a composition including a sensor for detecting an activity of a enzyme. The sensor comprises a substrate for the enzyme and a fluorescent label and a quencher covalently connected to the substrate. The substrate is in a first state on which the enzyme can act, thereby converting the substrate to a second state. When the substrate is in the first state, florescent emission by the label is quenched by the quencher. Conversion of the substrate from the first state to the second state alters the net charge of the substrate, typically introducing an unfavorable intramolecular electrostatic interaction or eliminating a favorable intramolecular electrostatic interaction, and thereby resulting in a conformational change in the sensor that at least partially relieves quenching of the label by the quencher. The intensity of fluorescent emission from the label therefore increases, for example, by at least about 10%.

In one class of embodiments, the substrate is a polypeptide substrate. Optionally, conversion of the substrate from the first state to the second state alters the charge of an amino acid side chain in the polypeptide. In one aspect, conversion of the substrate from the first state to the second state involves transfer of a functional group to the side chain. Examples include, but are not limited to, phosphorylation, acetylation, alkylation (e.g., methylation), glycosylation, and sulfation, involving transfer of a phosphoryl, acetyl, alkyl (e.g., methyl), glycosyl, or sulfyl group to the side chain. Similarly, conversion of the substrate from the first state to the second state can involve removal of a functional group from the side chain, for example, dephosphorylation, demethylation, or deacetylation. The functional group that is added or removed from the side chain can be charged or uncharged. The fluorescent label is optionally positioned adjacent to the residue whose side chain is modified.

In one class of embodiments, the amino acid side chain in the first state is uncharged and in the second state is negatively charged. Exemplary reactions in this class of embodiments include, but are not limited to, sulfation (e.g., of tyrosine side chains) and phosphorylation (e.g., wherein the amino acid side chain is a serine, threonine, or tyrosine side chain which is unphosphorylated in the first state and phosphorylated in the second state). In one exemplary class of embodiments, the quencher is negatively charged, and conversion of the substrate from the first state in which the amino acid side chain is uncharged to the second state in which the side chain is negatively charged introduces an unfavorable electrostatic interaction between the quencher and the side chain. In a related class of embodiments, one or more amino acid residues adjacent to the quencher are negatively charged, and conversion of the substrate from the first state in which the amino acid side chain is uncharged to the second state in which the side chain is negatively charged introduces an unfavorable electrostatic interaction between the side chain and the residues. In one exemplary class of embodiments, the composition includes one of P15-P20. The polypeptide substrate optionally comprises the amino acid sequence of SEQ ID NO:24.

In another class of embodiments, the amino acid side chain in the first state is positively charged and in the second state is uncharged. For example, the amino acid side chain can be an arginine or lysine side chain which is unmethylated in the first state and methylated in the second state, or a lysine side chain which is unacetylated in the first state and acetylated in the second state. In one exemplary class of embodiments, the quencher is negatively charged, and conversion of the substrate from the first state in which the amino acid side chain is positively charged to the second state in which the side chain is uncharged eliminates a favorable electrostatic interaction between the quencher and the side chain. In a related class of embodiments, one or more amino acid residues adjacent to the quencher are negatively charged, and conversion of the substrate from the first state in which the amino acid side chain is positively charged to the second state in which the side chain is uncharged eliminates a favorable electrostatic interaction between the side chain and the residues.

The sensors can be used to detect activity of any of a large number of enzymes, e.g., in in vitro or in-cell assays. Thus, for example, the enzyme is optionally a protein kinase, a serine/threonine protein kinase, a tyrosine protein kinase, a histone methyltransferase, a histone lysine methyltransferase, a histone arginine methyltransferase, a protein lysine methyltransferase, a histone acetyltransferase, a lysine acetyltransferase, or a protein phosphatase.

The composition optionally includes a detection module which binds to the substrate when the substrate is in the second state. Use of such a detection module can assist in relief of quenching by sequestering the label, amplifying the increase in intensity of fluorescent emission from the label. The substrate and the detection module can be part of a single molecule. More typically, however, the substrate comprises a first molecule and the detection module comprises a second molecule. For example, the substrate can comprise a first polypeptide and the detection module a second polypeptide. Exemplary detection modules include, but are not limited to, a 14-3-3 domain, an SH2 domain, a PTB domain, a chromodomain, a bromodomain, and an antibody; other suitable examples are described hereinbelow.

A variety of fluorescent labels are known in the art and can be adapted to the practice of the present invention. In one aspect, the label is pyrene or a coumarin derivative. Similarly, a variety of quenchers are known in the art and can be adapted to the practice of the present invention. Examples include, but are not limited to, Reactive Blue 2, Carminic Acid, Evans Blue, Eriochrome Black T, Alizarin Red, Aniline Blue WS, Chlorazol Black, Ponceau S, Rose Bengal, Tartrazine, Trypan Blue, and Acid Green 27.

In one class of embodiments, the sensor is caged such that the enzyme can not act upon the substrate until the sensor is uncaged. For example, in one embodiment, the sensor comprises one or more photolabile caging groups covalently bound to the substrate, which caging groups inhibit or prevent the enzyme from acting upon the substrate.

The sensors are optionally employed to study the effects of activators and inhibitors (known and potential) on the enzyme's activity. Thus, the composition optionally includes a modulator or potential modulator of the activity of the enzyme.

The composition optionally includes the enzyme, a cell lysate, and/or a cell (e.g., a cell that includes the sensor, the enzyme, a detection module, and/or nucleic acid(s) encoding such detection module or enzyme).

Another general class of embodiments provides a composition that includes a labeled polypeptide, which labeled polypeptide comprises a fluorescent label, a polypeptide, and a quencher that is covalently connected to the polypeptide. The polypeptide comprises amino acid sequence X⁻⁴R⁻³R⁻²X⁻¹S⁰X⁺¹X⁺², where X⁻⁴ and X⁺² are independently selected from the group consisting of an amino acid residue, an amino acid residue comprising the fluorescent label, and an amino acid residue comprising the quencher, and where X⁻¹ and X⁺¹ are independently selected from the group consisting of a hydrophobic amino acid residue, an amino acid residue comprising the fluorescent label, and an amino acid residue comprising the quencher. S⁰ is optionally unphosphorylated or phosphorylated.

The labeled polypeptide can be essentially any of those described herein. Thus, in one class of embodiments, the polypeptide comprises an amino acid sequence selected from the group consisting of SEQ ID NOs:23-24. For example, the labeled polypeptide can be any of P15-P20.

Essentially all of the features noted for the embodiments above apply to these embodiments as well, as relevant: for example, with respect to type of fluorescent label, type of quencher, configuration of the labeled polypeptide, and/or the like. The composition optionally includes a 14-3-3 or similar domain that binds the serine-phosphorylated labeled polypeptide and/or a nucleic acid encoding such a domain, a kinase or phosphatase for which the polypeptide is a substrate and/or a nucleic acid encoding such an enzyme, a cell lysate, and/or a cell (e.g., a cell that includes the labeled polypeptide, a 14-3-3 or similar domain, a kinase or phosphatase, and/or nucleic acid(s) encoding such domain or enzyme).

Another general class of embodiments provides a composition that includes a sensor for detecting an activity of a protein kinase. The sensor comprises a substrate module and a detection module. The substrate module includes a polypeptide substrate for the kinase, wherein the substrate is in a first, unphosphorylated state on which the kinase can act, thereby converting the substrate to a second, phosphorylated state, a fluorescent label, and a quencher. The quencher and typically the label are covalently connected to the substrate. The detection module binds to the substrate module when the substrate is in the second, phosphorylated state. Binding of the detection module to the substrate module results in an increase in intensity of fluorescent emission from the label of at least about 1.5 fold. The composition optionally includes the kinase.

The substrate and detection modules can be part of a single molecule. More typically, however, the substrate module comprises a first molecule and the detection module comprises a second molecule. For example, the substrate module can comprise a first polypeptide and the detection module a second polypeptide.

In one class of embodiments, the protein kinase is a serine/threonine protein kinase. The detection module is optionally, e.g., a polypeptide, an aptamer, or the like that recognizes the phosphorylated serine and/or threonine substrate. For example, the detection module can include a 14-3-3, FHA, WD40, WW, Vhs, HprK, DSP, KIX, MH2, PKI, API3, ARM, cyclin, CDI, or GlgA domain, or an antibody. The substrate and detection modules optionally comprise distinct polypeptides.

In one exemplary class of embodiments, the substrate module comprises a polypeptide substrate comprising amino acid sequence X⁻⁴R⁻³R⁻²X⁻¹S⁰X⁺¹X⁺²; where X⁻⁴ and X⁺² are independently selected from the group consisting of an amino acid residue, an amino acid residue comprising the quencher, and an amino acid residue comprising the fluorescent label; and where X⁻¹ and X⁺¹ are independently selected from the group consisting of a hydrophobic amino acid residue, an amino acid residue comprising the quencher, and an amino acid residue comprising the fluorescent label. The polypeptide substrate optionally comprises the amino acid sequence of SEQ ID NO:23. For example, the substrate module can be AcGAla(Pyr)TGRRDap(Reactive Blue 2)SLPA-amide (P13, SEQ ID NO:21) or AcGAla(Pyr)TGRRDap(Carminic acid)SLPA-amide (P14, SEQ ID NO:22). In one embodiment, the detection module is a 14-3-3 domain.

In another class of embodiments, the protein kinase is a tyrosine protein kinase. The detection module is optionally, e.g., a polypeptide, an aptamer, or the like that recognizes the phosphorylated tyrosine substrate. For example, the detection module can include an SH2 domain, an FHA domain, a PTB domain, or an antibody. The substrate and detection modules optionally comprise distinct polypeptides.

Essentially all of the features noted for the embodiments above also apply to these embodiments as well, as relevant: for example, with respect to configuration of the substrate module, attachment of caging groups to the substrate, inclusion of a modulator or potential modulator of the activity of the kinase, and/or the like. The label is optionally pyrene, and/or the quencher is optionally Reactive Blue 2 or Carminic Acid. The composition optionally includes the kinase (e.g., a purified or partially purified kinase), a cell or tissue lysate (e.g., one including the kinase), or a cell (e.g., a cell comprising the sensor and/or the kinase, a nucleic acid encoding the detection module, and/or a nucleic acid encoding the kinase).

As noted above, methods for using the sensors described herein to detect enzyme activity and to screen for compounds affecting enzyme activity are also a feature of the invention. Accordingly, one general class of embodiments provides methods of assaying an activity of an enzyme. In the methods, the enzyme is contacted with a sensor. The sensor includes a substrate for the enzyme and a fluorescent label and a quencher covalently connected to the substrate. The substrate is in a first state on which the enzyme can act, thereby converting the substrate to a second state. When the substrate is in the first state, florescent emission by the label is quenched by the quencher. Conversion of the substrate from the first state to the second state alters the net charge of the substrate, typically introducing an unfavorable intramolecular electrostatic interaction or eliminating a favorable intramolecular electrostatic interaction, and thereby resulting in a conformational change in the sensor that at least partially relieves quenching of the label by the quencher and results in an increased intensity of fluorescent emission from the label, e.g., of at least about 10%. The increased intensity of fluorescent emission from the label is detected and correlated to the activity of the enzyme, thereby assaying the activity of the enzyme. The assay is optionally qualitative or quantitative.

In one class of embodiments, the sensor comprises one or more caging groups associated with the substrate, which caging groups inhibit (e.g., prevent) the enzyme from acting upon the substrate. The methods include uncaging the substrate, e.g., by exposing the substrate to uncaging energy, thereby freeing the substrate from inhibition by the one or more caging groups. The substrate can be uncaged, for example, by exposing the substrate to light of a first wavelength.

The methods can be used to screen for compounds that affect activity of the enzyme. Thus, in one class of embodiments, the methods include contacting the enzyme with a test compound, assaying the activity of the enzyme in the presence of the test compound, and comparing the activity of the enzyme in the presence of the test compound with the activity of the enzyme in the absence of the test compound.

Essentially all of the features noted for the compositions above apply to these methods as well, as relevant: for example, with respect to type of enzyme and/or substrate, configuration of the sensor, exemplary sensors, type of fluorescent label and/or quencher, contacting the enzyme with a modulator, and/or the like. The methods optionally include contacting the substrate with a detection module that binds to the substrate when the substrate is in the second state. Exemplary detection modules have been described above. The methods optionally include introducing the sensor into a cell.

Another general class of embodiments provides methods of assaying activity of a protein kinase. In the methods, the kinase is contacted with a sensor. The sensor includes a substrate module and a detection module. The substrate module includes a polypeptide substrate for the kinase, wherein the substrate is in a first, unphosphorylated state on which the kinase can act, thereby converting the substrate to a second, phosphorylated state, a fluorescent label, and a quencher. The quencher and typically the label are covalently connected to the substrate. The detection module binds to the substrate module when the substrate is in the second, phosphorylated state. Binding of the detection module to the substrate module results in an increase in intensity of fluorescent emission from the label, preferably, an increase of at least about 1.5 fold. The increase in intensity of fluorescent emission from the label is detected and correlated to the activity of the kinase, thereby assaying the activity of the kinase. As for the embodiments above, the assay is optionally qualitative or quantitative.

Essentially all of the features noted for the compositions and methods above apply to these methods as well, as relevant: for example, with respect to type of kinase, exemplary substrate and/or detection modules, type of fluorescent label and/or quencher, caging and uncaging of the sensor, contacting the kinase with a modulator or test compound, and/or the like. For in-cell assays of kinase activity, for example, the methods optionally include introducing the substrate module and/or detection module into a cell.

### BRIEF DESCRIPTION OF THE DRAWINGS

FIG. 1 Panels A-D schematically illustrate operation of an exemplary serine kinase sensor.

FIG. 2 Panel A depicts the structure of a Dap residue. Panel B depicts the structures of lead quencher dyes of pyrene-peptide fluorescence for pyrene peptides P1-P11.

FIG. 3 presents a graph showing fluorescence fold-change as a function of time in the presence of Rose Bengal/P5 (curve a), Aniline Blue WS/P9 (curve b), or Ponceau S/P2 (curve c) pairs.

FIG. 4 presents graphs showing percent fluorescent quenching of peptide P2 as a function of concentration of the ten lead quenchers.

FIG. 5 Panel A presents a graph showing percent fluorescent quenching of pyrene fluorescence in peptide P5 with Rose Bengal dye (curve a) and peptide P9 with Aniline Blue WS dye (curve b). Panel B presents a graph showing percent fluorescent quenching of pyrene fluorescence in phosphorylated peptide P5 with Rose Bengal.

FIG. 6 presents a graph showing fluorescence as a function of the concentration of peptide P5 before (solid line) and after (dotted line) background correction.

FIG. 7 presents a graph showing PKA-induced fluorescence change of the Rose Bengal/peptide P5 pair in the presence (curve a) and absence (curve b) of 14-3-3τ.

FIG. 8 presents graphs showing fractional PKA activity versus log [inhibitor] for H9-HCl at 10 μM ATP in Panel A, H9.HCl at 1 mM ATP in Panel B, and PKI (14-22) in Panel C, using the deep quench method described herein, and in Panel D PKI (14-22) using the standard radioactive ATP method.

FIG. 9 Panel A depicts the structures of peptide P12 and of Acid Green 27. Panel B presents a graph showing fluorescence fold-change as a function of time for peptide P12 with Acid Green 27.

FIG. 10 Panels A-D schematically illustrate operation of an exemplary tyrosine kinase sensor.

FIG. 11 Panels A-D schematically illustrate operation of an exemplary methyltransferase sensor.

FIG. 12 Panels A-D schematically illustrate operation of an exemplary acetyltransferase sensor.

FIG. 13 Panels A-C schematically illustrate operation of an exemplary binding sensor.

FIG. 14 Panels A-C schematically illustrate operation of an exemplary binding sensor for detection of interaction between a proline-rich peptide and an SH3 domain.

FIG. 15 Panel A depicts the structure of peptide P13. Panel B depicts the structure of peptide P14.

FIG. 16 Panels A-C schematically illustrate operation of an exemplary sensor for the serine kinase PKA.

FIG. 17 Panels A-F present graphs showing PKA-induced fluorescence change of peptides P15-P20 (P15 in Panel A, P16 in Panel B, P17 in Panel C, P18 in Panel D, P19 in Panel E, and P20 in Panel F).

FIG. 18 Panels A-C schematically illustrate operation of an exemplary methyltransferase sensor.

Schematic figures are not necessarily to scale.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an,” and “the” include plural referents unless the context clearly dictates otherwise. Thus, for example, reference to “a molecule” includes a plurality of molecules, and the like.

The term “about” as used herein indicates the value of a given quantity varies by +/−10% of the value, or optionally +/−5% of the value, or in some embodiments, by +/−1% of the value so described.

An “acetyltransferase” is an enzyme that catalyzes the transfer of an acetyl group from one molecule to another. A “lysine acetyltransferase” transfers an acetyl group, typically from acetyl coenzyme A, to the ε-amino group of a lysine residue in a protein. A “histone acetyltransferase” transfers an acetyl group to a histone, e.g., to the ε-amino group of a lysine residue in the histone.

An “amino acid sequence” is a polymer of amino acid residues (a protein, polypeptide, etc.) or a character string representing an amino acid polymer, depending on context.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include multiple or single chain antibodies, including single chain Fv (sFv or scFv) antibodies in which a variable heavy and a variable light chain are joined together (directly or through a peptide linker) to form a continuous polypeptide.

An “aptamer” is a nucleic acid capable of interacting with a ligand. An aptamer can be, e.g., a DNA or RNA, and can be e.g. a chemically synthesized oligonucleotide. The ligand can be any natural or synthetic molecule, including, e.g., the first or second state of a substrate.

A “caging group” is a moiety that can be employed to reversibly block, inhibit, or interfere with the activity (e.g., the biological activity) of a molecule (e.g., a polypeptide, a nucleic acid, a small molecule, a drug, etc.). The caging groups can, e.g., physically trap an active molecule inside a framework formed by the caging groups. Typically, however, one or more caging groups are associated (covalently or noncovalently) with the molecule but do not necessarily surround the molecule in a physical cage. For example, a single caging group covalently attached to an amino acid side chain required for the catalytic activity of an enzyme can block the activity of the enzyme. The enzyme would thus be caged even though not physically surrounded by the caging group. As another example, covalent attachment of a single caging group to an amino acid side chain that is phosphorylated by a kinase in a kinase substrate can block phosphorylation of that substrate by the kinase. Caging groups can be, e.g., relatively small moieties such as carboxyl nitrobenzyl, 2-nitrobenzyl, nitroindoline, hydroxyphenacyl, DMNPE, or the like, or they can be, e.g., large bulky moieties such as a protein or a bead. Caging groups can be removed from a molecule, or their interference with the molecule's activity can be otherwise reversed or reduced, by exposure to an appropriate type of uncaging energy and/or exposure to an uncaging chemical, enzyme, or the like.

A “photoactivatable” or “photoactivated” caging group is a caging group whose blockage of, inhibition of, or interference with the activity of a molecule with which the photoactivatable caging group is associated can be reversed or reduced by exposure to light of an appropriate wavelength. For example, exposure to light can disrupt a network of caging groups physically surrounding the molecule, reverse a noncovalent association with the molecule, trigger a conformational change that renders the molecule active even though still associated with the caging group, or cleave a photolabile covalent attachment to the molecule, etc.

A “photolabile” caging group is one whose covalent attachment to a molecule is reversed (cleaved) by exposure to light of an appropriate wavelength. The photolabile caging group can be, e.g., a relatively small moiety such as carboxyl nitrobenzyl, 2-nitrobenzyl, nitroindoline, hydroxyphenacyl, DMNPE, or the like, or it can be, e.g., a relatively bulky group (e.g. a macromolecule, a protein) covalently attached to the molecule by a photolabile linker (e.g., a polypeptide linker comprising a 2-nitrophenyl glycine residue).

A “Dab residue” is an (L)-2,4-diaminobutyric acid residue.

A “Dap residue” is an (L)-2,3-diaminopropionic acid residue.

An “enzyme” is a biological macromolecule that has at least one catalytic activity (i.e., that catalyzes at least one chemical reaction). An enzyme is typically a protein, but can be, e.g., RNA. Known protein enzymes have been grouped into six classes (and a number of subclasses and sub-subclasses) under the Enzyme Commission classification scheme (see, e.g. the Nomenclature Committee of the International Union of Biochemistry and Molecular Biology enzyme nomenclature pages, on the world wide web at www (dot) chem (dot) qmul (dot) ac (dot) uk/iubmb/enzyme), namely, oxidoreductase, transferase, hydrolase, lyase, ligase, or isomerase. The activity of an enzyme can be “assayed,” either qualitatively (e.g., to determine if the activity is present) or quantitatively (e.g., to determine how much activity is present or kinetic and/or thermodynamic constants of the reaction).

A “kinase” is an enzyme that catalyzes the transfer of a phosphate (phosphoryl) group from one molecule to another. A “protein kinase” is a kinase that transfers a phosphate group to a protein, typically from a nucleotide such as ATP. A “tyrosine protein kinase” (or “tyrosine kinase”) transfers the phosphate to a tyrosine side chain (e.g., a particular tyrosine), while a “serine/threonine protein kinase” (“serine/threonine kinase”) transfers the phosphate to a serine or threonine side chain (e.g., a particular serine or threonine).

A “label” is a moiety that facilitates detection of a molecule. Fluorescent labels are preferred labels in the context of the invention. Many labels are known in the art and commercially available and can be used in the context of the invention.

An “environmentally sensitive label” is a label whose signal changes when the environment of the label changes. For example, the fluorescence of an environmentally sensitive fluorescent label changes when the hydrophobicity, pH, and/or the like of the label's environment changes (e.g., upon binding of the molecule with which the label is associated to another molecule such that the label is transferred from an aqueous environment to a more hydrophobic environment at the molecular interface).

A “methyltransferase” is an enzyme that catalyzes the transfer of a methyl group from one molecule to another. A “protein lysine methyltransferase” transfers a methyl group to the ε-amino group of a lysine residue in a protein. A “histone methyltransferase” transfers a methyl group, e.g., from S-adenosyl methionine, to a histone; a “histone lysine methyltransferase” transfers a methyl group to a lysine residue in a histone, while a “histone arginine methyltransferase” transfers a methyl group to an arginine residue in a histone.

A “modulator” enhances or inhibits an activity of an enzyme or protein (e.g., a catalytic activity of an enzyme), either partially or completely. An “activator” enhances the activity (whether moderately or strongly). An “inhibitor” inhibits the activity (e.g., an inhibitor of an enzyme attenuates the rate and/or efficiency of catalysis), whether moderately or strongly. A modulator can be, e.g., a small molecule, a polypeptide, a nucleic acid, etc.

The term “nucleic acid” encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA in solution, such as 2′-O-methylated oligonucleotides), and the like. The nucleotides of the nucleic acid can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The nucleic acid can additionally comprise non-nucleotide elements such as labels, quenchers, blocking groups, or the like. A nucleic acid can be e.g., single-stranded or double-stranded. Unless otherwise indicated, a particular nucleic acid sequence of this invention encompasses complementary sequences, in addition to the sequence explicitly indicated.

A “phosphatase” is an enzyme that removes a phosphate group from a molecule. A “protein phosphatase” removes the phosphate group from an amino acid side chain in a protein. A “serine/threonine-specific protein phosphatase” removes the phosphate from a serine or threonine side chain (e.g., a particular serine or threonine), while a “tyrosine-specific protein phosphatase” removes the phosphate from a tyrosine side chain (e.g., a particular tyrosine).

A “polypeptide” is a polymer comprising two or more amino acid residues (e.g., a peptide or a protein). The polymer can additionally comprise non-amino acid elements such as labels, blocking groups, or the like and can optionally comprise modifications such as glycosylation or the like. The amino acid residues of the polypeptide can be natural or non-natural and can be unsubstituted, unmodified, substituted or modified.

A “quencher” is a moiety that alters a property of a label (typically, a fluorescent label) when it is in proximity to the label. For example, the quencher can quench (reduce the intensity of) a fluorescent emission (e.g., at a particular wavelength) from a fluorescent label when it is proximal to the label as compared to when not proximal to the label. A quencher can be, e.g., an acceptor fluorophore that operates via energy transfer and re-emits the transferred energy as light. Other similar quenchers, called “dark quenchers,” do not re-emit transferred energy via fluorescence.

A “substrate” is a molecule on which an enzyme acts. The substrate is typically supplied in a first state on which the enzyme acts, converting it to a second state. The second state of the substrate (product) is then typically released from the enzyme.

“Uncaging energy” is energy that removes one or more caging groups from a caged molecule (or otherwise reverses the caging groups' blockage of the molecule's activity). As appropriate for the particular caging group(s), uncaging energy can be supplied, e.g., by light, sonication, a heat source, a magnetic field, or the like.

A variety of additional terms are defined or otherwise characterized herein.

### DETAILED DESCRIPTION

In one aspect, the invention provides a variety of fluorescent sensors for detecting enzyme activity. In general, the sensors include a substrate for the enzyme and a fluorescent label which is quenched by a quencher until the enzyme acts on the substrate, at which point quenching is relieved and fluorescence increases. In one class of embodiments, the sensor includes a substrate module, a quencher, and a detection module. The substrate module includes a substrate for the enzyme of interest and a fluorescent label. The detection module binds to the substrate module either before or after the enzyme acts on the substrate and sequesters the label from the quencher, resulting in an increased signal from the label. In certain embodiments, the quencher is not covalently bound to the substrate or the detection module, while in other embodiments, the quencher is covalently bound to the substrate. In another class of embodiments, the sensor includes a substrate module comprising a substrate for the enzyme of interest, a fluorescent label, and a covalently bound quencher. Action of the enzyme on the substrate leads to a conformational change in the sensor and relief of quenching. In these embodiments, use of a detection module is optional. Compositions, kits, and systems including the sensors or components thereof and methods for using the sensors to detect enzyme activity and to screen for compounds affecting enzyme activity are described.

In another aspect, the invention provides a variety of sensors for detecting protein-protein interactions. In one class of embodiments, the binding sensor includes a quencher and a labeled polypeptide that comprises a first polypeptide and a label. Binding of the first polypeptide to a second polypeptide sequesters the label from the quencher, resulting in an increased signal from the label. Compositions, kits, and systems including the binding sensors or components thereof and methods for using the sensors to detect protein-protein interactions and to screen for compounds affecting protein-protein interactions are described.

### Enzyme Sensors

A first general class of embodiments provides a composition including a sensor for detecting an activity of an enzyme. The sensor comprises a substrate module, a detection module, and a quencher. The substrate module includes a substrate for the enzyme, wherein the substrate is in a first state on which the enzyme can act, thereby converting the substrate to a second state, and a fluorescent label. The detection module binds to the substrate module when the substrate is in the first state or when the substrate is in the second state. Binding of the detection module to the substrate module results in an increased intensity of fluorescent emission from the label, since the label is at least partially sequestered from the quencher. In one aspect, the quencher is not covalently bound to the substrate module or to the detection module. The composition optionally includes the enzyme.

The substrate and detection modules can be part of a single molecule. More typically, however, the substrate module comprises a first molecule and the detection module comprises a second molecule. For example, the substrate module can comprise a first polypeptide and the detection module a second polypeptide. It is worth noting that the substrate module can comprise essentially any suitable substrate, for example, one or more of an amino acid, a polypeptide, a nitrogenous base, a nucleoside, a nucleotide, a nucleic acid, a carbohydrate, a lipid, or the like. The substrate is optionally a specific substrate (acted on only by a single type of catalytic molecule, e.g., under a defined set of reaction conditions), or a generic substrate (acted on by more than one member of a class of catalytic molecules). Similarly, the detection module can comprise essentially any molecule that can bind the first or second state of the substrate, for example, a polypeptide, an aptamer, or the like.

The enzyme whose activity is to be detected can be essentially any enzyme. For example, the enzyme can be a transferase, or it can be an oxidoreductase, hydrolase, lyase, ligase, or isomerase. In one embodiment, the enzyme catalyzes a posttranslational modification of a polypeptide, for example, phosphorylation, acetylation, methylation, ubiquitination, sumoylation, glycosylation, prenylation, myristoylation, farnesylation, attachment of a fatty acid, attachment of a GPI anchor, nucleotidylation (e.g., ADP-ribosylation), or the like. For example, the enzyme can be a transferase from any one of EC subclasses 2.1-2.9 (e.g., a glycosyltransferase, protein farnesyltransferase, or protein geranylgeranyltransferase), a ligase from any one of EC subclasses 6.1-6.6 (e.g., a ubiquitin transferase or ubiquitin-conjugating enzyme), or a hydrolase from any one of EC subclasses 3.1-3.13 (e.g., a phosphatase or glycosylase). The enzyme is optionally an enzyme that does not cleave its substrate (that is, optionally conversion of the substrate from the first state to the second state does not involve cleavage of the substrate by the enzyme).

In one preferred class of embodiments, the enzyme is a protein kinase. The substrate is therefore a substrate for a protein kinase, e.g., a polypeptide substrate for the kinase. In this class of embodiments, the substrate in the first state is unphosphorylated (not phosphorylated), and the substrate in the second state is phosphorylated. In some embodiments, the detection module binds to the substrate module when the substrate is in the first state; in other embodiments, the detection module binds to the substrate module when the substrate is in the second state (i.e., the detection module binds to the phosphorylated substrate). It is worth noting that, in this class of embodiments as well as other embodiments herein, while the detection module can bind to the substrate in either the first state or the second state, embodiments in which the detection module binds to the substrate in the second state are generally preferable since in these embodiments the detection module is not competing with the enzyme for the substrate.

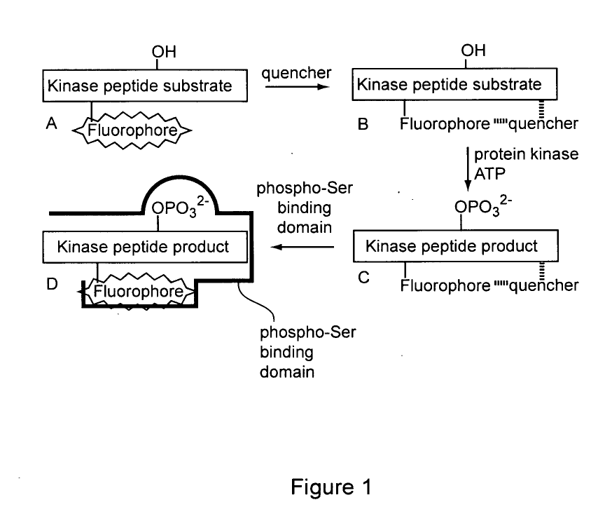

In one class of embodiments, the protein kinase is a serine/threonine protein kinase. The detection module is optionally, e.g., a polypeptide, an aptamer, or the like that recognizes the phosphorylated serine and/or threonine substrate. For example, the detection module can include a 14-3-3, FHA, WD40, WW, Vhs, HprK, DSP, KIX, MH2, PKI, API3, ARM, cyclin, CDI, or GlgA domain, or an antibody. The substrate and detection modules optionally comprise distinct polypeptides. See, for example, the embodiment schematically illustrated in FIG. 1.

In one exemplary class of embodiments, the substrate module comprises a polypeptide substrate comprising amino acid sequence X⁻⁴R⁻³R⁻²X⁻¹S⁰X⁺¹X⁺²; where X⁻⁴ and X⁺² are independently selected from the group consisting of an amino acid residue and an amino acid residue comprising the fluorescent label; and where X⁻¹ and X⁺¹ are independently selected from the group consisting of a hydrophobic amino acid residue (e.g., Phe, Leu, Ile, etc.) and an amino acid residue comprising the fluorescent label. The label is optionally attached to one of X⁻⁴, X⁻¹, X⁺¹ and X⁺², or to a residue or other moiety N-terminal of X⁻⁴ or C-terminal of X⁺². The composition optionally includes a cAMP-dependent protein kinase (PKA) that can phosphorylate S⁰. The termini of the polypeptide are optionally free or modified; for example, the N-terminus can be free or acetylated and/or the C-terminus can be a free carboxyl or a C-terminal amide. The polypeptide substrate optionally comprises an amino acid sequence selected from the group consisting of SEQ ID NOs:13-18.

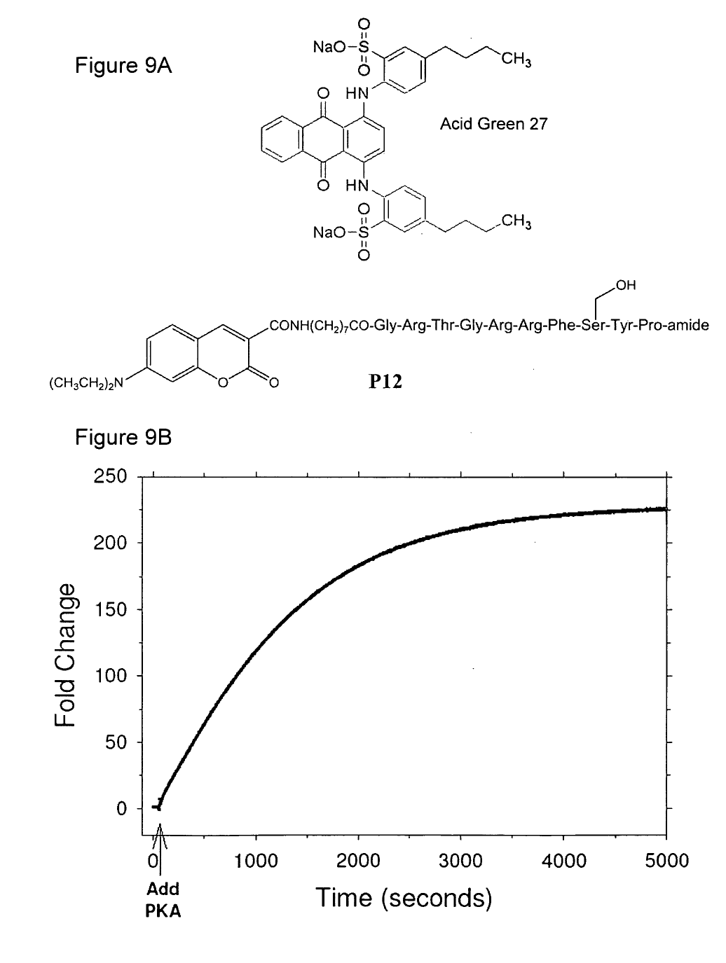

For example, the substrate module can be any one of P1-P12 (which are described in the Examples sections herein below; see, e.g., Table 1 and FIG. 9 Panel A), or it can comprise the amino acid sequence of any one of P1-P12 and have a label (e.g., pyrene or a coumarin derivative) attached to the corresponding residue. As noted, the termini of the polypeptide are optionally free or modified. In a few specific examples, the detection module is a 14-3-3 domain, and the substrate module is P5 and the quencher is Rose Bengal, the substrate module is P9 and the quencher is Aniline Blue WS, the substrate module is P2 and the quencher is Ponceau S, or the substrate module is P12 and the quencher is Acid Green 27. A number of additional exemplary sensors are described in the Examples section below.

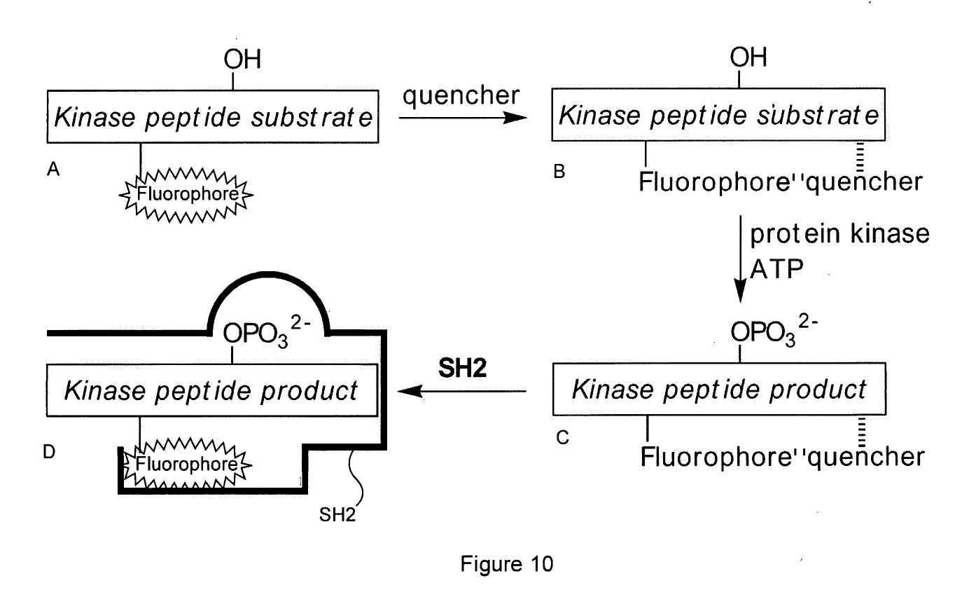

In another class of embodiments, the protein kinase is a tyrosine protein kinase. The detection module is optionally, e.g., a polypeptide, an aptamer, or the like that recognizes the phosphorylated tyrosine substrate. For example, the detection module can include an SH2 domain, an FHA domain, a PTB (phosphotyrosine binding) domain, or an antibody. The substrate and detection modules optionally comprise distinct polypeptides. See, for example, the embodiment schematically illustrated in FIG. 10.

Substrate and/or detection modules for use in the tyrosine protein kinase sensors are optionally adapted from those described in U.S. patent application Ser. No. 11/366,221 filed Mar. 1, 2006 entitled “Enzyme sensors including environmentally sensitive or fluorescent labels and uses thereof” by David S. Lawrence et al. Thus, in one exemplary class of embodiments, the fluorescent label is an environmentally sensitive fluorescent label; the substrate module includes a polypeptide comprising amino acid sequence X⁻⁴X⁻³X⁻²X⁻¹Y⁰X⁺¹X⁺²X⁺³X⁺⁴X⁺⁵; where X⁻⁴, X⁻³, and X⁻² are independently selected from the group consisting of D, E, and an amino acid residue comprising the environmentally sensitive label; X⁻¹ and X⁺³ are independently selected from the group consisting of: A, V, I, L, M, F, Y, W, and an amino acid residue comprising the environmentally sensitive label; X⁺¹, X⁺², X⁺⁴, and X⁺⁵ are independently selected from the group consisting of: an amino acid residue (e.g., a naturally occurring amino acid residue) and an amino acid residue comprising the environmentally sensitive label; and at least one of X⁻⁴, X⁻³, X⁻², X⁻¹, X⁺¹, X⁺², X⁺³, X⁺⁴, and X⁺⁵ is an amino acid residue comprising the environmentally sensitive label; and the detection module optionally comprises an SH2 domain. In other embodiments, the protein kinase can be, e.g., a histidine kinase, an asp/glu kinase, or an arginine kinase.

The phosphopeptide binding domains noted above, as well as other phosphopeptide binding domains, have been well described in the literature. For example, the specificity of various SH2 domains for sequences surrounding the phosphorylated tyrosine residue has been determined. See, e.g., a list of phosphopeptide binding domains at folding (dot) cchmc (dot) org/online/SEPdomaindatabase (dot) htm; a list of protein interaction domains at www (dot) mshri (dot) on (dot) ca/pawson/domains (dot) html; a list of protein domains at www (dot) cellsignal (dot) com/reference/domain/index (dot) asp, which includes consensus binding sites, exemplary peptide ligands, and exemplary binding partners, e.g., for SH-2, 14-3-3, PTB, and WW domains; Kuriyan and Cowburn (1997) “Modular peptide recognition domains in eukaryotic signaling” Annu. Rev. Biophys. Biomol. Struct. 26:259-288; Sharma et al. (2002) “Protein-protein interactions: Lessons learned” Curr. Med. Chem.—Anti-Cancer Agents 2:311-330; Pawson et al. (2001) “SH2 domains, interaction modules and cellular wiring” Trends Cell Biol. 11:504-11; Forman-Kay and Pawson (1999) “Diversity in protein recognition by PTB domains” Curr Opin Struct Biol. 9:690-5; and Fu et al. (2000) “14-3-3 Proteins: Structure, Function, and Regulation” Annual Review of Pharmacology and Toxicology 40:617-647. A large number of such domains from a variety of different proteins have been described, and others can readily be identified, e.g., through sequence alignment, structural comparison, and similar techniques, as is well known in the art. Common sequence repositories for known proteins include GenBank and Swiss-Prot, and other repositories can easily be identified by searching the internet. Similarly, antibodies against phosphotyrosine, phosphoserine, and/or phosphothreonine are well known in the art; many are commercially available, and others can be generated by established techniques. Other domains suitable for use as detection modules include, e.g., death domains, PDZ domains, and SH3 domains. The detection module is optionally a polypeptide (e.g., a recombinant polypeptide, e.g., based on fibronectin) selected for binding to the first or second state of the substrate by a technique such as phage display, mRNA display, or another in vitro or in vivo display and/or selection technique.

A large number of kinases and kinase substrates have been described in the art and can be adapted to the practice of the present invention. For example, the enzyme can be chosen from any of sub-subclasses EC 2.7.10-2.7.12. In one class of embodiments, the kinase is a soluble (non-receptor) tyrosine kinase (for example, Abl, Arg, Blk, Bmx, Brk, BTK, Crk, Csk, DYRK1A, FAK, Fer, Fes/Fps, Fgr, Fyn, Hck, Itk, JAK, Lck, Lyn, MINK, Pyk, Src, Syk, Tec, Tyk, Yes, or ZAP-70), a receptor tyrosine kinase (for example, KIT, MET, KDR, EGFR, or an Eph receptor tyrosine kinase such as EphA1, EphA2, EphA3, EphA4, EphA5, EphA7, EphB1, EphB3, EphB4, or EphB6), a member of a MAP kinase pathway (for example, ARAF1, BRAF1, GRB2, MAPK1, MAP2K1, RASA1, SOS1, MAP2K2, and MAPK3; see, e.g., Cobb et al. (1996) Promega Notes Magazine 59:37-41), a member of an Akt signal pathway (e.g., PTEN, CDKN1A, GSK3B, PDPK1, CDKN1B, ILK, AKT1, PIK3CA, and CCND1), or a member of an EGFR signal pathway (e.g., EGFR, ARAF1, BRAF1, GRB2, MAPK1, MAP2K1, RASA1, SOS1, and MAP2K2). Exemplary kinases include, but are not limited to, Src; AMP-K, AMP-activated protein kinase; PARK, β adrenergic receptor kinase; CaMK, CaM-kinase, calmodulin-dependent protein kinase; cdc2 kinase, protein kinase expressed by CDC2 gene; cdk, cyclin dependent kinase; CK1, protein kinase CK1 (also termed casein kinase 1 or I); CK2, protein kinase CK2 (also termed casein kinase 2 or H); CSK, C-terminal Src protein kinase; GSK3, glycogen synthase kinase-3; HCR, heme controlled repressor, HR1; HMG-CoA reductase kinase A; insulin receptor kinase; MAP kinase, ERK, extracellular signal-regulated kinase; MAP kinase activated protein kinase 1; MAP kinase activated protein kinase 2; MLCK, myosin light chain kinase; Nek, NIMA-related kinase; NIMA, never in mitosis protein kinase; p70 s6k and p90 srk, 70 and 90 kDa kinases that phosphorylate s6 protein; PDHK, pyruvate dehydrogenase kinase; PhK, phosphorylase kinase; PKA, cAMP-dependent protein kinase A; PKB, protein kinase B; PKG, cGMP-dependent protein kinase, protein kinase G; PKR, RNA-dependent protein kinase, dSRNA-PK; PKC, protein kinase C; PRK1, protein kinase C-related kinase 1; RAC; RhK, rhodopsin kinase; SNF-1 PK, sucrose non-fermenting protein kinase; Jun kinase, JNK; JNKKK; SrcN1, SrcN2, FynT, LynA, LynB, FGFR, TrkA, Flt3, and RSK.

Substrates for such kinases, including, e.g., protein substrates (e.g., another kinase, a histone, or myelin basic protein), amino acid polymers of random sequence (e.g., poly Glu/Tyr {4:1}), and/or polypeptide substrates with a defined amino acid sequence (e.g., chemically synthesized polypeptides; polypeptides including less than about 32 residues, less than about 20 residues, or less than about 15 residues; and polypeptides including between 7 and 15 residues), have been described in the art or can readily be determined by techniques known in art. See, e.g., Pinna and Ruzzene (1996) “How do protein kinases recognize their substrates?” Biochim Biophys Acta 1314:191-225. See, e.g., U.S. patent application Ser. No. 11/366,221 for a list of exemplary kinases and polypeptide substrates.

In another class of embodiments, the enzyme is a protein phosphatase. In this class of embodiments, the substrate in the first state is phosphorylated, and the substrate in the second state is unphosphorylated. In some embodiments, the detection module binds to the substrate module when the substrate is in the second state; in other embodiments, the detection module binds to the substrate module when the substrate is in the first state (i.e., the detection module binds to the phosphorylated substrate). Exemplary detection modules for the latter embodiments include those outlined above, e.g., SH2, PTB, 14-3-3, and other phosphoprotein binding domains, as well as antibodies and aptamers.

The phosphatase can be, e.g., a tyrosine-specific protein phosphatase (see, e.g., Alonso et al. (2004) “Protein Tyrosine Phosphatases in the Human Genome” Cell 117:699-711) or a serine/threonine-specific protein phosphatase (e.g., PP1, PP2A, PP2B, or PP2C). See also U.S. patent application Ser. No. 11/366,221. It will be evident that a phosphorylated kinase sensor (for example, phosphorylated versions of the exemplary kinase sensors described herein) can serve as a phosphatase sensor (and vice versa).

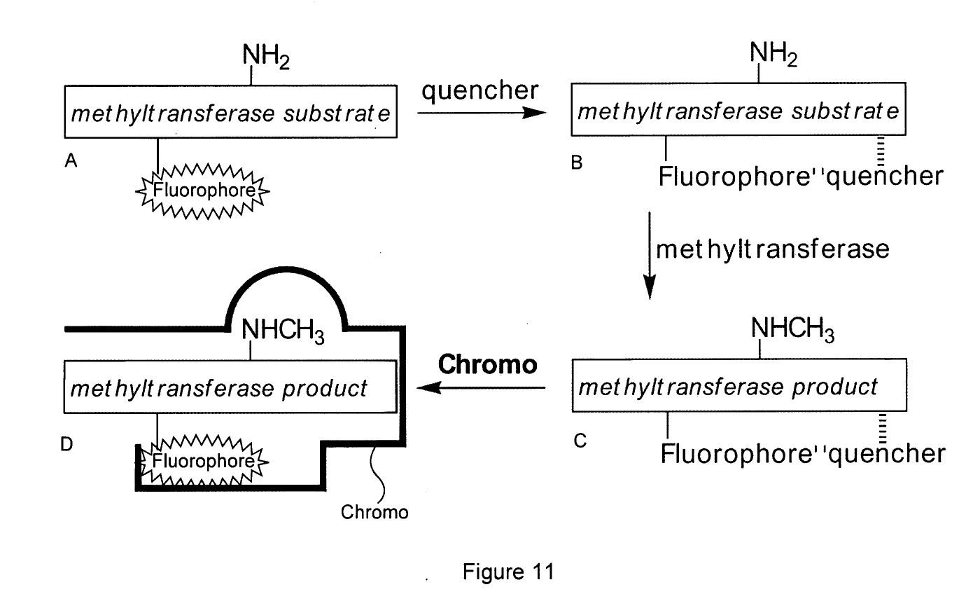

In another class of embodiments, the enzyme is a protein methyltransferase. For example, the enzyme can be a histone methyltransferase (e.g., a histone lysine methyltransferase or a histone arginine methyltransferase) or a protein lysine methyltransferase. In this class of embodiments, the substrate in the first state is unmethylated, and the substrate in the second state is methylated. The detection module is optionally, e.g., a polypeptide, an aptamer, or the like that recognizes the methylated substrate. For example, the detection module can include a chromodomain that binds a substrate including a methyllysine (see, e.g., the embodiment schematically illustrated in FIG. 11), a tudor domain that binds a substrate including a methylarginine, or an antibody. The substrate and detection modules optionally comprise distinct polypeptides.

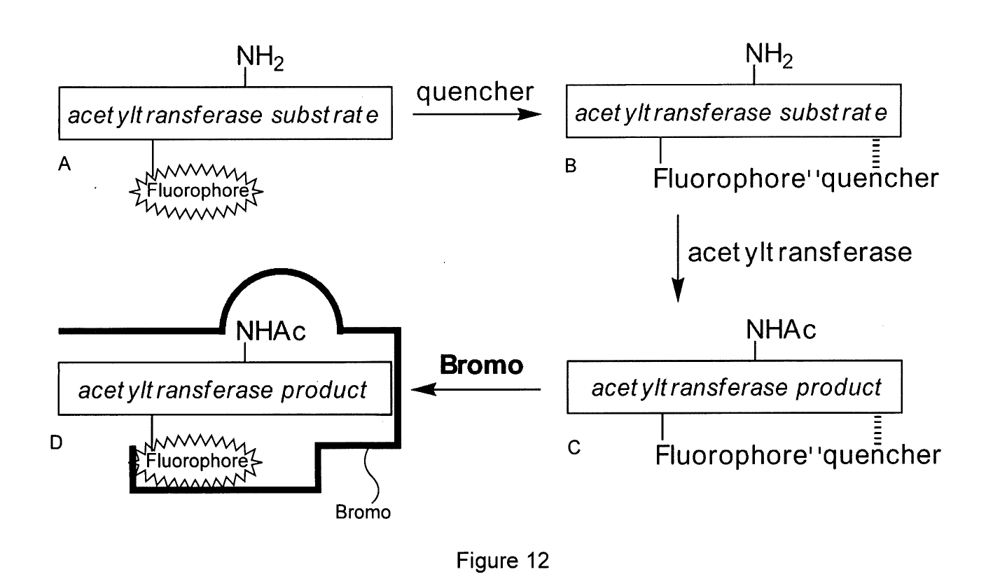

In yet another class of embodiments, the enzyme is a protein acetyltransferase. For example, the enzyme can be a histone acetyltransferase or a lysine acetyltransferase. In this class of embodiments, the substrate in the first state is unacetylated, and the substrate in the second state is acetylated. The detection module is optionally, e.g., a polypeptide, an aptamer, or the like that recognizes the acetylated substrate. For example, the detection module can include a bromodomain that binds a substrate including an acetyllysine, or an antibody; see, e.g., the embodiment schematically illustrated in FIG. 12. The substrate and detection modules optionally comprise distinct polypeptides.

Methyltransferases, acetyltransferases, bromodomains and chromodomains have been described in the art. See, e.g., Yang (2004) “Lysine acetylation and the bromodomain: a new partnership for signaling” BioEssays 26:1076-1087, Berger (2002) “Histone modifications in transcriptional regulation” Curr Opin Genet Dev 12:142-148, Peterson and Laniel (2004) “Histones and histone modifications” Curr Biol 14:R546-R551, and Daniel et al. (2005) “Effector proteins for methylated histones” Cell Cycle 4:919-926.

A variety of fluorescent labels are known in the art and can be adapted to the practice of the present invention. In one aspect, the label is pyrene or a coumarin derivative. Further details can be found in the section entitled “Fluorescent labels” below. The label is generally covalently connected to the substrate.

The increase in signal from the fluorescent label upon binding of the substrate and detection modules can be substantial. For example, the increased intensity of fluorescent emission from the label is optionally an increase of at least about 7 fold, at least about 10 fold, at least about 20 fold, at least about 50 fold, at least about 60 fold, at least about 100 fold, or at least about 200 fold.

The substrate module optionally comprises a polypeptide comprising a Dap, Dab, ornithine, lysine, cysteine, or homocysteine residue (or essentially any other chemically reactive natural or unnatural amino acid derivative or residue) to which the fluorescent label is attached. The label can be attached to the residue (e.g., before or after its incorporation into a polypeptide) by reacting a derivative of the label with a functional group on the residue's side chain, for example. The label can be similarly attached to a free N-terminal amine on the polypeptide by reacting a derivative of the label with the amine, or the label can be introduced by incorporating a phosphoramidite including the label during chemical synthesis of the polypeptide, for example.

A variety of quenchers are known in the art and can be adapted to the practice of the present invention. See, for example, quenchers D1-D48 in Table 2 below. In one class of embodiments, the quencher is selected from the group consisting of Evans Blue, Reactive Blue 2, Eriochrome Black T, Alizarin Red, Aniline Blue WS, Chlorazol Black, Ponceau S, Rose Bengal, Tartrazine, Trypan Blue, and Acid Green 27. The quencher can be, e.g., an acceptor fluorophore, or it can be a dark quencher. In embodiments in which the quencher is a fluorophore, it is preferably a different fluorophore from the fluorescent label. The quencher is typically non-polymeric and is typically a small molecule (e.g., having a molecular weight of less than 1000 daltons, e.g., less than 500 daltons).

Preferably, when the substrate module is not bound to the detection module, the label exhibits little or no fluorescence. Thus, in one aspect, when the substrate module is not bound to the detection module, the quencher quenches fluorescent emission by the label by at least about 40%, as compared to fluorescent emission in the absence of the quencher. For example, the quencher can quench fluorescent emission by the label by at least about 50%, at least about 75%, at least about 90%, or at least about 95%, or can even prevent detectable emission from the label, e.g., at a given wavelength.

The quencher can quench fluorescent emission from the label when the label and quencher are in proximity, e.g., in solution. In one aspect, the quencher forms a non-covalent complex with the substrate module, putting the quencher in proximity to the label. The complex is stabilized by non-covalent interactions between the quencher and the label and/or substrate; for example, by electrostatic interactions, hydrophobic interactions, and/or hydrogen bonds between the quencher and the label and/or substrate (e.g., by electrostatic interactions between a negatively charged moiety on the quencher and positively charged side chain(s) on a polypeptide substrate and/or by hydrophobic interactions between the quencher and the label). Binding of the detection module to the substrate module disrupts the interactions between the quencher and the substrate module, disrupting the complex between the quencher and the substrate module and thereby increasing the intensity of fluorescent emission from the label. In one class of embodiments, the non-covalent complex between the quencher and the substrate module has an apparent dissociation constant (apparent K_{d}) of about 20 μM or less, e.g., about 10 μM or less or even about 1 μM or less.

The molar ratio of the quencher to the substrate module in the composition can be varied, e.g., to achieve a desired level of quenching in the absence of binding of the substrate module to the detection module. For example, the molar ratio of the quencher to the substrate module in the composition can be at least about 1 to 1, at least about 5 to 1, at least about 10 to 1, at least about 25 to 1, or at least about 50 to 1.

The molar ratio of the detection module to the substrate module in the composition is optionally about 1 to 1. Typically, however, the detection module is present in excess (e.g., slight excess) relative to the substrate module. Thus, the molar ratio of the detection module to the substrate module in the composition is optionally greater than 1 to 1; for example, the molar ratio of the detection module to the substrate module can be at least about 2 to 1, at least about 5 to 1, or at least about 10 to 1.

The sensors can be used, e.g., in biochemical assays of enzyme activity. Thus, the composition optionally includes the enzyme (e.g., a purified or partially purified enzyme), a cell or tissue lysate (e.g., a lysate including the enzyme), or a cell.

In one class of embodiments, the sensor is caged such that the enzyme can not act upon the substrate until the sensor is uncaged, for example, by removal of a photolabile caging group. Thus, in one class of embodiments, the sensor comprises one or more caging groups associated with the substrate module (e.g., with the substrate). The caging groups inhibit the enzyme from acting upon the substrate, e.g., by at least about 75%, at least about 90%, at least about 95%, or at least about 98%, as compared to the substrate in the absence of the one or more caging groups. Preferably, the one or more caging groups prevent the enzyme from acting upon the substrate. Typically, removal of, or an induced conformational change in, the one or more caging groups permits the enzyme to act upon the substrate. The one or more caging groups associated with the substrate module can be covalently or non-covalently attached to the substrate module. In a preferred aspect, the one or more caging groups are photoactivatable (e.g., photolabile). For example, in one embodiment, the sensor comprises one or more photolabile caging groups covalently bound to the substrate, which caging groups inhibit or prevent the enzyme from acting upon the substrate. Caging groups are described in greater detail below, in the section entitled “Caging groups”.

Caging of the sensor permits initiation of the reaction between the enzyme and the substrate within the sensor to be controlled, temporally and/or spatially. Similar or additional control of the reaction can be obtained through use of other caged reagents, for example, caged nucleotides (e.g., caged ATP), caged metal ions, caged chelating agents (e.g., caged EDTA or EGTA), caged activators or inhibitors, and the like. See, e.g., US patent application publication 2004/0166553 by Nguyen et al. entitled “Caged sensors, regulators and compounds and uses thereof.”

The sensor can be used to study the effects of activators and inhibitors (known and potential) on the enzyme's activity. Thus, the composition optionally includes a modulator or potential modulator of the activity of the enzyme.

Two or more enzyme activities can be monitored simultaneously or sequentially, if desired, by including in the composition a second sensor. The second sensor can, for example, comprise a second substrate module including a second substrate for a second enzyme and a second fluorescent label, whose signal is detectably different from that of the first sensor's label, and a second detection module. A second quencher is optionally also included, or, preferably, the same type of quencher quenches both labels. The second detection module can be the same as or different from the first detection module.

Quenched Kinase Sensors

As noted above, in one aspect, the quencher is not covalently connected to the substrate or detection module. In another aspect, however, the quencher is covalently bound to the substrate (or the detection module).

For example, one general class of embodiments provides kinase sensors in which the quencher is covalently bound to the kinase substrate. This general class of embodiments provides a composition that includes a sensor for detecting an activity of a protein kinase. The sensor comprises a substrate module and a detection module. The substrate module includes a polypeptide substrate for the kinase, wherein the substrate is in a first, unphosphorylated state on which the kinase can act, thereby converting the substrate to a second, phosphorylated state, a fluorescent label, and a quencher. The quencher and typically the label are covalently connected to the substrate. The detection module binds to the substrate module when the substrate is in the second, phosphorylated state. Binding of the detection module to the substrate module results in an increase in intensity of fluorescent emission from the label, since the label is at least partially sequestered from the quencher. Preferably, the increase in intensity is an increase of at least about 1.5 fold, for example, at least about 2 fold, at least about 2.3 fold, or at least about 5 fold or more. The composition optionally includes the kinase.

The substrate and detection modules can be part of a single molecule. More typically, however, the substrate module comprises a first molecule and the detection module comprises a second molecule. For example, the substrate module can comprise a first polypeptide and the detection module a second polypeptide. The substrate is optionally a specific substrate (acted on only by a single kinase, e.g., under a defined set of reaction conditions), or a generic substrate (acted on by more than one member of a family of kinases). As for the embodiments above, the detection module can comprise essentially any molecule that can bind the second state of the substrate, for example, a polypeptide, an aptamer, or the like.

In one class of embodiments, the protein kinase is a serine/threonine protein kinase. The detection module is optionally, e.g., a polypeptide, an aptamer, or the like that recognizes the phosphorylated serine and/or threonine substrate. For example, the detection module can include a 14-3-3, FHA, WD40, WW, Vhs, HprK, DSP, KIX, MH2, PKI, API3, ARM, cyclin, CDI, or GlgA domain, or an antibody. The substrate and detection modules optionally comprise distinct polypeptides.

In one exemplary class of embodiments, the substrate module comprises a polypeptide substrate comprising amino acid sequence X⁻⁴R⁻³R⁻²X⁻¹S⁰X⁺¹X⁺²; where X⁻⁴ and X⁺² are independently selected from the group consisting of an amino acid residue, an amino acid residue comprising the quencher, and an amino acid residue comprising the fluorescent label; and where X⁻¹ and X⁺¹ are independently selected from the group consisting of a hydrophobic amino acid residue (e.g., Phe, Leu, Ile, etc.), an amino acid residue comprising the quencher, and an amino acid residue comprising the fluorescent label. The label is optionally attached to one of X⁻⁴, X⁻¹, X⁺¹ and X⁺², or to a residue or other moiety N-terminal of X⁻⁴ or C-terminal of X⁺². The composition optionally includes a cAMP-dependent protein kinase (PKA) that can phosphorylate S⁰. The termini of the polypeptide are optionally free or modified; for example, the N-terminus can be free or acetylated and/or the C-terminus can be a free carboxyl or a C-terminal amide. One or more additional amino acid residues are optionally present at the N- and/or C-termini of the specified sequence. The polypeptide substrate optionally comprises the amino acid sequence of SEQ ID NO:23.

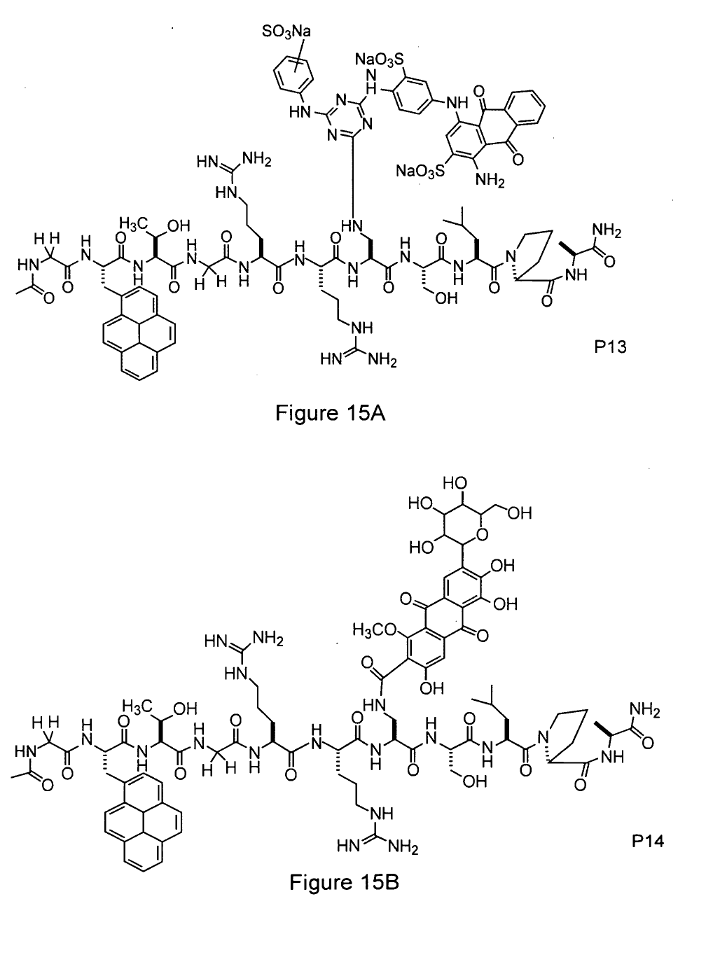

For example, the substrate module can be P13 or P14 (which are described in the Examples sections herein below; see, e.g., FIG. 15 Panels A-B), or it can comprise the amino acid sequence of P13 or P14 and have a label (e.g., pyrene) attached to the corresponding residue. As noted, the termini of the polypeptide are optionally free or modified. In one embodiment, the detection module is a 14-3-3 domain.

In another class of embodiments, the protein kinase is a tyrosine protein kinase. The detection module is optionally, e.g., a polypeptide, an aptamer, or the like that recognizes the phosphorylated tyrosine substrate. For example, the detection module can include an SH2 domain, an FHA domain, a PTB domain, or an antibody. The substrate and detection modules optionally comprise distinct polypeptides.

Exemplary kinases, kinase substrates, and phosphopeptide binding domains have been described above. Essentially all of the other features noted for the embodiments above also apply to these embodiments as well, as relevant: for example, with respect to type of fluorescent label, type of quencher, configuration of the substrate module, attachment of caging groups to the substrate, inclusion of a modulator or potential modulator of the activity of the kinase, inclusion of a second sensor, and/or the like.

The sensors can be used in in vitro assays of enzyme activity. Thus, the composition optionally includes the kinase (e.g., a purified or partially purified kinase) or a cell or tissue lysate (e.g., one including the kinase). The sensor can also be used in in-cell assays of enzyme activity, and the composition thus optionally includes a cell, for example, a cell comprising the sensor and/or the kinase, a nucleic acid encoding the detection module, and/or a nucleic acid encoding the kinase.

Covalent Quenched Enzyme Sensors

In another general class of embodiments in which the quencher is covalently bound to the substrate, use of a detection module is optional. In these embodiments, action of the enzyme on the substrate leads to a conformational change resulting in relief of quenching.

Accordingly, one general class of embodiments provides a composition including a sensor for detecting an activity of a enzyme. The sensor comprises a substrate for the enzyme and a fluorescent label and a quencher covalently connected to the substrate. The substrate is in a first state on which the enzyme can act, thereby converting the substrate to a second state. When the substrate is in the first state, florescent emission by the label is quenched by the quencher. Conversion of the substrate from the first state to the second state alters the net charge of the substrate and results in a conformational change in the sensor that at least partially relieves quenching of the label by the quencher. The intensity of fluorescent emission from the label therefore increases, for example, by at least about 5% (e.g., by at least about 10% or by at least about 20% or more). In one aspect, conversion of the substrate from the first state to the second state introduces an unfavorable intramolecular electrostatic interaction or eliminates a favorable intramolecular electrostatic interaction (e.g., an ionic bond or salt bridge), thereby resulting in the conformational change that relieves quenching.

The substrate can be essentially any suitable substrate, for example, an amino acid, a polypeptide, a nitrogenous base, a nucleoside, a nucleotide, a nucleic acid, a carbohydrate, a lipid, or the like. The substrate is optionally a specific substrate or a generic substrate.

In one class of embodiments, the substrate is a polypeptide substrate. Optionally, conversion of the substrate from the first state to the second state alters the charge of an amino acid side chain in the polypeptide (e.g., as assessed at a relevant pH, e.g., a physiological pH or neutral pH). In one aspect, conversion of the substrate from the first state to the second state involves transfer of a functional group to the side chain. Examples include, but are not limited to, phosphorylation, acetylation, alkylation (e.g., methylation), glycosylation, and sulfation, involving transfer of a phosphoryl, acetyl, alkyl (e.g., methyl), glycosyl, or sulfyl group to the side chain. Similarly, conversion of the substrate from the first state to the second state can involve removal of a functional group from the side chain, for example, dephosphorylation, demethylation, or deacetylation. The functional group that is added or removed from the side chain can be charged or uncharged.

In one class of embodiments, the amino acid side chain in the first state is uncharged and in the second state is negatively charged. Exemplary reactions in this class of embodiments include, but are not limited to, sulfation (e.g., of tyrosine side chains) and phosphorylation (e.g., of serine, threonine, or tyrosine side chains). In one exemplary class of embodiments, the quencher is negatively charged, and conversion of the substrate from the first state in which the amino acid side chain is uncharged to the second state in which the side chain is negatively charged introduces an unfavorable electrostatic interaction between the quencher and the side chain. In a related class of embodiments, one or more amino acid residues adjacent to the quencher are negatively charged, and conversion of the substrate from the first state in which the amino acid side chain is uncharged to the second state in which the side chain is negatively charged introduces an unfavorable electrostatic interaction between the side chain and the residues. The negatively charged residues can be proximal to the quencher in the three-dimensional structure of the polypeptide and/or adjacent to the quencher in the primary structure of the polypeptide.

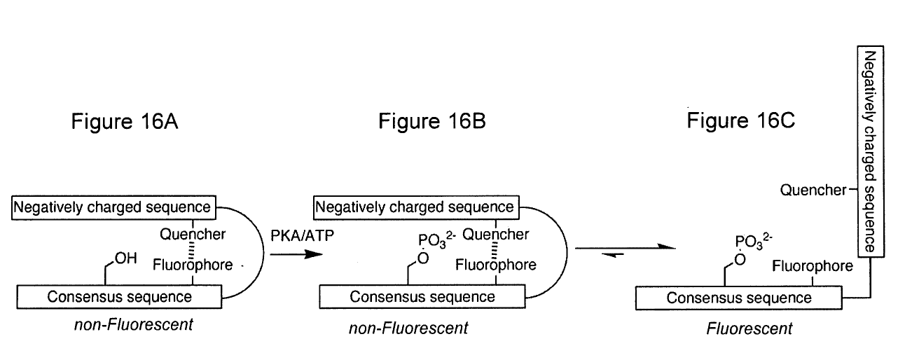

An exemplary embodiment is schematically illustrated in FIG. 16. A serine kinase substrate bearing a fluorophore and quencher, with the substrate in its first, unphosphorylated state, is illustrated in FIG. 16 Panel A. Since the effective concentration of the quencher is high, the quencher and the fluorophore form a ground state complex that significantly reduces or completely eliminates fluorescent behavior. The quencher is surrounded by negatively charged residues; thus, once the serine side chain is phosphorylated (FIG. 16 Panel B), unfavorable electrostatic interactions between the negatively charged phosphoserine and the negatively charged residues surrounding the quencher repel the quencher from the fluorophore, resulting in relief of quenching and restoration of fluorescence (FIG. 16 Panel C). The fluorophore is optionally positioned adjacent to the serine (e.g., on a residue adjacent to the serine).

It will be evident that the quencher can be negatively charged and also adjacent to or flanked by negative amino acid residues, as in exemplary sensors P15-P20, which are described in the Examples sections herein below (see, e.g., Table 7). The composition optionally includes any of P15-P20. More generally, the polypeptide substrate can comprise the amino acid sequence of SEQ ID NO:24 (GRTGRRX⁻¹SLPK⁺³, where X⁻¹ is an amino acid residue comprising a fluorescent label or quencher and where the εN of K⁺³ is modified (e.g., acylated) with a peptide comprising one or more acidic amino acid residues (e.g., D and/or E) and an amino acid residue comprising a quencher or fluorescent label (whichever is not present on X⁻¹)). For example, optionally one or more, two or more, three or more, or four or more of the residue positions immediately N- and/or C-terminal of the position occupied by the quencher can be negatively charged residues such as D and/or E.

In another class of embodiments, the amino acid side chain in the first state is positively charged and in the second state is uncharged. Exemplary reactions in this class of embodiments include, but are not limited to, acetylation of lysine side chains and methylation of lysine or arginine side chains. In one exemplary class of embodiments, the quencher is negatively charged, and conversion of the substrate from the first state in which the amino acid side chain is positively charged to the second state in which the side chain is uncharged eliminates a favorable electrostatic interaction between the quencher and the side chain. In a related class of embodiments, one or more amino acid residues adjacent to the quencher are negatively charged, and conversion of the substrate from the first state in which the amino acid side chain is positively charged to the second state in which the side chain is uncharged eliminates a favorable electrostatic interaction between the side chain and the residues.

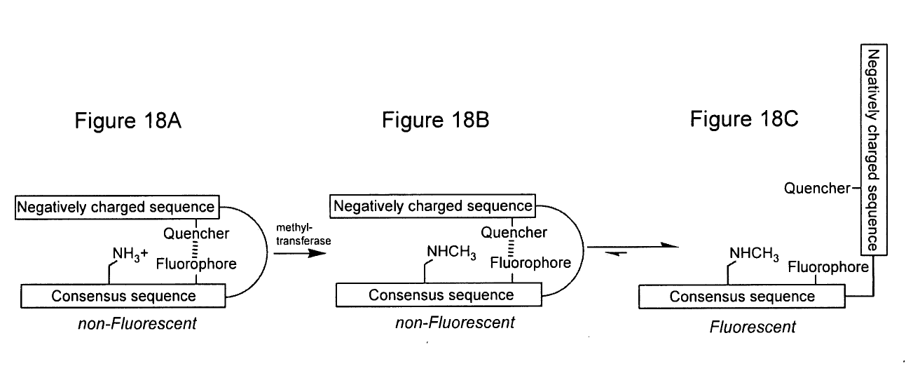

An exemplary embodiment is schematically illustrated in FIG. 18. A lysine methyltransferase substrate bearing a fluorophore and quencher, with the substrate in its first, unmethylated state, is illustrated in FIG. 18 Panel A. The quencher and the fluorophore form a ground state complex that significantly reduces or completely eliminates fluorescent behavior. The quencher is surrounded by one or more negatively charged residues, which experience favorable electrostatic interaction(s) with the positively charged lysine. Once the lysine side chain is methylated, however, the favorable electrostatic interaction(s) no longer occur (FIG. 18 Panel B). Loss of the favorable interaction(s) destabilizes the quenched confirmation of the sensor, leading to a conformational change to a conformation in which the quencher is no longer proximal to the fluorophore and resulting in relief of quenching and restoration of fluorescence (FIG. 18 Panel C).

A variety of other sensor configurations are contemplated and are included in the scope of the appended claims. For example, it will be evident that sensors analogous to those described above can be employed for enzymes such as histidine kinases, where the amino acid side chain in the first state is positively charged and in the second state is negatively charged (or vice versa).

As another example, in the embodiments described above, the fluorescent label is positioned near the residue whose side chain is modified (e.g., adjacent to the residue in the polypeptide's primary structure). Analogous sensors in which the positions of the fluorescent label and the quencher are reversed, such that the quencher rather than the label is near the residue whose side chain is modified, are also contemplated.

As yet another example, the polarity of the charges described above can be reversed. Thus, for example, the quencher (or fluorophore) can be surrounded by positively charged residues rather than negatively charged residues, in embodiments in which the side chain is uncharged (or negatively charged) in the first state and positively charged in the second state (thereby introducing an unfavorable intramolecular electrostatic interaction) or in embodiments in which the side chain is negatively charged in the first state and uncharged in the second state (thereby removing a favorable intramolecular electrostatic interaction).

The sensors can be used to detect activity of any of a large number of enzymes, e.g., in in vitro or in-cell assays. Thus, for example, the enzyme is optionally a protein kinase, a serine/threonine protein kinase, a tyrosine protein kinase, a histone methyltransferase, a histone lysine methyltransferase, a histone arginine methyltransferase, a protein lysine methyltransferase, a histone acetyltransferase, a lysine acetyltransferase, or a protein phosphatase, among many others.

As noted above, conversion of the substrate from the first to the second state optionally involves transfer of a functional group to or removal of a functional group from a side chain of the polypeptide substrate. As other examples, conversion of the substrate from the first state to the second state can involve changing the chemical nature of an amino acid side chain (e.g., conversion of arginine to citrulline by deimination) or modification of the polypeptide termini (e.g., amidation of the C-terminus or acetylation of the N-terminus).

The composition optionally includes a detection module such as those described above, which binds to the substrate when the substrate is in the second state. Use of such a detection module can, in some embodiments, assist in relief of quenching by sequestering the label, amplifying the increase in intensity of fluorescent emission from the label.

Essentially all of the features noted for the embodiments above apply to these embodiments as well, as relevant: for example, with respect to type of fluorescent label, type of quencher, type and configuration of the optional detection module, attachment of caging groups to the substrate, inclusion of a modulator or potential modulator, and/or the like. The composition optionally includes the enzyme, a cell lysate, and/or a cell (e.g., a cell that includes the sensor, the enzyme, a detection module, and/or nucleic acid(s) encoding such detection module or enzyme). It is worth noting that, as for the other embodiments herein, the quencher is typically other than one of the twenty amino acids generally found in naturally occurring polypeptides (e.g., the quencher is typically other than an A, C, D, E, F, G, H, I, K, L, M, N, P, Q, R, S, T, V, W, or Y residue or side chain).

Related Compositions

Other embodiments provide compositions including components of enzyme sensors (e.g., substrate modules, detection modules, and/or quenchers) and/or nucleic acids encoding such components. For example, one general class of embodiments provides a composition that includes a labeled polypeptide, which labeled polypeptide comprises a fluorescent label and a polypeptide that comprises amino acid sequence X⁻⁴R⁻³R⁻²X⁻¹S⁰X⁺¹X⁺², where X⁻⁴ and X⁺² are independently selected from the group consisting of an amino acid residue, an amino acid residue comprising the fluorescent label, and an amino acid residue comprising a quencher, and where X⁻¹ and X⁺¹ are independently selected from the group consisting of a hydrophobic amino acid residue, an amino acid residue comprising the fluorescent label, and an amino acid residue comprising a quencher. S⁰ is optionally unphosphorylated or phosphorylated.

The labeled polypeptide can be essentially any of those described herein. Thus, in one class of embodiments, the polypeptide comprises an amino acid sequence selected from the group consisting of SEQ ID NOs:13-18 and SEQ ID NOs:23-24. For example, the labeled polypeptide can be any of P1-P20.

The composition optionally includes a quencher. In some embodiments, the quencher is covalently connected to the polypeptide (e.g., as in exemplary polypeptides P13-P20). In other embodiments, however, the quencher is not covalently connected to the polypeptide. As just a few examples of compositions including labeled polypeptides and quenchers which are not covalently bound to each other, the composition can include P5 and Rose Bengal, P9 and Aniline Blue WS, P2 and Ponceau S, or P12 and Acid Green 27.

Essentially all of the features noted for the embodiments above apply to these embodiments as well, as relevant: for example, with respect to type of fluorescent label, type of quencher, configuration of the labeled polypeptide, and/or the like. The composition optionally includes a 14-3-3 or similar domain that binds the serine-phosphorylated labeled polypeptide and/or a nucleic acid encoding such a domain, a kinase or phosphatase for which the polypeptide is a substrate and/or a nucleic acid encoding such an enzyme, a cell lysate, and/or a cell (e.g., a cell that includes the labeled polypeptide, a 14-3-3 or similar domain, a kinase or phosphatase, and/or nucleic acid(s) encoding such domain or enzyme).

### Methods for Detecting Enzyme Activity

In one aspect, the invention provides methods for assaying enzyme activity using sensors of the invention. Thus, one general class of embodiments provides methods of assaying an activity of an enzyme. In the methods, the enzyme is contacted with a sensor. The sensor includes 1) a substrate module that comprises a substrate for the enzyme, wherein the substrate is in a first state on which the enzyme can act, thereby converting the substrate to a second state, and a fluorescent label, 2) a detection module, which detection module binds to the substrate module when the substrate is in the first state, or which detection module binds to the substrate module when the substrate is in the second state, and 3) a quencher. In one aspect, the quencher is not covalently bound to the substrate module or to the detection module. Binding of the detection module to the substrate module results in an increased intensity of fluorescent emission from the label. The increased signal from the label is detected and correlated to the activity of the enzyme, thereby assaying the activity of the enzyme.

The assay can be, e.g., qualitative or quantitative. As a few examples, the assay can simply indicate whether the activity is present (e.g., an increase in intensity is detected) or absent (e.g., no signal change is detected), or it can indicate the activity is higher or lower than activity in a corresponding control sample (e.g., the increase in intensity is greater or less than that in a control assay or sample, e.g., one that includes a known quantity of enzyme or premodified substrate or the like), or it can be used to determine a number of activity units of the enzyme (an activity unit is typically defined as the amount of enzyme which will catalyze the transformation of 1 micromole of the substrate per minute under standard conditions).

The methods are optionally used, e.g., for in vitro biochemical assays of enzyme activity using purified or partially purified enzyme, a cell lysate, or the like. Caging the sensor can permit initiation of the activity assay to be precisely controlled, temporally and/or spatially (see, e.g., US patent application publication 2004/0166553). Thus, in one class of embodiments, the sensor comprises one or more caging groups associated with the substrate module (e.g., the substrate), which caging groups inhibit (e.g., prevent) the enzyme from acting upon the substrate. The methods include uncaging the substrate, e.g., by exposing the substrate to uncaging energy, thereby freeing the substrate from inhibition by the one or more caging groups. Typically, the one or more caging groups prevent the enzyme from acting upon the substrate, and removal of or an induced conformational change in the one or more caging groups permits the enzyme to act upon the substrate. The substrate can be uncaged, for example, by exposing the substrate to light of a first wavelength (for photoactivatable or photolabile caging groups), sonicating the substrate module, or otherwise supplying uncaging energy appropriate for the specific caging groups utilized.

Alternatively or in addition, the methods can include uncaging other caged reagents, for example, caged nucleotides (e.g., caged ATP, e.g., to initiate a kinase reaction), caged metal ions, caged chelating agents (e.g., caged EDTA or EGTA, e.g., to terminate a reaction requiring divalent cations), caged activators or inhibitors, or the like.

The methods can include contacting the enzyme with a modulator (e.g., an activator or inhibitor) of its activity. Similarly, the methods can include modulating the activity of at least one other enzyme, e.g., by adding an activator or inhibitor of at least one other enzyme that functions (or potentially functions) in an upstream, downstream, or related signaling or metabolic pathway.

In one aspect, the methods can be used to screen for compounds that affect activity of the enzyme (or binding of the substrate and detection modules to each other). Thus, in one class of embodiments, the methods include contacting the enzyme with a test compound, assaying the activity of the enzyme in the presence of the test compound, and comparing the activity of the enzyme in the presence of the test compound with the activity of the enzyme in the absence of the test compound.

The methods can be used to monitor the activities of two or more enzymes, e.g., in a single reaction mixture. For example, if desired, a second sensor comprising a second substrate module including a second substrate for a second enzyme, a second fluorescent label whose signal is detectably different from that of the first sensor's label, a second detection module, and optionally a second quencher, is contacted with the second enzyme. The second detection module and/or quencher can be the same as or different from the first detection module and/or quencher. An increase in signal from the second label is detected and correlated with the activity of the second enzyme.

Essentially all of the features noted for the compositions above apply to these methods as well, as relevant: for example, with respect to type of enzyme and/or substrate, exemplary substrate and/or detection modules, type of fluorescent label and/or quencher, degree of quenching, fold increase in fluorescence emission, molar ratio of the substrate module to the quencher and/or the detection module, type of caging groups, and/or the like. For example, the quencher can form a non-covalent complex with the substrate module. Binding of the substrate and detection modules disrupts the complex between the quencher and the substrate module, thereby increasing the intensity of fluorescent emission from the label. As for the embodiments above, the non-covalent complex between the quencher and the substrate module optionally has an apparent K_{d} of about 20 μM or less, e.g., about 10 μM or less or even about 1 μM or less.

Another general class of embodiments provides methods of assaying activity of a protein kinase. In the methods, the kinase is contacted with a sensor. The sensor includes a substrate module and a detection module. The substrate module includes a polypeptide substrate for the kinase, wherein the substrate is in a first, unphosphorylated state on which the kinase can act, thereby converting the substrate to a second, phosphorylated state, a fluorescent label, and a quencher. The quencher and typically the label are covalently connected to the substrate. The detection module binds to the substrate module when the substrate is in the second, phosphorylated state. Binding of the detection module to the substrate module results in an increase in intensity of fluorescent emission from the label, preferably, an increase of at least about 1.5 fold (for example, at least about 2 fold, at least about 2.3 fold, or at least about 5 fold or more). The increase in intensity of fluorescent emission from the label is detected and correlated to the activity of the kinase, thereby assaying the activity of the kinase. As for the embodiments above, the assay is optionally qualitative or quantitative.

Essentially all of the features noted for the compositions and methods above apply to these methods as well, as relevant: for example, with respect to type of kinase, exemplary substrate and/or detection modules, type of fluorescent label and/or quencher, caging and uncaging of the sensor, contacting the kinase with a modulator or test compound, and/or the like.

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the sensor comprises introducing the substrate module into a cell. Similarly, in some embodiments, contacting the enzyme and the sensor comprises introducing the detection module into the cell. In other embodiments, the detection module is expressed in the cell, endogenously or exogenously; thus, the methods optionally include introducing a vector encoding the detection module into the cell, whereby the detection module is expressed in the cell. Similarly, the kinase can be expressed endogenously or exogenously in the cell; in one class of embodiments, a vector encoding the kinase is introduced into the cell, whereby the kinase is expressed in the cell.

Yet another general class of embodiments provides methods of assaying an activity of an enzyme. In the methods, the enzyme is contacted with a sensor. The sensor includes a substrate for the enzyme and a fluorescent label and a quencher covalently connected to the substrate. The substrate is in a first state on which the enzyme can act, thereby converting the substrate to a second state. When the substrate is in the first state, florescent emission by the label is quenched by the quencher. Conversion of the substrate from the first state to the second state alters the net charge of the substrate and results in a conformational change in the sensor that at least partially relieves quenching of the label by the quencher, producing an increased intensity of fluorescent emission from the label, e.g., of at least about 5% (e.g., at least about 10% or at least about 20% or more). In one aspect, conversion of the substrate from the first state to the second state introduces an unfavorable intramolecular electrostatic interaction or eliminates a favorable intramolecular electrostatic interaction (e.g., an ionic bond or salt bridge), thereby resulting in the conformational change that relieves quenching. The increased intensity of fluorescent emission from the label is detected and correlated to the activity of the enzyme, thereby assaying the activity of the enzyme. As for the embodiments above, the assay is optionally qualitative or quantitative.

Essentially all of the features noted for the compositions and methods above apply to these methods as well, as relevant: for example, with respect to type of enzyme and/or substrate, configuration of the sensor, exemplary substrates and sensors, type of fluorescent label and/or quencher, caging and uncaging of the substrate, contacting the enzyme with a modulator or test compound, and/or the like. The methods optionally include contacting the substrate with a detection module that binds to the substrate when the substrate is in the second state. Exemplary detection modules have been described above.

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the sensor comprises introducing the sensor into a cell. As for the embodiments above, the cell can express the enzyme and/or optional detection module, endogenously or exogenously.

### Binding Sensors

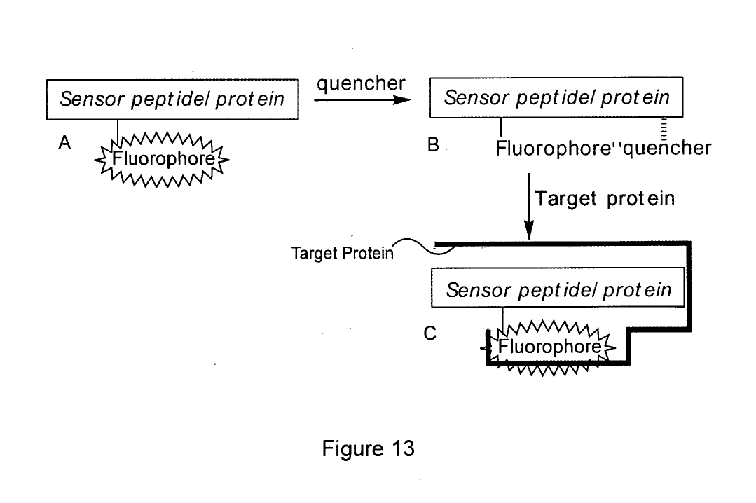

One aspect of the invention provides binding sensors (e.g., combinations of labeled polypeptides and quenchers) for detecting or monitoring an intermolecular association, e.g., between two polypeptides. Accordingly, one general class of embodiments provides a composition including a labeled polypeptide comprising a first polypeptide and a fluorescent label, a second polypeptide to which the first polypeptide binds, and a quencher. Binding of the first polypeptide to the second polypeptide results in an increased intensity of fluorescent emission from the label, since the label is at least partially sequestered from the quencher. In one aspect, the quencher is not covalently bound to the first polypeptide or to the second polypeptide. See, for example, the embodiment schematically illustrated in FIG. 13. In another aspect, the quencher is covalently connected to the first polypeptide.

A wide variety of domains known to recognize various amino acid sequences have been described in the art and can be employed as first or second polypeptides. See, for example, the references above and pawsonlab (dot) mshri (dot) on (dot) ca/index (dot) php?option=com_content&task=view&id=30&Itemid=63. Exemplary domains useful as or in second polypeptides include, but are not limited to, LIM, PDZ, WW, FHA, SH3, 14-3-3, SH2, PTB, chromo-, and bromo-domains.

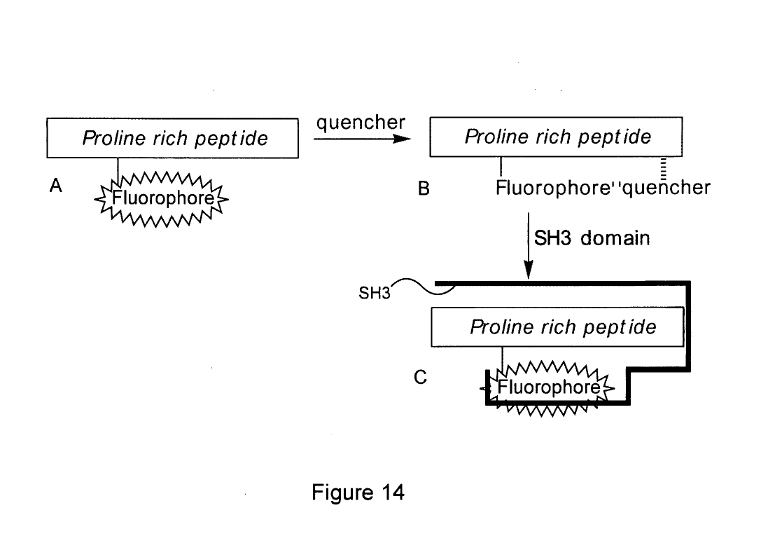

In one exemplary class of embodiments, the first polypeptide is a proline rich polypeptide and the second polypeptide comprises an SH3 domain; see, e.g., the embodiment schematically illustrated in FIG. 14. In another class of embodiments, the first polypeptide comprises a phosphorylated serine residue and the second polypeptide comprises a 14-3-3 domain. In yet another class of embodiments, the first polypeptide comprises a phosphorylated tyrosine residue and the second polypeptide comprises an SH2 or PTB domain. In yet another class of embodiments, the first polypeptide comprises a methylated lysine residue and the second polypeptide comprises a chromodomain, or the first polypeptide comprises an acetylated lysine residue and the second polypeptide comprises a bromodomain.

It will be evident that the substrate modules (or modified forms thereof) and/or detection modules described for the enzyme sensors above can be adapted for use as first and/or second polypeptides in these embodiments. Thus, in one exemplary class of embodiments, the first polypeptide comprises amino acid sequence X⁻⁴R⁻³R⁻²X⁻¹S⁰X⁺¹X⁺², wherein S⁰ is phosphorylated; where X⁻⁴ and X⁺² are independently selected from the group consisting of: an amino acid residue and an amino acid residue comprising the fluorescent label; and where X⁻¹ and X⁺¹ are independently selected from the group consisting of: a hydrophobic amino acid residue and an amino acid residue comprising the fluorescent label. Thus, the first polypeptide optionally comprises an amino acid sequence selected from the group consisting of SEQ ID NOs:13-18, wherein the serine residue is phosphorylated. For example, the labeled polypeptide can be any one of P1-P12 in which the serine residue is phosphorylated, and the second polypeptide optionally comprises a 14-3-3 domain. As a few specific examples, the labeled polypeptide can be serine-phosphorylated P5 and the quencher Rose Bengal, the labeled polypeptide serine-phosphorylated P9 and the quencher Aniline Blue WS, the labeled polypeptide serine-phosphorylated P2 and the quencher Ponceau S, or the labeled polypeptide serine-phosphorylated P12 and the quencher Acid Green 27. In embodiments in which the quencher is covalently connected to the first polypeptide, exemplary labeled polypeptides include any one of P13-P20 in which the serine residue is phosphorylated; the second polypeptide optionally comprises a 14-3-3 domain.

Essentially all of the features noted for the embodiments above apply to these embodiments as well, as relevant: for example, with respect to type of fluorescent label, type of quencher, and/or the like.

For example, it is worth noting that the binding sensors are optionally caged. In one class of embodiments, the sensor is caged such that the first and second polypeptides can not bind to each other until the sensor is uncaged, for example, by removal of a photolabile caging group. Thus, in one class of embodiments, the labeled polypeptide comprises one or more caging groups associated with the first polypeptide. The caging groups inhibit the first polypeptide from binding to the second polypeptide, e.g., by at least about 75%, at least about 90%, at least about 95%, or at least about 98%, as compared to binding in the absence of the one or more caging groups. Preferably, the one or more caging groups prevent the first polypeptide from binding to the second polypeptide. Typically, removal of, or an induced conformational change in, the one or more caging groups permits the first polypeptide to bind to the second polypeptide. The one or more caging groups associated with the first polypeptide can be covalently or non-covalently attached to polypeptide. In a preferred aspect, the one or more caging groups are photoactivatable (e.g., photolabile). For example, in one embodiment, the labeled polypeptide comprises one or more photolabile caging groups covalently bound to the first polypeptide, which caging groups inhibit or prevent the first polypeptide from binding to the second polypeptide. As noted above, caging groups are described in greater detail below, in the section entitled “Caging groups”.

The increase in signal from the fluorescent label upon binding of the first and second polypeptides is optionally an increase of at least about 7 fold, at least about 10 fold, at least about 20 fold, at least about 50 fold, at least about 60 fold, at least about 100 fold, or at least about 200 fold.

Preferably, when the labeled first polypeptide is not bound to the second polypeptide, the label exhibits little or no fluorescence. Thus, in one aspect, when the first polypeptide is not bound to the second polypeptide, the quencher quenches fluorescent emission by the label by at least about 40%, as compared to fluorescent emission in the absence of the quencher. For example, the quencher can quench fluorescent emission by the label by at least about 50%, at least about 75%, at least about 90%, or at least about 95%, or can even prevent detectable emission from the label, e.g., at a given wavelength.

The quencher can quench fluorescent emission from the label when the label and quencher are in proximity, e.g., in solution. In one aspect, the quencher forms a non-covalent complex with the labeled polypeptide, putting the quencher in proximity to the label. The complex is stabilized by non-covalent interactions between the quencher and the label and/or first polypeptide, for example, by electrostatic interactions, hydrophobic interactions, and/or hydrogen bonds between the quencher and the label and/or first polypeptide (e.g., by electrostatic interactions between a negatively charged moiety on the quencher and positively charged side chain(s) on the first polypeptide and/or by hydrophobic interactions between the quencher and the label). Binding of the second polypeptide to the labeled polypeptide disrupts the interactions between the quencher and the labeled polypeptide, disrupting the complex between the quencher and the labeled polypeptide and thereby increasing the intensity of fluorescent emission from the label. In one class of embodiments, the non-covalent complex between the quencher and the labeled polypeptide has an apparent dissociation constant (apparent K_{d}) of about 20 μM or less, e.g., about 10 μM or less or even about 1 μM or less.

The molar ratio of the quencher to the labeled polypeptide in the composition can be varied, e.g., to achieve a desired level of quenching in the absence of binding of the first polypeptide to the second polypeptide. For example, the molar ratio of the quencher to the labeled polypeptide in the composition can be at least about 1 to 1, at least about 5 to 1, at least about 10 to 1, at least about 25 to 1, or at least about 50 to 1.

The binding sensors can be used to study the effects of compounds that affect (potentiate or inhibit) or potentially affect the interaction between the first and second polypeptides. Thus, the composition optionally includes an inhibitor or potential inhibitor of the interaction between the first and second polypeptides, for example, a compound that competes with the first polypeptide for binding to the second polypeptide or a compound that noncompetitively inhibits binding of the first polypeptide to the second polypeptide.

A second binding sensor (e.g., including a second, detectably different label) is optionally included in the composition to monitor an additional protein-protein interaction. Other embodiments provide compositions including components of the binding sensor compositions (e.g., first polypeptides, quenchers, and/or second polypeptides) and/or nucleic acids encoding such components.

### Methods for Assaying Protein-Protein Interactions

One general class of embodiments provides methods of assaying an intermolecular interaction between a first polypeptide and a second polypeptide. In the methods, a labeled polypeptide comprising the first polypeptide and a fluorescent label is provided, as is a quencher. In one aspect, the quencher is not covalently bound to the first polypeptide or to the second polypeptide. The labeled polypeptide, the quencher, and the second polypeptide are contacted, thereby permitting the first polypeptide to bind to the second polypeptide. Binding of the first polypeptide to the second polypeptide results in an increased intensity of fluorescent emission from the label. The increased intensity of fluorescent emission is detected and correlated to binding of the first and second polypeptides.

The assay can be, e.g., qualitative or quantitative. As a few examples, the assay can simply indicate whether the protein-protein interaction occurs (e.g., an increase in intensity is detected) or does not occur (e.g., no signal change is detected), or it can indicate the extent to which the interaction occurs as compared to a corresponding control sample (e.g., the increase in intensity is greater or less than that in a control assay or sample, e.g., one that includes a known quantity of second polypeptide), or it can be used to quantitate the interaction in some way (e.g., to determine a K_{d} for the protein-protein complex).

The methods are optionally used, e.g., for in vitro biochemical assays of intermolecular interactions using purified or partially purified enzyme, a cell lysate, or the like. As for the embodiments above, caging the binding sensor can permit initiation of the assay to be precisely controlled, temporally and/or spatially. Thus, in one class of embodiments, the labeled polypeptide comprises one or more caging groups associated with the first polypeptide, which caging groups inhibit (e.g., prevent) the first polypeptide from binding to the second polypeptide. The methods include uncaging the first polypeptide, e.g., by exposing the first polypeptide to uncaging energy, thereby freeing the first polypeptide from inhibition by the one or more caging groups. Typically, the one or more caging groups prevent the first polypeptide from binding to the second polypeptide, and removal of or an induced conformational change in the one or more caging groups permits the first polypeptide to bind to the second polypeptide. The first polypeptide can be uncaged, for example, by exposing it to light of a first wavelength (for photoactivatable or photolabile caging groups), sonicating it, or otherwise supplying uncaging energy appropriate for the specific caging groups utilized.

The methods can be used to monitor the interaction of two or more sets of molecules, e.g., in a single reaction mixture, by using a second binding sensor. The methods can include contacting the enzyme with a compound that affects (potentiates or inhibits) or potentially affects the interaction between the first and second polypeptides.

In one aspect, the methods can be used to screen for compounds (e.g., synthetic peptides, small molecules, etc.) that affect the interaction between the first and second polypeptides. Thus, in one class of embodiments, the methods include contacting the second polypeptide with a test compound, assaying the interaction between the first and second polypeptides in the presence of the test compound, and comparing the interaction between the first and second polypeptides in the presence of the test compound with interaction between the first and second polypeptides in the absence of the test compound. The test compound is optionally one that inhibits binding of the first and second polypeptides, for example, a compound that competes with the first polypeptide for binding to the second polypeptide. For example, the test compound is optionally a compound (e.g., a synthetic peptide) that binds to a 14-3-3, SH2, SH3, PTB, chromo-, or bromo-domain.

As just one example, the methods can be used in a screen to identify inhibitory ligands for 14-3-3 proteins. High fluorescence is observed when a suitable labeled polypeptide and a quencher (e.g., one of the combinations described herein, such as serine-phosphorylated P5 and Rose Bengal, serine-phosphorylated P9 and Aniline Blue WS, serine-phosphorylated P2 and Ponceau S, or serine-phosphorylated P12 and Acid Green 27) are contacted with a second polypeptide including a 14-3-3 domain. Screening through a library of potential 14-3-3 inhibitory ligands can be conducted simply by contacting each member of the library (singly or in combination) with the labeled polypeptide, quencher, and second polypeptide; promising compounds (inhibitory ligands) generate a drop in fluorescent intensity, typically, a substantial decrease in or even elimination of observed fluorescence. Such inhibitors are of interest, for example, as therapeutic agents to block signaling through 14-3-3-mediated pathways involved in diseases such as cancer. See, e.g., Wilker and Yaffe (2004) “14-3-3 proteins—a focus on cancer and human disease” J Mol Cell Cardiol 37:633-642.

Essentially all of the features noted for the embodiments above apply to these methods as well, as relevant: for example, with respect to exemplary first and/or second polypeptides, type of fluorescent label and/or quencher, degree of quenching, fold increase in fluorescence emission, molar ratio of the labeled polypeptide to the quencher and/or the second polypeptide, and/or the like. For example, the quencher can form a non-covalent complex with the labeled polypeptide. Binding of the first and second polypeptides disrupts the complex between the quencher and the labeled polypeptide, thereby increasing the intensity of fluorescent emission from the label. As for the embodiments above, the non-covalent complex between the quencher and the labeled polypeptide optionally has an apparent K_{d} of about 20 μM or less, e.g., about 10 μM or less or even about 1 μM or less.

### Kits

Kits comprising components of compositions of the invention and/or that can be used in practicing the methods of the invention form another feature of the invention. For example, in one class of embodiments, the kit includes a sensor for detecting an activity of an enzyme, packaged in one or more containers. The sensor comprises a substrate module, a detection module, and a quencher. The substrate module includes a substrate for the enzyme, wherein the substrate is in a first state on which the enzyme can act, thereby converting the substrate to a second state, and a fluorescent label. The detection module binds to the substrate module when the substrate is in the first state or when the substrate is in the second state. Binding of the detection module to the substrate module results in an increased intensity of fluorescent emission from the label, since the label is at least partially sequestered from the quencher. In one aspect, the quencher is not covalently bound to the substrate module or to the detection module. Typically, the kit also includes instructions for using the sensor to detect the activity of the enzyme. The kit optionally also includes one or more buffers, controls including a known quantity of the enzyme, and/or the like. Essentially all of the features noted for the compositions above apply to these kits as well, as relevant: for example, with respect to type of enzyme, exemplary substrate and/or detection modules, type of fluorescent label and/or quencher, inclusion of caging groups, and/or the like.

Another class of embodiments provides a kit that includes a sensor for detecting an activity of a protein kinase, packaged in one or more containers. The sensor comprises a substrate module and a detection module. The substrate module includes a polypeptide substrate for the kinase, wherein the substrate is in a first, unphosphorylated state on which the kinase can act, thereby converting the substrate to a second, phosphorylated state, a fluorescent label, and a quencher. The quencher and typically the label are covalently connected to the substrate. The detection module binds to the substrate module when the substrate is in the second, phosphorylated state. Binding of the detection module to the substrate module results in an increase in intensity of fluorescent emission from the label, since the label is at least partially sequestered from the quencher. Preferably, the increase in intensity is an increase of at least about 1.5 fold, for example, at least about 2 fold, at least about 2.3 fold, or at least about 5 fold or more. Optionally, the kit includes the substrate module and not the detection module, for example, for detection of kinase activity in cells expressing a suitable detection module. Typically, the kit also includes instructions for using the sensor to detect the activity of the kinase. The kit optionally also includes one or more buffers, controls including a known quantity of the kinase, and/or the like. Essentially all of the features noted for the compositions above apply to these kits as well, as relevant: for example, with respect to type of enzyme, exemplary substrate and/or detection modules, type of fluorescent label and/or quencher, inclusion of caging groups, and/or the like. One or more reagents for introducing the sensor or components thereof (e.g., the substrate module) into a cell are optionally included in the kit.

Yet another class of embodiments provides a kit that includes a sensor for detecting an activity of a enzyme. The sensor comprises a substrate for the enzyme and a fluorescent label and a quencher covalently connected to the substrate. The substrate is in a first state on which the enzyme can act, thereby converting the substrate to a second state. When the substrate is in the first state, florescent emission by the label is quenched by the quencher. Conversion of the substrate from the first state to the second state alters the net charge of the substrate (e.g., introducing an unfavorable intramolecular electrostatic interaction or eliminating a favorable intramolecular electrostatic interaction) and results in a conformational change in the sensor that at least partially relieves quenching of the label by the quencher. Typically, the kit also includes instructions for using the sensor to detect the activity of the enzyme. The kit optionally also includes a detection module, one or more buffers, controls including a known quantity of the enzyme, and/or the like. Essentially all of the features noted for the compositions above apply to these kits as well, as relevant: for example, with respect to type of enzyme, exemplary substrate and/or detection modules, type of fluorescent label and/or quencher, inclusion of caging groups, and/or the like. One or more reagents for introducing the sensor into a cell are optionally included in the kit.

In another class of embodiments, a kit includes a sensor for detecting or monitoring an intermolecular association, e.g., between two polypeptides. The kit includes a quencher and a labeled polypeptide comprising a first polypeptide and a fluorescent label, packaged in one or more containers. The first polypeptide is capable of binding to a second polypeptide, where binding of the first polypeptide to the second polypeptide results in an increased intensity of fluorescent emission from the label, since the label is at least partially sequestered from the quencher. In one aspect, the quencher is not covalently bound to the first polypeptide or to the second polypeptide. Typically, the kit also includes instructions for using the sensor to assay the protein-protein interaction. The kit optionally also includes one or more buffers, controls including a known quantity of the second polypeptide, and/or the like. Essentially all of the features noted for the compositions above apply to these kits as well, as relevant: for example, with respect to exemplary first and/or second polypeptides, type of fluorescent label and/or quencher, inclusion of caging groups, and/or the like.

### Systems

In one aspect, the invention includes systems, e.g., systems used to practice the methods herein and/or comprising the compositions described herein. The system can include, e.g., a fluid handling element, a fluid containing element, a laser for exciting a fluorescent label, a detector for detecting a signal from a label (e.g., fluorescent emissions from a fluorescent label), a source of uncaging energy for uncaging caged sensors, and/or a robotic element that moves other components of the system from place to place as needed (e.g., a multiwell plate handling element). For example, in one class of embodiments, a composition of the invention is contained in a microplate reader or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element, and/or laser). The computer can also receive data from other components of the system, e.g., from a detector, and can interpret the data (e.g., by correlating a change in signal from the label with an activity of an enzyme or with a protein-protein interaction), provide it to a user in a human readable format, or use that data to initiate further operations, in accordance with any programming by the user.

### Fluorescent Labels

As noted, the various sensors and labeled polypeptides of this invention include fluorescent labels. A wide variety of fluorescent labels have been described in the art and can be adapted to the practice of the present invention. Examples include, but are not limited to, dapoxyl, NBD, Cascade Yellow, dansyl, PyMPO, pyrene, 7-diethylaminocoumarin-3-carboxylic acid, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. For example, various derivatives of coumarin are described in Section 1.7 of The Handbook—A Guide to Fluorescent Probes and Labeling Technologies, Tenth Edition, available on the internet at probes (dot) invitrogen (dot) com/handbook; exemplary coumarin derivatives include, but are not limited to, aminocoumarins, hydroxycoumarins, methoxycoumarins, 7-diethylaminocoumarin-3-carboxylic acid, Alexa Fluor 350, Alexa Fluor 430, Marina Blue™, and Pacific Blue™. Fluorescent labels employed in the invention are optionally small molecules, e.g., having a molecular weight of less than about 1000 daltons.

The labels are optionally environmentally sensitive or environmentally insensitive labels. Environmentally insensitive labels are preferred in certain embodiments, since such labels typically provide brighter emissions. The fluorescence of an environmentally insensitive fluorescent label is typically not significantly affected by the solvent in which the label is located. For example, the signal from an environmentally insensitive fluorescent label is typically not significantly different whether the label is in an aqueous solution, a less polar solvent (e.g., methanol), or a nonpolar solvent (e.g., hexane). In contrast, the signal from an environmentally sensitive label changes when the environment of the label changes. For example, the fluorescence of an environmentally sensitive fluorescent label changes when the hydrophobicity, pH, and/or the like of the label's environment changes (e.g., upon binding of the substrate module with which the label is associated to a detection module, such that the label is transferred from an aqueous environment to a more hydrophobic environment at the binding interface between the modules). Typically, the signal from an environmentally sensitive label is affected by the solvent in which the label is located. For example, the signal from an environmentally sensitive fluorescent label is typically significantly different when the label is in an aqueous solution versus in a less polar solvent (e.g., methanol) versus in a nonpolar solvent (e.g., hexane). Examples of environmentally sensitive fluorophores include, but are not limited to, those described in U.S. patent application Ser. No. 11/366,221 and references therein, including in US patent application publication 20020055133 by Hahn et al. entitled “Labeled peptides, proteins and antibodies and processes and intermediates useful for their preparation.”

Signals from the fluorescent labels can be detected by essentially any method known in the art (e.g., fluorescence spectroscopy, fluorescence microscopy, etc.). Excitation and emission wavelengths for the exemplary fluorophores described above can be found, e.g., in The Handbook—A Guide to Fluorescent Probes and Labeling Technologies, Tenth Edition, available on the internet at probes (dot) invitrogen (dot) com/handbook, and in the references above.

Labels (or, similarly, quenchers) can be attached to molecules (e.g., substrates) during synthesis or by postsynthetic reactions by techniques established in the art. For example, a fluorescently labeled nucleotide can be incorporated into a nucleic acid during enzymatic or chemical synthesis of the nucleic acid, e.g., at a preselected or random nucleotide position. Alternatively, fluorescent labels can be added to nucleic acids by postsynthetic reactions, at either random or preselected positions (e.g., an oligonucleotide can be chemically synthesized with a terminal amine or free thiol at a preselected position, and a fluorophore can be coupled to the oligonucleotide via reaction with the amine or thiol). Reactive forms of various fluorophores are commercially available e.g., from Molecular Probes, Inc., or can readily be prepared by one of skill in the art and used for incorporation of the labels into desired molecules. As another example, a fluorescently labeled residue can be incorporated into a polypeptide during enzymatic or chemical synthesis of the polypeptide. Alternatively, fluorescent labels can be added to polypeptides by postsynthetic reactions. A polypeptide substrate optionally comprises one or more residues incorporated to facilitate attachment of the label, e.g., an (L)-2,3-diaminopropionic acid (Dap), (L)-2,4-diaminobutyric acid (Dab), ornithine, lysine, cysteine, or homocysteine residue (or essentially any other chemically reactive natural or unnatural amino acid derivative or residue) to which the label is attached. See, e.g., the Examples sections herein, and U.S. patent application Ser. No. 11/366,221 and US patent application publication 20020055133.

### Caging Groups

A large number of caging groups, and a number of reactive compounds that can be used to covalently attach caging groups to other molecules, are well known in the art. Examples of photolabile caging groups include, but are not limited to: nitroindolines; N-acyl-7-nitroindolines; phenacyls; hydroxyphenacyl; brominated 7-hydroxycoumarin-4-ylmethyls (e.g., Bhc); benzoin esters; dimethoxybenzoin; meta-phenols; 2-nitrobenzyl; 1-(4,5-dimethoxy-2-nitrophenyl)ethyl (DMNPE); 4,5-dimethoxy-2-nitrobenzyl (DMNB); alpha-carboxy-2-nitrobenzyl (CNB); 1-(2-nitrophenyl)ethyl (NPE); 5-carboxymethoxy-2-nitrobenzyl (CMNB); (5-carboxymethoxy-2-nitrobenzyl)oxy) carbonyl; (4,5-dimethoxy-2-nitrobenzyl)oxy) carbonyl; desoxybenzoinyl; and the like. See, e.g., U.S. Pat. No. 5,635,608 to Haugland and Gee (Jun. 3, 1997) entitled “α-carboxy caged compounds”; Neuro 19, 465 (1997); J Physiol 508.3, 801 (1998); Proc Natl Acad Sci USA 1988 September, 85(17):6571-5; J Biol Chem 1997 Feb. 14, 272(7):4172-8; Neuron 20, 619-624, 1998; Nature Genetics, vol. 28:2001:317-325; Nature, vol. 392, 1998:936-941; Pan, P., and Bayley, H. “Caged cysteine and thiophosphoryl peptides” FEBS Letters 405:81-85 (1997); Pettit et al. (1997) “Chemical two-photon uncaging: a novel approach to mapping glutamate receptors” Neuron 19:465-471; Furuta et al. (1999) “Brominated 7-hydroxycoumarin-4-ylmethyls: novel photolabile protecting groups with biologically useful cross-sections for two photon photolysis” Proc. Natl. Acad. Sci. 96(4):1193-1200; Zou et al. “Catalytic subunit of protein kinase A caged at the activating phosphothreonine” J. Amer. Chem. Soc. (2002) 124:8220-8229; Zou et al. “Caged Thiophosphotyrosine Peptides” Angew. Chem. Int. Ed. (2001) 40:3049-3051; Conrad II et al. “p-Hydroxyphenacyl Phototriggers: The reactive Excited State of Phosphate Photorelease” J. Am. Chem. Soc. (2000) 122:9346-9347; Conrad II et al. “New Phototriggers 10: Extending the π,π* Absorption to Release Peptides in Biological Media” Org. Lett. (2000) 2:1545-1547; Givens et al. “A New Phototriggers 9: p-Hydroxyphenacyl as a C-Terminus Photoremovable Protecting Group for Oligopeptides” J. Am. Chem. Soc. (2000) 122:2687-2697; Bishop et al. “40-Aminomethyl-2,20-bipyridyl-4-carboxylic Acid (Abc) and Related Derivatives: Novel Bipyridine Amino Acids for the Solid-Phase Incorporation of a Metal Coordination Site Within a Peptide Backbone” Tetrahedron (2000) 56:4629-4638; Ching et al. “Polymers As Surface-Based Tethers with Photolytic triggers Enabling Laser-Induced Release/Desorption of Covalently Bound Molecules” Bioconjugate Chemistry (1996) 7:525-8; BioProbes Handbook, 2002 from Molecular Probes, Inc.; and The Handbook—A Guide to Fluorescent Probes and Labeling Technologies, Tenth Edition (2005) or Web Edition, from Invitrogen, Inc./Molecular Probes as well as the references below. Many compounds, kits, etc. for use in caging various molecules are commercially available, e.g., from Invitrogen, Inc./Molecular Probes (www (dot) molecularprobes (dot) com).

Environmentally responsive polymers suitable for use as caging groups have also been described. Such polymers undergo conformational changes induced by light, an electric or magnetic field, a change in pH and/or ionic strength, temperature, or addition of an antigen or saccharide, or other environmental variables. For example, Shimoboji et al. (2002) “Photoresponsive polymer-enzyme switches” Proc. Natl. Acad. Sci. USA 99:16,592-16,596 describes polymers that undergo reversible conformational changes in response to light. Such polymers can, e.g., be used as photoactivatable caging groups. See US patent application publication 2004/0166553. See also Ding et al. (2001) “Size-dependent control of the binding of biotinylated proteins to streptavidin using a polymer shield” Nature 411:59-62; Miyata et al. (1999) “A reversibly antigen-responsive hydrogel” Nature 399:766-769; Murthy et al. (2003) “Bioinspired pH-responsive polymers for the intracellular delivery of biomolecular drugs” Bioconjugate Chem. 14:412-419; and Galaev and Mattiasson (1999) “‘Smart’ polymers and what they could do in biotechnology and medicine” Trends Biotech. 17:335-340.

An alternative method for caging a molecule is to enclose the molecule in a photolabile vesicle (e.g., a photolabile lipid vesicle), optionally including a protein transduction domain or the like. Similarly, the molecule can be loaded into the pores of a porous bead which is then encased in a photolabile gel. As another alternative, a caging group optionally comprises a first binding moiety that can bind to a second binding moiety. For example, the caging group can include a biotin (the first binding moiety in this example); a second binding moiety, e.g., streptavidin or avidin, can thus be bound to the caging group, increasing its bulkiness and its effectiveness at caging. In certain embodiments, a caged component comprises two or more caging groups each comprising a first binding moiety, and the second binding moiety can bind two or more first binding moieties simultaneously. See US patent application publication 2004/0166553.

Caged polypeptides (including, e.g., polypeptide substrates, substrate modules, and detection modules) can be produced, e.g., by reacting a polypeptide with a caging compound or by incorporating a caged amino acid during synthesis of a polypeptide. See, e.g., Tatsu et al. (1996) “Solid-phase synthesis of caged peptides using tyrosine modified with a photocleavable protecting group: Application to the synthesis of caged neuropeptide Y” Biochem Biophys Res Comm 227:688-693, which describes synthesis of polypeptides including tyrosine residues caged with 2-nitrobenzyl groups; Veldhuyzen et al. (2003) “A light-activated probe of intracellular protein kinase activity” J Am Chem Soc 125:13358-9, which describes synthesis of a polypeptide including a caged serine; and Vazquez et al. (2003) “Fluorescent caged phosphoserine peptides as probes to investigate phosphorylation-dependent protein associations” J. Am. Chem. Soc. 125:10150-10151, which describes synthesis of a polypeptide including a caged phosphoserine. See also, e.g., U.S. Pat. No. 5,998,580 to Fay et al. (Dec. 7, 1999) entitled “Photosensitive caged macromolecules”; Kossel et al. (2001) PNAS 98:14702-14707; Trends Plant Sci (1999) 4:330-334; PNAS (1998) 95:1568-1573; J Am Chem Soc (2002) 124:8220-8229; Pharmacology & Therapeutics (2001) 91:85-92; and Angew Chem Int Ed Engl (2001) 40:3049-3051. A photolabile polypeptide linker can, for example, comprise a photolabile amino acid such as that described in U.S. Pat. No. 5,998,580, supra. Polypeptides can be caged at backbone and/or side chain nitrogens as described in U.S. patent application 60/876,297 entitled “Photosensitive polypeptides and methods of their production and use” by Lawrence.

Caged nucleic acids (e.g., DNA, RNA or PNA) can be produced by reacting the nucleic acids with caging compounds or by incorporating a caged nucleotide during synthesis of a nucleic acid. See, e.g., U.S. Pat. No. 6,242,258 to Haselton and Alexander (Jun. 5, 2001) entitled “Methods for the selective regulation of DNA and RNA transcription and translation by photoactivation”; Nature Genetics (2001) 28: 317-325; and Nucleic Acids Res. (1998) 26:3173-3178.

Caged modulators (e.g., inhibitors and activators), small molecules, etc. can be similarly produced by reaction with caging compounds or by synthesis. See, e.g., Trends Plant Sci (1999) 4:330-334; PNAS (1998) 95:1568-1573; U.S. Pat. No. 5,888,829 to Gee and Millard (Mar. 30, 1999) entitled “Photolabile caged ionophores and method of using in a membrane separation process”; U.S. Pat. No. 6,043,065 to Kao et al. (Mar. 28, 2000) entitled “Photosensitive organic compounds that release 2,5,-di(tert-butyl)hydroquinone upon illumination”; U.S. Pat. No. 5,430,175 to Hess et al. (Jul. 4, 1995) entitled “Caged carboxyl compounds and use thereof”; U.S. Pat. No. 5,872,243; and PNAS (1980) 77:7237-41. A number of caged compounds, including for example caged nucleotides, caged Ca2+, caged chelating agents, caged neurotransmitters, and caged luciferin, are commercially available, e.g., from Molecular Probes, Inc. (on the world wide web at molecularprobes (dot) com).

Useful site(s) of attachment of caging groups to a given molecule can be determined by techniques known in the art. For example, a molecule with a known activity can be reacted with a caging compound. The resulting caged molecule can then be tested to determine if its activity is sufficiently abrogated. As another example, amino acid residues central to the activity of a polypeptide substrate (e.g., a residue modified by the enzyme, the backbone amide of a residue modified by the enzyme or an adjacent residue, residues located at a binding interface, or the like) can be identified by routine techniques such as scanning mutagenesis, sequence comparisons and site-directed mutagenesis, or the like. Such residues can then be caged, and the activity of the caged substrate can be assayed to determine the efficacy of caging.

Appropriate methods for uncaging caged molecules are also known in the art. For example, appropriate wavelengths of light for removing many photolabile groups have been described; e.g., 300-360 nm for 2-nitrobenzyl, 350 nm for benzoin esters, and 740 nm for brominated 7-hydroxycoumarin-4-ylmethyls (two-photon) (see, e.g., references herein). Conditions for uncaging any caged molecule (e.g., the optimal wavelength for removing a photolabile caging group) can be determined according to methods well known in the art. Instrumentation and devices for delivering uncaging energy are likewise known (e.g., sonicators, heat sources, light sources, and the like). For example, well-known and useful light sources include e.g., a lamp, a laser (e.g., a laser optically coupled to a fiber-optic delivery system) or a light-emitting compound. See also U.S. patent application Ser. No. 10/716,176 by Witney et al. entitled “Uncaging devices.”

### Molecular Biological Techniques

In practicing the present invention, many conventional techniques in molecular biology, microbiology, and recombinant DNA technology are optionally used (e.g., for making and/or manipulating nucleic acids, polypeptides, and/or cells of the invention). These techniques are well known, and detailed protocols for numerous such procedures (including, e.g., in vitro amplification of nucleic acids, cloning, mutagenesis, transformation, cellular transduction with nucleic acids, protein expression, and/or the like) are described in, for example, Berger and Kimmel, Guide to Molecular Cloning Techniques Methods in Enzymology volume 152 Academic Press, Inc., San Diego, Calif.; Sambrook et al., Molecular Cloning—A Laboratory Manual (3rd Ed.), Vol. 1-3, Cold Spring Harbor Laboratory, Cold Spring Harbor, N.Y., 2002; and Current Protocols in Molecular Biology, F. M. Ausubel et al., eds., Current Protocols, a joint venture between Greene Publishing Associates, Inc. and John Wiley & Sons, Inc., (supplemented through 2008)). Other useful references, e.g. for cell isolation and culture include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. A variety of vectors, including expression vectors, have been described and are readily available to one of skill, as are a large number of cells and cell lines suitable for the maintenance and use of such vectors.

### Polypeptide Production

Polypeptides (e.g., polypeptide substrates, detection modules, substrate modules, etc.) can optionally be produced by expression in a host cell transformed with a vector comprising a nucleic acid encoding the desired polypeptide(s). Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography, for example. Protein refolding steps can be used, as desired, in completing configuration of the mature protein. Finally, high performance liquid chromatography (HPLC) can be employed in the final purification steps. See, e.g., the references noted above and Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods 2^{nd} Edition Wiley-Liss, NY; Walker (1996) The Protein Protocols Handbook Humana Press, NJ; Harris and Angal (1990) Protein Purification Applications: A Practical Approach IRL Press at Oxford, Oxford, U.K.; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

Alternatively, cell-free transcription/translation systems can be employed to produce polypeptides encoded by nucleic acids. A number of suitable in vitro transcription and translation systems are commercially available. A general guide to in vitro transcription and translation protocols is found in Tymms (1995) In vitro Transcription and Translation Protocols Methods in Molecular Biology Volume 37, Garland Publishing, NY.

In addition, polypeptides (including, e.g., polypeptides comprising fluorophores and/or unnatural amino acids) can be produced manually or by using an automated system, by direct peptide synthesis using solid-phase techniques (see, e.g., Chan and White, Eds., (2000) Fmoc Solid Phase Peptide Synthesis: A Practical Approach, Oxford University Press, New York, N.Y.; Lloyd-Williams, P. et al. (1997) Chemical Approaches to the Synthesis of Peptides and Proteins, CRC Press; Stewart et al. (1969) Solid-Phase Peptide Synthesis, WH Freeman Co, San Francisco; Merrifield J (1963) J. Am. Chem. Soc. 85:2149-2154; see also the Examples section herein). Exemplary automated systems include the Applied Biosystems 431A Peptide Synthesizer (Perkin Elmer, Foster City, Calif.). In addition, there are many commercial providers of peptide synthesis services. If desired, subsequences can be chemically synthesized separately, and combined using chemical methods to provide full-length polypeptides.

### Production of Aptamers and Antibodies

Aptamers can be selected, designed, etc. for binding various ligands (e.g., substrates in a first or second state) by methods known in the art. For example, aptamers are reviewed in Sun S. “Technology evaluation: SELEX, Gilead Sciences Inc.” Curr Opin Mol. Ther. 2000 February; 2(1):100-5; Patel D J, Suri A K. “Structure, recognition and discrimination in RNA aptamer complexes with cofactors, amino acids, drugs and aminoglycoside antibiotics” J. Biotechnol. 2000 March, 74(1):39-60; Brody E N, Gold L. “Aptamers as therapeutic and diagnostic agents” J. Biotechnol. 2000 March, 74(1):5-13; Hermann T, Patel D J. “Adaptive recognition by nucleic acid aptamers” Science 2000 Feb. 4, 287(5454):820-5; Jayasena S D. “Aptamers: an emerging class of molecules that rival antibodies in diagnostics” Clin Chem. 1999 September, 45(9):1628-50; and Famulok M, Mayer G. “Aptamers as tools in molecular biology and immunology” Curr Top Microbiol Immunol. 1999, 243:123-36.

Antibodies, e.g., that recognize the first or second state of a substrate, can likewise be generated by methods known in the art. For the production of antibodies to a particular polypeptide (e.g., for use as a detection module), various host animals may be immunized by injection with the polypeptide or a portion thereof. Such host animals include, but are not limited to, rabbits, mice and rats, to name but a few. Various adjuvants may be used to enhance the immunological response, depending on the host species; adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a protein or an antigenic functional derivative thereof. For the production of polyclonal antibodies, host animals, such as those described above, may be immunized by injection with the protein, or a portion thereof, supplemented with adjuvants as also described above. The protein can optionally be produced and purified as described herein. For example, recombinant protein can be produced in a host cell, or a synthetic peptide derived from the sequence of the protein can be conjugated to a carrier protein and used as an immunogen. Standard immunization protocols are described in, e.g., Harlow and Lane (1988) Antibodies, A Laboratory Manual, Cold Spring Harbor Publications, New York. Additional references and discussion of antibodies is also found herein.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al. (1983) Immunology Today 4:72; Cole et al. (1983) Proc. Natl. Acad. Sci. USA 80:2026-2030), and the EBV-hybridoma technique (Cole et al. (1985) Monoclonal Antibodies and Cancer Therapy, Alan R. Liss, Inc., pp. 77-96). Such antibodies may be of any immunoglobulin class, including IgG, IgM, IgE, IgA, IgD, and any subclass thereof. The hybridoma producing the mAb of this invention may be cultivated in vitro or in vivo.

In addition, techniques developed for the production of “chimeric antibodies” (Morrison et al. (1984) Proc. Natl. Acad. Sci. USA 81:6851-6855; Neuberger et al. (1984) Nature 312:604-608; Takeda et al. (1985) Nature 314:452-454) by splicing the genes from a mouse antibody molecule of appropriate antigen specificity together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Similarly, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

In addition, techniques described for the production of single-chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be used. Single chain antibodies are formed by linking the heavy and light chain fragments of the Fv region via an amino acid bridge, resulting in a single-chain polypeptide.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al. (1989) Science 246:1275-1281) to allow rapid and easy identification of monoclonal Fab fragments with the desired specificity.

A large number of antibodies are commercially available. For example, monoclonal and/or polyclonal antibodies against any of a large number of specific proteins (both modified, e.g., phosphorylated, and unmodified), against phosphoserine, against phosphothreonine, against phosphotyrosine, and against any phosphoprotein (i.e., against phosphoserine, phosphothreonine and phosphotyrosine) are available, for example, from Zymed Laboratories, Inc. (www (dot) zymed (dot) com), QIAGEN, Inc. (www (dot) qiagen (dot) com) and BD Biosciences (www (dot) bd (dot) com), among many other sources. In addition, a number of companies offer services that produce antibodies against the desired antigen (e.g., a protein supplied by the customer or a peptide synthesized to order), including Abgent (www (dot) abgent (dot) com), QIAGEN, Inc. (www (dot) merlincustomservices (dot) com) and Zymed Laboratories, Inc.

### In Vivo and In Vitro Cellular Delivery

Molecules (e.g., the substrate and/or delivery modules of enzyme sensors, labeled substrates, and labeled polypeptides described herein) can be introduced into cells by traditional methods such as lipofection, electroporation, microinjection, optofection, laser transfection, calcium phosphate precipitation, cyclodextran-mediated delivery, and/or particle bombardment. Alternatively, the molecule (e.g., the substrate and/or delivery module, polypeptide, or substrate) can be associated (covalently or non-covalently) with a cellular delivery module that can mediate its introduction into the cell. The cellular delivery module is typically, but need not be, a polypeptide, for example, a PEP-1 peptide, an amphipathic peptide, e.g., an MPG peptide (Simeoni et al. (2003) “Insight into the mechanism of the peptide-based gene delivery system MPG: Implications for delivery of siRNA into mammalian cells” Nucl Acids Res 31: 2717-2724), a cationic peptide (e.g., a homopolymer of lysine, histidine, or D-arginine), or a protein transduction domain (a polypeptide that can mediate introduction of a covalently associated molecule into a cell). See, e.g., Lane (2001) Bioconju Chem., 12:825-841; Bonetta (2002) The Scientist 16:38; and Schwartz and Zhang (2000) Curr Opin Mol Ther 2:162-7. For example, a molecule can be covalently associated with a protein transduction domain (e.g., a protein transduction domain derived from an HIV-1 Tat protein, from a herpes simplex virus VP22 protein, or from a Drosophila antennapedia protein, or a model protein transduction domain, e.g., a short D-arginine homopolymer, e.g., 8-D-Arg, eight contiguous D-arginine residues). The protein transduction domain-coupled molecule can simply be, e.g., added to cell culture or injected into an animal for delivery. (Note that TAT and D-arginine homopolymers, for example, can alternatively be noncovalently associated with the molecule and still mediate its introduction into the cell.)

A number of polypeptides capable of mediating introduction of associated molecules into a cell are known in the art and can be adapted to the present invention; see, e.g., the references above and Langel (2002) Cell Penetrating Peptides CRC Press, Pharmacology & Toxicology Series.

Molecules can also be introduced into cells by covalently or noncovalently attached lipids, e.g., by lipofection or by a covalently attached myristoyl group.

The substrate and/or delivery modules, polypeptides, and substrates described herein can be introduced into a cell by any of several methods, including, without limitation, lipofection, cyclodextran, electroporation, microinjection, and covalent or noncovalent association with a cellular delivery module. Furthermore, they can optionally be introduced into specific tissues and/or cell types (e.g., explanted or in an organism), for example, by laser transfection, gold particle bombardment, microinjection, coupling to viral proteins, or covalent association with a protein transduction domain, among other techniques. See, e.g., Robbins et al. (2002) “Peptide delivery to tissues via reversibly linked protein transduction sequences” Biotechniques 33:190-192 and Rehman et al. (2003) “Protection of islets by in situ peptide-mediated transduction of the I-kappa B kinase inhibitor Nemo-binding domain peptide” J Biol Chem 278:9862-9868.

The cell into which a substrate and/or delivery module, polypeptide, or substrate of this invention is introduced can be a prokaryotic cell (e.g., a bacterial cell) or a eukaryotic cell (e.g., a yeast, a vertebrate cell, a mammalian cell, a rodent cell, a primate cell, a human cell, a plant cell, an insect cell, or essentially any other type of eukaryotic cell). The cell can be, e.g., in culture or in a tissue, fluid, etc. and/or from or in an organism.

If the molecule is caged, such delivery can be accomplished without uncaging and thereby activating the molecule; for example, a photoactivatable substrate module, polypeptide, or polypeptide substrate is not available for enzymatic modification during the delivery process until exposed to light of appropriate wavelength.

The cellular delivery modules are optionally caged. Covalently associated cellular delivery modules (e.g., protein transduction domains) can optionally be released from the associated molecule, e.g., by placement of a photolabile linkage, a disulfide or ester linkage that is reduced or cleaved in the cell, or the like, between the cellular delivery module and the molecule. For example, an 8-D-Arg module can be covalently linked through a disulfide linker to a substrate module, polypeptide, or polypeptide substrate. The 8-D-Arg module mediates entry of the substrate module, polypeptide, or polypeptide substrate into a cell, where the linker is reduced in the reducing environment of the cytoplasm, freeing the substrate module, polypeptide, or polypeptide substrate from the 8-D-Arg module.

The amount of a substrate and/or delivery module, polypeptide, or polypeptide substrate delivered to a cell can optionally be controlled by controlling the number of cellular delivery modules associated with the substrate and/or delivery module, polypeptide, or polypeptide substrate (covalently or noncovalently). For example, increasing the ratio of 8-D-Arg to substrate module, polypeptide, or polypeptide substrate can increase the percentage of substrate module, polypeptide, or polypeptide substrate that enters the cell.

The substrate and/or delivery modules, polypeptides, and substrates of this invention optionally also comprise a subcellular delivery module (e.g., a peptide, nucleic acid, and/or carbohydrate tag) or other means of achieving a desired subcellular localization (e.g., at which the enzyme is or is suspected to be present). Examples of subcellular delivery modules include nuclear localization signals, chloroplast stromal targeting sequences, and many others (see, e.g., Molecular Biology of the Cell (3rd ed.) Alberts et al., Garland Publishing, 1994; and Molecular Cell Biology (4th ed.) Lodish et al., W H Freeman & Co, 1999). Similarly, localization can be to a target protein; that is, the subcellular delivery module can comprise a binding domain that binds the target protein.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### Deep Quench: An Expanded Dynamic Range for Protein Kinase Sensors

The following sets forth a series of experiments that demonstrate synthesis and use of exemplary sensors, including exemplary kinase sensors that include a fluorescently labeled substrate module, a quencher, and a detection module.

Protein kinases catalyze the phosphorylation of serine, threonine, and tyrosine residues in protein and peptide substrates. These enzymes have received considerable attention due to the relationship between aberrant kinase activity and an assortment of human afflictions. Specific and highly sensitive protein kinase sensors furnish, e.g., a means to rapidly identify inhibitors, assess protein structure/function relationships, and correlate kinase activity with cellular behavior. A large number of kinase assays have been described; however, assays with fluorescent readouts are most easily applied to both in vitro and intracellular settings. GFP-labeled protein and fluorophore-labeled peptide substrates generally deliver, upon phosphorylation, a fluorescent response that varies from 10-60% to 2-9-fold, respectively (Rothman et al. (2005) Trends Cell Biol. 15:502-10). By comparison, many fluorescent sensors developed for a variety of biomolecules, for example, proteinases (for example, see Matayoshi et al. (1990) Science 247:954-8) and the detection of specific nucleotide sequences (Tan et al. (2004) Cur. Opin. Chem. Biol. 8:547-53), display enhancements of 25-fold and greater. A large dynamic range offers enhanced sensitivity, thereby furnishing a means to assess target biomolecule behavior under a variety of conditions. Unlike nearly all of the protein kinase assays reported to date (a few tyrosine kinase sensors that operate via an unquenching mechanism have been described: Sun et al. (2005) Anal. Chem. 77:2043-9, Wang et al. (2006) J. Amer. Chem. Soc. 128:1808-9, and Wang et al. (2006) J. Amer. Chem. Soc. 128:14016-7), the readout described in studies with proteinases (Matayoshi et al., supra) and molecular beacons (Tan et al., supra) arise via relief of fluorescent quenching. An approach devised around relief of fluorescent quenching is reported herein which delivers a robust protein kinase-elicited fluorescent response.

Initial studies focused on the strategy outlined in FIG. 1, which schematically illustrates enhanced sensing of protein kinase activity via a deeply quenched kinase peptide substrate. A fluorophore-labeled serine kinase substrate (the substrate module, with the substrate in its first state; FIG. 1 Panel A) exhibits little or no fluorescence (FIG. 1 Panel B) in the presence of a quencher molecule. Upon phosphorylation, the peptide product (the substrate module, with the substrate in its second state; FIG. 1 Panel C) is sequestered by a phospho-Ser binding domain (the detection module) to form the complex shown in FIG. 1 Panel D, which disrupts the interaction between peptide-fluorophore and quencher. The latter partially or completely restores the fluorescence of the starting peptide.

Pyrene was chosen to serve as the fluorophore on an amino acid sequence (AcGRTGRRFSYP-amide, SEQ ID NO:19) recognized by the cAMP-dependent protein kinase (“PKA”) (Mitchell et al. (1995) Biochemistry 34:528-34). The phospho-Ser binding domain, 14-3-3τ, was employed to serve as the sequestering agent since 14-3-3 domains display a high affinity for phosphoSer-containing peptides (K_{D}<100 nM) (Yaffe et al. (1997) Cell 91:961-71; a FRET-based PKA sensor has been described involving the intramolecular association of a phosphorylated amino acid sequence with 14-3-3 domain, see Zhang et al. (2001) Proc. Natl. Acad. Sci. USA 98:14997-5002). The assay was constructed in the following stages:

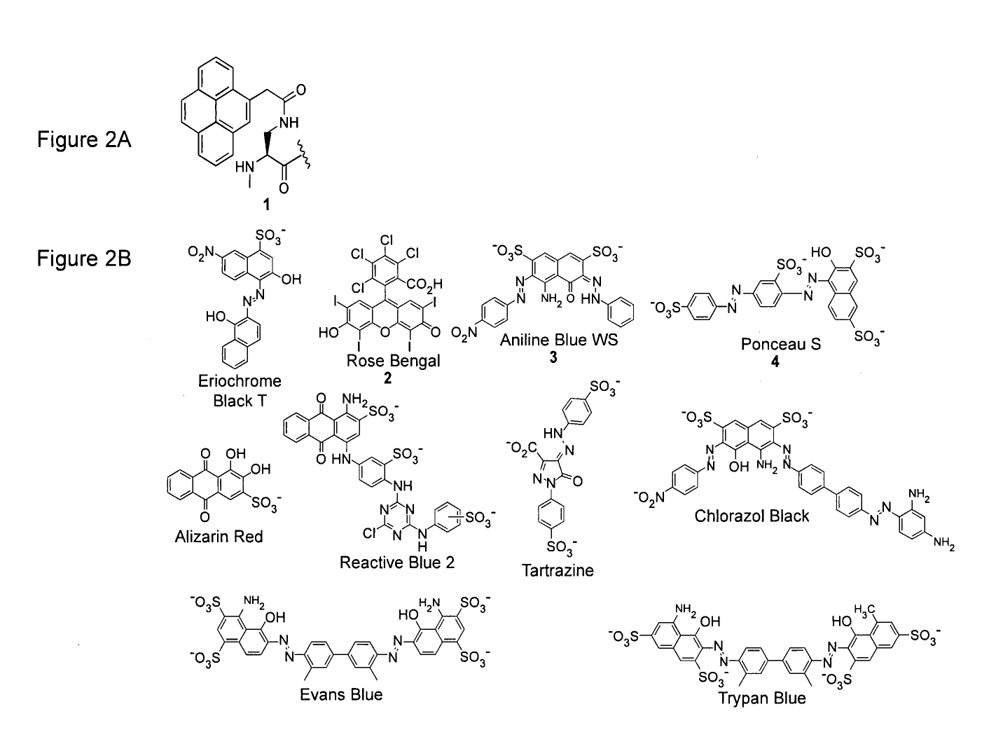

1. Identification of quenching agents. Fluorescent quenching by a secondary dye is a commonly employed method used to study a wide assortment of biological phenomena (Johansson (2006) Methods Mol. Biol. 335:17-29). However, without limitation to any particular mechanism, screening was performed for a molecule that would quench fluorophore fluorescence by forming a noncovalent complex with a targeted protein kinase peptide substrate. A library of 47 commercially available dyes (see the section entitled “Experimental Details” below) was assembled and analyzed for the ability to quench the fluorescence of a family of pyrene-substituted peptides P1-P11 (Table 1). Pyreneacetic acid (Pyr) was attached at different sites along the PKA consensus sequence peptide via a substituted 2,3-diaminopropionic (“Dap”) residue 1 (FIG. 2 Panel A) as well as to the N-terminus of the peptide via variable length linkers.

TABLE 1
Pyrene-substituted peptides P1-P11 containing
either Dap (Pyr) at the indicated internal sites
(P1-P5) or 1-pyreneactyl appended to the
1N-terminus (peptides P6-P11).
Peptide

| | | |
|---|---|---|
| P1 | Ac-GRTGRRFSDap(Pyr)P-amide | SEQ ID NO:1 |
| P2 | Ac-GRTGRRDap(Pyr)SYP-amide | SEQ ID NO:2 |
| P3 | Ac-GRTDap(Pyr)RRFSYP-amide | SEQ ID NO:3 |
| P4 | Ac-GRDap(Pyr)GRRFSYP-amide | SEQ ID NO:4 |
| P5 | Ac-Dap(Pyr)RTGRRFSYP-amide | SEQ ID NO:5 |
| P6 | Pyr-βAla-GRTGRRFSYP-amide | SEQ ID NO:6 |
| P7 | Pyr-Abu-GRTGRRFSYP-amide | SEQ ID NO:7 |
| P8 | Pyr-Ava-GRTGRRFSYP-amide | SEQ ID NO:8 |
| P9 | Pyr-Ahx-GRTGRRFSYP-amide | SEQ ID NO:9 |
| P10 | Pyr-Aoc-GRTGRRFSYP-amide | SEQ ID NO:10 |
| P11 | Pyr-Aeea-GRTGRRFSYP-amide | SEQ ID NO:11 |

Ten dyes were identified that serve as effective quenchers (>40%) of pyrene fluorescence (5 μM peptide and 5 μM dye) for several of the peptides (see the section entitled “Experimental Details” below), including Rose Bengal (2), Aniline Blue WS (3), and Ponceau S (4) (FIG. 2 Panel B). The latter, as well as the other lead quenchers, are negatively charged species. Complex formation of the quencher with the peptide is likely stabilized by electrostatic (positively charged Arg residues) and hydrophobic (fluorophore) interactions.

K_{D} values were acquired for the set of the ten lead quenchers with peptide P2, in order to obtain a target range for the quencher:peptide ratios to be employed in the subsequent assays (vide infra). These apparent K_{D} values were determined using the quenching of pyrene fluorescence as a barometer of peptide/quencher complex stability. Since the peptide/quencher pairs may interact via several different modes, not all of which might furnish efficient quenching, the actual K_{D}s could be tighter than suggested by the apparent dissociation constants. K_{D}s with peptide P2 range from 2.8±0.8 μM (Evans Blue) up to 19.6±3.4 μM (Reactive Blue) (see the section entitled “Experimental Details” below). An inner filter effect (at high dye concentrations) was corrected as previously described (Levine (1977) Clin. Chem. 23:2292-301).

2. Identification of the lead pyrene-peptide/quencher pair. The eleven pyrene-substituted peptides (P1-P11 at 5 μM) were incubated with a 5-, 10-, 25-, and 50-fold molar excess of each of the ten lead quenchers in the presence of PKA, ATP, and the phospho-Ser binding domain 14-3-3τ. Several control experiments were performed, including conducting the assay in the absence of quenching agent. Under the latter conditions, only small enhancements in fluorescence (0-64%) were observed (see the section entitled “Experimental Details” below). Since pyrene is an environmentally sensitive fluorophore, these results suggest that the phosphopeptide product binds to 14-3-3 in a manner that inserts pyrene into a modestly hydrophobic environment. At high molar excess dye ratios (>25-fold), pyrene emission is so deeply quenched that background fluorescence significantly contributes to the total fluorescence of the pyrene-peptide sample. Consequently, the background was subtracted from all readings to establish a baseline upon which changes in fluorescence intensity could be quantified (see the section entitled “Experimental Details” below).

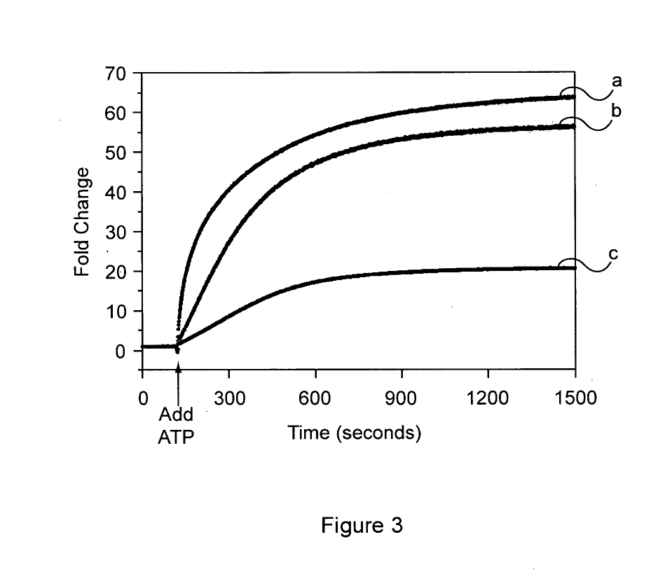

Screening, using a multiwell plate reader, revealed several unique quencher/peptide pair combinations that exhibit robust fluorescence changes in response to phosphorylation: Aniline Blue WS 3 and P9 peptide, Ponceau S 4 and P2 peptide, and Rose Bengal 2 and P5 peptide. A more detailed analysis was performed using a standard spectrofluorimeter (FIG. 3). Concentration of PKA was chosen for each pair so that the reaction would be completed within 30 min (Rose Bengal/P5, 2.5 μM); Aniline Blue WS/P9, 0.7 μM; Ponceau S/P2, 10 nM). The Rose Bengal/peptide P5 pair (FIG. 3 curve a) exhibits an unprecedented 64-fold phosphorylation-induced enhancement in fluorescence. The Aniline Blue WS/peptide P9 combination is nearly as robust (55-fold; FIG. 3 curve b), while the Ponceau S/peptide P2 pair is somewhat more subdued (21-fold; FIG. 3 curve c). The apparent K_{D}s of the two most effective pairs (Rose Bengal/peptide P5: 0.40±0.03 μM; Aniline Blue WS/peptide P9: 0.60±0.03 μM) are significantly tighter than those obtained for the ten lead dyes with peptide P2 (see the section entitled “Experimental Details” below).

The peptides P2 (Kₘ=7.1±1.9 μM; Vₘₐₓ=8.4±1.2 μmol/min-mg), P5 (Kₘ=1.7±0.4 μM; Vₘₐₓ=5.7±0.4 μmol/min-mg), and P9 (Kₘ=1.6±0.9 μM; Vₘₐₓ=7.1±1.2 μmol/min-mg) are all effective PKA substrates in the presence of 14-3-3τ. In addition, the Ponceau S/peptide P2 combination was employed to examine the inhibitory efficacy of the ATP analogue H9 (Hidaka et al. (1984) Biochemistry 23:5036-41) and a peptide fragment (14-22) of PKI (Glass et al. (1989) J. Biol. Chem. 264:8802-10), a protein-based inhibitor of PKA. Under previously reported conditions ([ATP]=10 μM), H9 is a reasonably effective inhibitor (IC₅₀=1.9±0.2 μM) of PKA. However, these conditions are nonphysiological since intracellular levels of ATP are typically above 1 mM. Under the latter conditions ([ATP]=1 mM), the potency of H9 is dramatically reduced (IC₅₀=42±1 μM), as expected for an ATP analogue. In addition, the inhibitory efficacy of the PKI 14-22 peptide inhibitor was examined under identical conditions using two different assays. Both the Deep Quench strategy described herein (1.1±0.1 μM) and the commonly employed radioactive ATP method (1.6±0.2 μM) furnish nearly identical IC₅₀ values.

In summary, a new approach for eliciting robust fluorescent readouts of protein kinase activity has been established herein.

### Experimental Details

General reagents and solvents were purchased from Fisher or Aldrich. CLEAR Rink amide resin and Fmoc-2,6-dioxoaminooctanoic acid, HCTU [1H-benzotriazolium 1-[bis(dimethylamino)methylene]-5-chloro-,hexafluorophosphate (1-),3-oxide], and HOBt-Cl (6-chloro-1-hydroxy-1H-benzotriazole) purchased from Peptides International (Louisville, Ky.). Fmoc-\tilde{β}Ala-OH, Fmoc-aminobutyric acid, Fmoc-aminovaleric acid, Fmoc-aminohexanoic acid, and Fmoc-aminooctanoic acid were purchased from Advanced Chem Tech (Louisville, Ky.). Fmoc-Dap(Mtt)-OH was purchased from Novabiochem (La Jolla, Calif.). PKA murine catalytic subunit plasmid and the GST-14-3-3τ plasmid (Aitken (2006) Semin. Cancer. Biol. 16:162-72) were generous gifts from Dr. Susan Taylor and Dr. Alistair Aitken, respectively. Dr. Hsien-ming Lee is gratefully acknowledged for a gift of PKA and Dr. Melanie Priestman for acquiring the IC₅₀ value of the PKI peptide (radioactive method).

### Synthesis of Peptide Libraries

Peptides were synthesized by standard solid phase synthesis using Fmoc chemistry. The Fmoc protecting group was removed with 20% piperidine in dimethylformamide (DMF) (1×5 min, 1×20 min). Sequential coupling of Fmoc protected amino acids was achieved with 3 equiv. Fmoc amino acid, 3 equiv. HCTU, 3 equiv. HOBt-Cl, and 6 equiv. diisopropylethylamine (DIPEA). Completion of each reaction was monitored with the Kaiser and chloranil tests. Resins were washed between steps with DMF, isopropyl alcohol (IPA), and DCM. For peptides P1-P5, the free N-terminal Gly¹ was acylated with 20 equiv. of acetic anhydride in dissolved in 1:1 pyridine:DMF. The 4-methyltrityl protecting group on Dap(Mtt) was orthogonally removed using 5% trifluoroacetic acid (TFA) and 5% triisopropylsilane (TIPS) in DCM (5 min incubation). The resulting free β-amine was acylated with 3 equiv. 1-pyreneacetic acid in DMF containing 3 equiv. HCTU, 3 equiv. HOBt-Cl, and 6 equiv. of DIPEA. The free N-termini of peptides P6-P11 were directly acylated with 1-pyreneacetic acid following the Fmoc deprotection of terminal β-alanine (βAla), aminobutyric acid (Abu), aminovaleric acid (Ava), aminohexanoic acid (Ahx), aminooctanoic acid (Aoc), and amino-3,6-dioxoaminooctanoic acid (also known as 8-amino-3,6-dioxaoctanoic acid, Aeea, and miniPEG™) groups, respectively. The remaining orthogonal protecting groups were removed and the peptides cleaved from their resins with 95% TFA, 5% water, 5% TIPS (3 hr). The peptides were isolated via filtration of the resin, precipitation with ice-cold diethyl ether, and centrifugation. The precipitates were air dried and purified by reverse-phase HPLC using a linear gradient (3%-40% acetonitrile in water with 0.1% TFA over 40 min). The peak corresponding to the desired peptide was collected, frozen, and lyophilized. The resulting white, flocculent peptides were characterized by electrospray ionization mass spectrometry: P1 Ac-Gly-Arg-Thr-Gly-Arg-Arg-Phe-Ser-Dap(Pyr)-Pro-amide (SEQ ID NO:1; m/z calculated 1403.72, found 1403.80); P2 Ac-Gly-Arg-Thr-Gly-Arg-Arg-Dap(Pyr)-Ser-Tyr-Pro-amide (SEQ ID NO:2; m/z calculated 1419.72, found 1419.60); P3 Ac-Gly-Arg-Thr-Dap(Pyr)-Arg-Arg-Phe-Ser-Tyr-Pro-amide (SEQ ID NO:3; m/z calculated 1507.75, found 1509.47); P4 Ac-Gly-Arg-Dap(Pyr)-Gly-Arg-Arg-Phe-Ser-Tyr-Pro-amide (SEQ ID NO:4; m/z calculated 1463.72, found 1464.87); P5 Ac-Dap(Pyr)-Arg-Thr-Gly-Arg-Arg-Phe-Ser-Tyr-Pro-amide (SEQ ID NO:5; m/z calculated 1507.75, found 1509.93); P6 Pyr-βAla-Gly-Arg-Thr-Gly-Arg-Arg-Phe-Ser-Tyr-Pro-amide (SEQ ID NO:6; m/z calculated 1507.75, found 1509.47); P7 Pyr-Abu-Gly-Arg-Thr-Gly-Arg-Arg-Phe-Ser-Tyr-Pro-amide (SEQ ID NO:7; m/z calculated 1521.76, found 1523.80); P8 Pyr-Ava-Gly-Arg-Thr-Gly-Arg-Arg-Phe-Ser-Tyr-Pro-amide (SEQ ID NO:8; m/z calculated 1535.78, found 1537.40); P9, Pyr-Ahx-Gly-Arg-Thr-Gly-Arg-Arg-Phe-Ser-Tyr-Pro-amide (SEQ ID NO:9; m/z calculated 1549.79, found 1551.60); P10 Pyr-Aoc-Gly-Arg-Thr-Gly-Arg-Arg-Phe-Ser-Tyr-Pro-amide (SEQ ID NO:10; m/z calculated 1577.83, found 1578.73); P11 Pyr-Aeea-Gly-Arg-Thr-Gly-Arg-Arg-Phe-Ser-Tyr-Pro-amide (SEQ ID NO:11; m/z calculated 1582.76, found 1583.73).

### Identification of Lead Quencher Dyes

The concentration of peptides P1-P11 was adjusted to 50 μM based on the molar excitation coefficient of 22,000 M⁻¹ cm⁻¹ at 345 nm. The concentrations of 47 dyes (Table 2) were adjusted to 50 μM by weight. The peptides were screened against the dyes on 96 well plates using an HTS 7000 Bio Assay Reader (Perkin Elmer) with 340 nm excitation filter and 380 nm emission filter, a setting of 100 μs integration time, and 5 flashes. Each well contained 5 μM peptide and 5 μM dye in 50 mM Tris-HCl at pH 7.5. Dyes that resulted in the greatest degree of fluorescence quenching were noted.

TABLE 2
Library of dyes.

| | |
|---|---|
| D1 | Acid Green 27 |
| D2 | Acid Blue 40 |
| D3 | Evans Blue |
| D4 | Acid Alizarin Violet N |
| D5 | Acid Blue 80 |
| D6 | Reactive Blue 2 |
| D7 | N,N-dimethylnitrosoaniline |
| D8 | Cresol Red |
| D9 | Phenol Red |
| D10 | Methyl Orange |
| D11 | Bromophenol Blue |
| D12 | BUFFER |
| D13 | Xylene Cyanol FF |
| D14 | Disperse Yellow 3 |
| D15 | Ethyl Orange |
| D16 | Methylene Blue |
| D17 | Brilliant Blue R |
| D18 | Eriochrome Black T |
| D19 | Alizarin Red |
| D20 | Malachite Green oxalate |
| D21 | Phenolphthalein |
| D22 | Carminic Acid |
| D23 | Nuclear Fast Red |
| D24 | Acid Fuchsin |
| D25 | Acridine Orange |
| D26 | Acridine Yellow G |
| D27 | Aniline Blue WS |
| D28 | Azure A |
| D29 | Azure B bromide |
| D30 | Basic Fuchsin |
| D31 | Bismark Brown Y |
| D32 | Brilliant Yellow |
| D33 | Bromocresol Purple |
| D34 | Chlorazol Black E |
| D35 | Chlorophenol Red |
| D36 | Chrysoidine Y |
| D37 | Erythrosin |
| D38 | Ethyl Violet |
| D39 | Naphthol Blue Black |
| D40 | Methylthymol Blue |
| D41 | Methyl Violet |
| D42 | Ponceau S |
| D43 | Rose Bengal |
| D44 | Rosolic Acid |
| D45 | Safranin O |
| D46 | Serva Violet 49 |
| D47 | Tartrazine |
| D48 | Trypan Blue |

Acquisition of Apparent K_{D} Values for Lead Quencher Dyes with Peptide P2

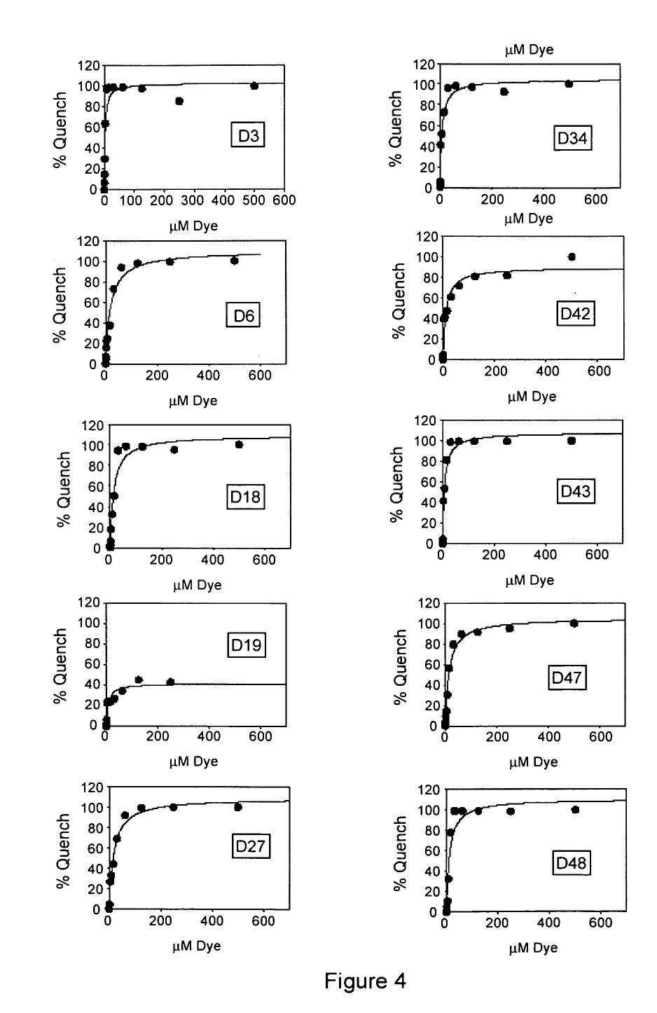

Varying concentrations of 10 dyes (FIG. 2 Panel B), ranging from 0.5-500 μM, were added to 5 μM pyrene-labeled P2 peptide in 100 mM Tris HCl pH 7.5 buffer (96 well plates). A Spectra Max Gemini EM plate reader (Molecular Devices) was used for fluorescence measurements (λₑₓ=342 nm and (λₑₘ=380 nm); see FIG. 4. Correction for the inner filter effect was made using the antilogarithm of the effective optical density times half the width of the fluorescence well as previously reported (Clin. Chem 23 (12) 2292-2301, 1977). Molar absorbtivities (ε₃₄₂ and ε₃₈₀) were calculated from single absorbance spectra at a [dye]=7.81 μM. For all dyes at concentrations below 10 μM, the inner filter effect required a correction of less than 10% in the measured fluorescence. However, at higher concentrations, the effect became significant for strongly absorbing dyes. After correcting for the inner filter effect, the percentage of quench was plotted against the concentration of the dye. A nonlinear regression analysis fit of the data to the rectangular hyperbola model using the Sigma Plot version 8.02 software was used to obtain apparent K_{D} values. Apparent K_{D} values are presented in Table 3.

TABLE 3
Apparent K_{D} values of lead quenchers with peptide P2.

| Quencher Dye | | Apparent K_{D} (μM) |
|---|---|---|
| D3 | Evans Blue | 2.8 ± 0.8 |
| D6 | Reactive Blue 2 | 19.6 ± 3.4 |
| D18 | Eriochrome Black | 14.3 ± 3.3 |
| D19 | Alizarin Red | 7.3 ± 2.5 |
| D27 | Aniline Blue WS | 18.1 ± 2.6 |
| D34 | Chlorazol Black E | 7.7 ± 1.5 |
| D42 | Ponceau S | 11.2 ± 2.7 |
| D43 | Rose Bengal | 7.5 ± 1.6 |
| D47 | Tartrazine | 15.0 ± 2.1 |
| D48 | Trypan Blue | 11.9 ± 3.6 |

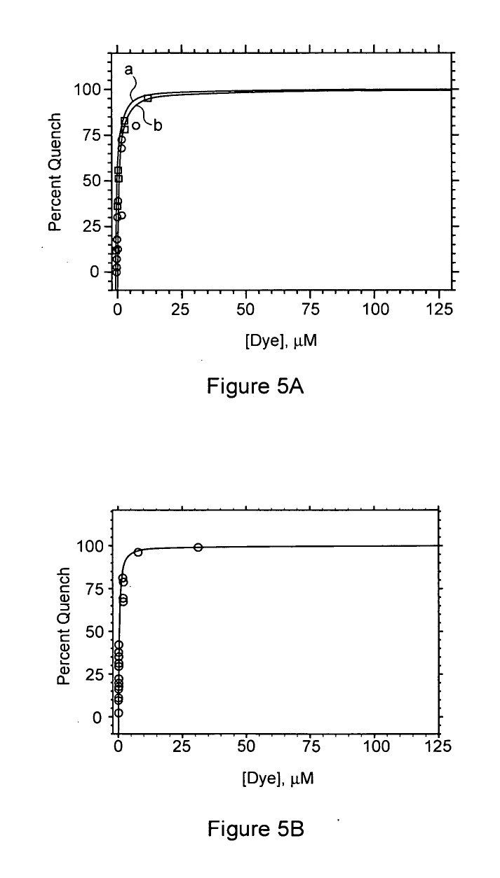

Acquisition of apparent K_{D} values for lead quencher/peptide pairs were performed as described above and, as noted previously, are 0.40±0.03 μM for Rose Bengal/peptide P5 and 0.60±0.03 μM for Aniline Blue WS/peptide P9. See FIG. 5 Panel A. In addition, the apparent K_{D} value for the phosphorylated P5 peptide AcDap(Pyr)RTGRRFS(PO₃²⁻)YP-amide (SEQ ID NO:20) with Rose Bengal is 210±40 nM (see FIG. 5 Panel B), slightly tighter than that found for the unphosphorylated P5 peptide AcDap(Pyr)RTGRRFSYP-amide (SEQ ID NO:5)/Rose Bengal pair (400±30 nM).

Screening of Lead Quenching Dyes 1-10 with Peptides P1-P11

PKA-catalyzed phosphorylation was initiated by addition of 25 μL of 100 nM PKA enzyme to the following solution: 25 μL 50 μM fluorescent peptide substrates (P1-P11), 25 μL 20 mM DTT, 25 μL 10 mM ATP, 25 μL 50 mM MgCl₂, 25 μL 100 μM 14-3-3%, 25 μL 0.5 M Tris HCl pH 7.5, 25 μL dye (10 dyes at 4 concentrations, 0.25 mM, 0.5 mM, 1.25 mM 2.5 mM and no dye as a control) to give final volume of 250 μL. The concentrations per well were: 10 nM PKA, 5 μM peptide, 10 μM 14-3-3%, 1 mM ATP, 5 mM MgCl₂, 2 mM DTT, and 0 μM (control, see Table 4), 25 μM, 50 μM, 125 μM or 250 μM each of 10 different lead dyes in 50 mM Tris at pH 7.5 buffer (see Table 6). For the 0 μM control, a Photon Technology QM-1 spectrofluorimeter was set in time-based mode with an 8 nm slit-width at 30° C. and 343 nm as the excitation wavelength and 380 nm emission wavelength. For the assays with varying dye concentrations, a Molecular Devices Spectra Max EM plate reader was set in kinetic mode to read from the bottom of a 96 well Costar 3631 flat bottom 96 at 30° C. using 343 nm excitation wavelength and 380 nm emission wavelength.

TABLE 4
Control experiment: Phosphorylation-induced change
in fluorescence of pyrene-labeled peptides P1-P11
in the absence of quenching dye.

| | |
|---|---|
| | % Fluorescence |
| Pyrene-labeled Peptide | Enhancement |

| | | |
|---|---|---|
| P1 | Ac-GRTGRRFSDap(Pyr)P-amide; | 0 |
| | SEQ ID NO:1 | |
| P2 | Ac-GRTGRRDap(Pyr)SYP-amide; | 51% |
| | SEQ ID NO:2 | |
| P3 | Ac-GRTDap(Pyr)RRFSYP-amide; | 19% |
| | SEQ ID NO:3 | |
| P4 | Ac-GRDap(Pyr)GRRFSYP-amide; | 40% |
| | SEQ ID NO:4 | |
| P5 | Ac-Dap(Pyr)RTGRRFSYP-amide; | 64% |
| | SEQ ID NO:5 | |
| P6 | Pyr-βAla-GRTGRRFSYP-amide; | 49% |
| | SEQ ID NO:6 | |
| P7 | Pyr-Abu-GRTGRRFSYP-amide; | 47% |
| | SEQ ID NO:7 | |
| P8 | Pyr-Ava-GRTGRRFSYP-amide; | 31% |
| | SEQ ID NO:8 | |
| P9 | Pyr-Ahx-GRTGRRFSYP-amide; | 48% |
| | SEQ ID NO:9 | |
| P10 | Pyr-Aoc-GRTGRRFSYP-amide; | 39% |
| | SEQ ID NO:10 | |
| P11 | Pyr-Aeea-GRTGRRFSYP-amide; | 38% |
| | SEQ ID NO:11 | |

### Beer's Law Analysis

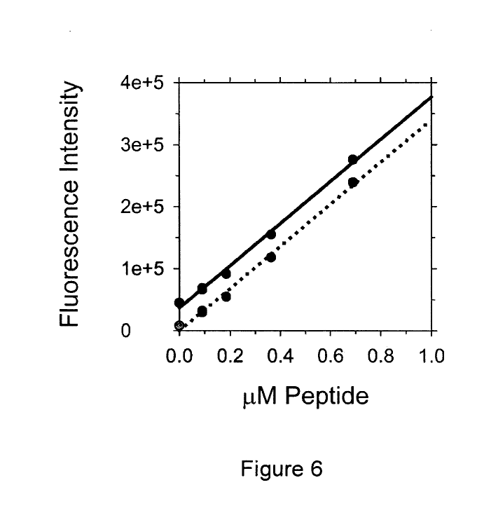

The fluorescence intensities of different concentrations of phosphorylated P5 peptide (ranging from 0 to 1 μM and incubated with 10 μM 14-3-3τ and 100 mM Tris HCl pH 7.5) were determined in the presence of 12.5 μM Rose Bengal. The intensities were plotted against the peptide concentration and the data fit to a straight line (FIG. 6, solid line). The fit of the data with background correction is shown as a dotted line in FIG. 6. The background was acquired by using a sample that had all the assay components except the fluorophore-peptide by using the “Acquire Background” mode in FeliX software (Photon Technology version 1.42). This background intensity was automatically subtracted from subsequent measurements by the software.

### Acquisition of Kₘ and Vₘₐₓ Values

Phosphorylation dependent increase in pyrene fluorescence intensity of peptides P2, P5 and P9 were monitored on a Photon Technology QM-1 spectrofluorimeter at 30° C. using 343 nm excitation wavelength, 380 nm emission wavelength, and an 8 nm slit-width. After equilibration of different concentrations of the pyrene-labeled peptide substrate with 50 mM Tris buffer pH 7.5, 30 μM 14-3-3τ, 1 mM ATP, 5 mM MgCl₂, 2 mM DTT, for 10 min, 10 nM enzyme was added and the reaction progress curves obtained. Reaction rates were determined from the slope under conditions where 5-8% substrate had been converted to product in duplicate. The resulting slopes (initial velocity, v₀) for each of the progress curves were plotted versus the concentration of substrate. A nonlinear regression analysis was used to fit the data to the rectangular hyperbola model using the Sigma Plot version 8.02 software.

### Assay Dependence on 14-3-3τ

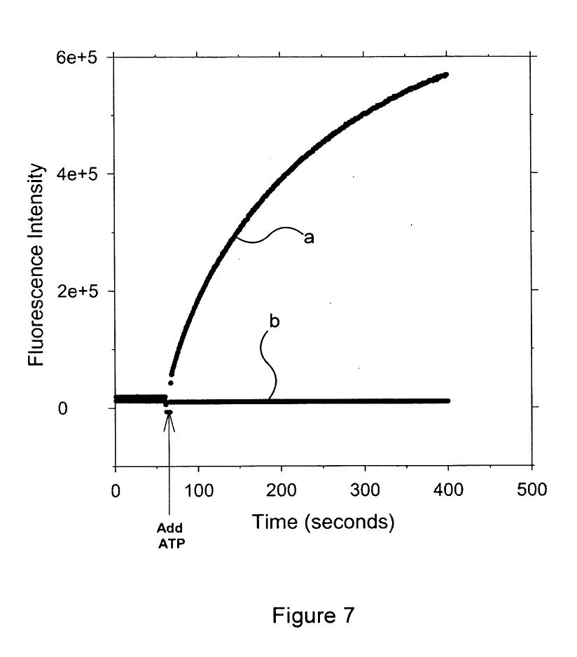

Phosphorylation-dependent increase in pyrene fluorescence intensity of peptide P5, in the presence and absence of 14-3-3%, was monitored on a Photon Technology QM-1 spectrofluorimeter at 30° C. using 343 nm excitation wavelength, 380 nm emission wavelength with an 8 nm slit-width. 5 μM pyrene-labeled peptide substrate P5 was pre-incubated in 25 μM Rose Bengal, 5 mM MgCl₂, 2 mM DTT, 1.4 μM PKA, and 50 mM Tris buffer pH 7.5, in the presence (FIG. 7 curve a), and absence (FIG. 7 curve b), of 30 μM 14-3-3τ, at 30° C. for 5 min. After 1 min, 1 mM ATP was added and the reaction progress followed. In the absence of 14-3-3% (FIG. 7 curve b), no change in fluorescence intensity was observed.

### Inhibitor IC₅₀ Values

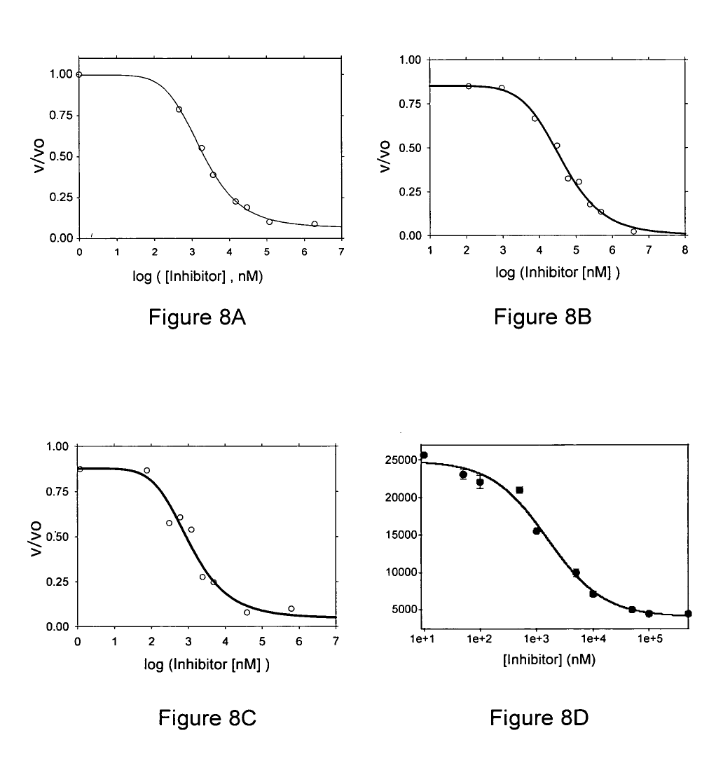

1 μM pyrene-labeled peptide substrate P2 was incubated in 60 μM Ponceau S, 50 mM Tris buffer pH 7.5, 1 mM ATP, 30 μM 14-3-3τ, 5 mM MgCl₂, 2 mM DTT at 30° C. for 5 min. 10 nM PKA enzyme was added and the reaction progress followed for 1 min. This step was used to adjust for inter-assay variability and to verify that no significant drop in enzyme activity occurs over the course of the determinations. Subsequently, inhibitor was added at different concentrations. Reaction rates were measured under conditions where less than 10% substrate had been converted to product. Fractional velocities (v/v₀) were plotted against inhibitor concentration [I] (FIG. 8 Panels A-D) and fit using the Sigma Plot version 8.02 software's four-parameter logistic nonlinear regression analysis. PKI (14-22) exhibits an IC₅₀ value of 1.1±0.1 μM. The IC₅₀ value of PKI (14-22) using the standard radioactive ATP method is 1.6±0.2 μM. H9.HCl exhibits an IC₅₀ value of 42±1 μM at 1 mM ATP and a value of 1.9±0.2 μM at 10 μM ATP.

### Fluorescence Change Dependency on Instrumentation/Reading Mode

The phosphorylation-induced fluorescence change was found to be dependent upon instrumentation and reading mode. In brief, the least dramatic changes are observed in a plate reader (Molecular Devices Spectra Max Gemini EM) using the bottom read mode (i.e. from below through the bottom of the clear well plates: Costar 3631 flat bottom 96 multiwell plates). A more robust change is obtained via a top read mode (Molecular Devices Spectra Max Gemini) for a multiwell plate (Wallac B & W isoplate 1450-582). The highest fluorescence fold change is provided using a dedicated spectrofluorimeter (Photon Technology QM-1) and a quartz cuvette as the sample holder. These results are summarized in Table 5 for the three lead peptide/quencher pairs. Initial screening of the library of peptides P1-P11 with the ten lead quenchers (FIG. 2 Panel B) was performed using the bottom read mode. A summary of these results is furnished in Table 6.

An example of assay conditions in plate reader mode is furnished for the top read with peptide P5 and Rose Bengal: Phosphorylation-dependent increase in pyrene fluorescence intensity of peptide dye pair P5/Rose Bengal, was monitored on a Molecular Devices Spectra Max Gemini plate reader at 30° C. using 330 nm excitation wavelength, 380 nm emission wavelength. Three wells containing 5 μM pyrene-labeled peptide substrate P5 were pre-incubated in 5 mM MgCl₂, 2 mM DTT, 1 mM ATP, and 50 mM Tris buffer pH 7.5, 30 μM 14-3-3τ and 25 μM Rose Bengal, at 30° C. for 10 min. 0.7 μM PKA was added and the reaction progress followed. Three additional wells containing all the assay components except for P5 peptide were used for blank readings.

TABLE 5
Phosphorylation-induced fluorescence fold-change of lead
peptide/quencher pairs as a function of instrumentation/read mode.

| Peptide/Quencher | | Fluorescence |
|---|---|---|
| (ratio) | Conditions | Change |
| P2/Ponceau S (1:50) | Plate reader - bottom read | 7-fold |
| | Plate reader - top read | 15-fold |
| | spectrofluorimeter | 21-fold |
| P5/Rose Bengal (1:5) | Plate reader - bottom read | 8-fold |
| | Plate reader - top read | 30-fold |
| | spectrofluorimeter | 64-fold |
| P9/Aniline Blue WS (1:10) | Plate reader - bottom read | 9-fold |
| | Plate reader - top read | 19-fold |
| | spectrofluorimeter | 55-fold |

TABLE 6
PKA-induced fluorescence change of peptide P1-P11 in the presence of the ten lead quencher
dyes. Peptide concentration was fixed at 5 μM and a 5-, 10-, 25-, and 50-fold excess of
quencher was employed.

| | Fold | | | | | | | | | | | |
|---|---|---|---|---|---|---|---|---|---|---|---|---|
| | excess | Peptide | | | | | | | | | | |
| Dye | dye | P1 | P2 | P3 | P4 | P5 | P6 | P7 | P8 | P9 | P10 | P11 |
| D3 | 50 | 1.15 | 0.94 | 0.99 | 0.91 | 0.78 | 0.70 | 0.81 | 0.88 | 0.83 | 0.73 | 0.74 |
| Evans | 25 | 1.46 | 1.01 | 1.06 | 0.89 | 0.84 | 0.06 | 0.45 | 0.89 | 0.26 | 0.59 | 0.25 |
| Blue | 10 | 1.43 | 1.37 | 1.06 | 0.91 | 0.92 | 0.74 | 0.31 | 0.36 | 0.24 | 0.64 | 0.12 |
| | 5 | 1.69 | 1.92 | 1.10 | 0.98 | 1.33 | 1.50 | 1.01 | 1.34 | 1.09 | 0.69 | 0.46 |
| D6 | 50 | 3.85 | 6.30 | 1.68 | 4.27 | 5.79 | 4.70 | 5.20 | 5.01 | 4.03 | 3.48 | 2.46 |
| Reactive | 25 | 2.92 | 5.66 | 3.56 | 3.55 | 6.44 | 4.78 | 4.43 | 5.33 | 4.95 | 3.95 | 3.09 |
| Blue 2 | 10 | 1.57 | 2.08 | 2.29 | 2.66 | 3.99 | 2.33 | 3.68 | 3.40 | 3.61 | 3.80 | 1.72 |
| | 5 | 1.00 | 1.99 | 1.48 | 2.16 | 2.28 | 1.63 | 2.64 | 2.57 | 3.39 | 5.12 | 1.57 |
| D18 | 50 | 1.10 | 3.71 | 0.31 | 0.59 | 1.12 | 1.52 | 0.48 | 0.45 | 0.65 | 0.72 | 0.30 |
| Eriochrome | 25 | 0.41 | 1.13 | 0.58 | 0.59 | 0.84 | 0.87 | 1.58 | 0.62 | 1.46 | 0.79 | 0.56 |
| Black T | 10 | 1.13 | 1.42 | 1.02 | 1.12 | 1.73 | 1.37 | 1.64 | 1.47 | 1.52 | 2.23 | 0.71 |
| | 5 | 0.96 | 1.46 | 1.10 | 1.32 | 1.61 | 1.37 | 1.82 | 1.65 | 2.09 | 2.51 | 1.42 |
| D19 | 50 | 1.13 | 1.30 | 1.05 | 1.07 | 1.50 | 1.27 | 1.34 | 1.64 | 2.03 | 2.44 | 1.14 |
| Alizarin | 25 | 1.01 | 1.37 | 1.17 | 1.23 | 1.49 | 1.32 | 1.60 | 1.59 | 1.91 | 1.95 | 1.28 |
| Red | 10 | 0.96 | 1.38 | 1.21 | 1.33 | 1.59 | 1.27 | 1.63 | 1.57 | 1.98 | 1.90 | 1.35 |
| | 5 | 0.98 | 1.59 | 1.28 | 1.36 | 1.70 | 1.35 | 1.71 | 1.68 | 2.01 | 1.97 | 1.31 |
| D27 | 50 | 1.55 | 1.83 | 0.99 | 1.40 | 1.23 | 1.28 | 1.39 | 1.35 | 1.28 | 1.09 | 1.63 |
| Aniline | 25 | 4.13 | 2.52 | 3.79 | 5.58 | 6.04 | 5.60 | 4.76 | 1.81 | 3.59 | 2.39 | 2.21 |
| Blue | 10 | 0.47 | 2.48 | 2.17 | 4.82 | 5.36 | 3.48 | 6.63 | 6.22 | 8.94 | 6.77 | 1.78 |
| WS | 5 | 1.16 | 1.85 | 1.50 | 2.34 | 3.02 | 2.35 | 3.85 | 4.07 | 6.22 | 6.82 | 1.62 |
| D34 | 50 | 0.10 | 0.35 | 0.25 | 0.87 | 0.25 | 0.38 | 0.95 | 0.97 | 0.85 | 0.89 | 0.19 |
| Chlorazol | 25 | 0.49 | 0.60 | 0.92 | 0.55 | 0.21 | 1.10 | 1.08 | 1.05 | 0.88 | 0.74 | 0.86 |
| Black E | 10 | 2.44 | 2.84 | 0.71 | 0.93 | 1.52 | 2.32 | 2.49 | 2.83 | 1.61 | 1.32 | 0.83 |
| | 5 | 1.42 | 2.26 | 1.79 | 4.01 | 5.62 | 2.27 | 4.81 | 6.65 | 3.63 | 4.80 | 1.62 |
| D42 | 50 | 3.37 | 6.57 | 2.88 | 2.69 | 4.36 | 1.77 | 1.27 | 3.26 | 4.02 | 2.81 | 2.38 |
| Ponceau | 25 | 1.84 | 4.61 | 2.75 | 2.88 | 3.23 | 3.13 | 2.19 | 2.01 | 4.64 | 5.66 | 2.12 |
| S | 10 | 1.76 | 1.92 | 2.26 | 2.63 | 3.30 | 3.36 | 1.92 | 3.86 | 4.83 | 7.99 | 2.06 |
| | 5 | 1.35 | 1.93 | 1.81 | 2.26 | 2.85 | 2.77 | 2.00 | 3.14 | 4.32 | 7.56 | 2.10 |
| D43 | 50 | 0.96 | 0.72 | 0.97 | 0.91 | 0.95 | 0.94 | 0.85 | 0.95 | 0.98 | 0.89 | 0.84 |
| Rose | 25 | 1.03 | 1.58 | 1.13 | 1.10 | 1.02 | 1.20 | 0.85 | 0.81 | 1.11 | 1.23 | 0.32 |
| Bengal | 10 | 2.63 | 2.87 | 1.98 | 2.88 | 3.45 | 3.28 | 1.12 | 2.19 | 1.85 | 1.32 | 0.94 |
| (Cert) | 5 | 1.20 | 2.58 | 2.70 | 6.52 | 8.36 | 2.71 | 7.44 | 5.43 | 3.51 | 3.26 | 3.54 |
| D47 | 50 | 1.31 | 2.51 | 1.02 | 1.47 | 2.05 | 1.25 | 1.65 | 1.79 | 1.53 | 1.92 | 1.43 |
| Tartrazine | 25 | 1.16 | 1.82 | 1.47 | 1.46 | 1.79 | 1.42 | 1.77 | 1.58 | 1.82 | 1.76 | 1.28 |
| | 10 | 1.06 | 1.61 | 1.34 | 1.42 | 1.52 | 1.27 | 1.55 | 1.47 | 1.57 | 1.64 | 1.17 |
| | 5 | 0.99 | 1.41 | 1.19 | 1.28 | 1.43 | 1.23 | 1.46 | 1.39 | 1.55 | 1.47 | 1.15 |
| D48 | 50 | 2.60 | 6.03 | 1.60 | 2.76 | 3.48 | 3.34 | 5.62 | 3.52 | 2.62 | 3.39 | 3.17 |
| Trypan | 25 | 3.40 | 5.11 | 1.91 | 1.72 | 3.61 | 3.05 | 4.10 | 4.03 | 3.67 | 3.53 | 2.45 |
| Blue | 10 | 3.04 | 2.81 | 2.72 | 2.18 | 2.72 | 2.17 | 4.76 | 4.87 | 3.96 | 3.45 | 3.40 |
| | 5 | 1.30 | 2.20 | 2.23 | 3.03 | 6.21 | 3.54 | 7.15 | 6.14 | 7.18 | 7.22 | 3.06 |

### Additional Exemplary Sensors

Coumarin (Cou) labeled-peptide P12 (Cou-Aoc-GRTGRRFSYP-amide (SEQ ID NO:12), shown in FIG. 9 Panel A), was also prepared. P12 contains the coumarin derivative diethylaminocoumarin (λ_{excitation}=425 nm and λₑₘᵢₛₛᵢₒₙ=470 nm); 7-diethylaminocoumarin-3-carboxylic acid (catalog number D-1421 from Invitrogen) was coupled to the free amine of the peptide. Using P12 as the substrate module with an Acid Green 27 quencher and a 14-3-3 detection module, a 225-fold enhancement in fluorescence was observed (see FIG. 9 Panel B). Concentrations in the assay were 1.6 μM peptide P12, 10 μM 14-3-3, and 30 μM Acid green 27.

It will be evident that a variety of sensors related to those described herein are contemplated and are included in the scope of the appended claims. For example, a number of sensors related to P1-P12 can be prepared by employing different fluorescent labels. As just a few examples, such additional sensors include, but are not limited to, Ac-GRTGRRDap(Coumarin)SYP-amide (SEQ ID NO:31), Ac-GRTGRRDap(TAMRA)SYP-amide (SEQ ID NO:32), Ac-Dap(Coumarin)RTGRRFSYP-amide (SEQ ID NO:33), and Ac-Dap(TAMRA)RTGRRFSYP-amide (SEQ ID NO:34).

While the preceding examples have focused on serine kinase sensors, as noted above, the strategy outlined herein is applicable to other enzymes as well, for example: 1) tyrosine protein kinases, using, e.g., SH2 or PTB domains to capture the phosphorylated product, 2) methyltransferases, e.g., histone transferases with respect to epigenomics, using, e.g., a Chromo domain to capture the methylated lysine peptide, and 3) acetyltransferases, e.g., histone acetyltransferases with respect to epigenomics, using, e.g., a Bromo domain to capture the acetylated lysine peptide.

1) Tyrosine protein kinases. An exemplary tyrosine kinase sensor is schematically illustrated in FIG. 10. A substrate module that includes a fluorophore-labeled tyrosine kinase substrate in its first, unphosphorylated state is shown in Panel A. As shown in Panel B, the fluorophore exhibits little or no fluorescence in the presence of a quencher. Upon phosphorylation of the substrate (i.e., conversion of the substrate to the second, phosphorylated state by a kinase; Panel C), the detection module (an SH2 domain in this example) binds to the substrate module, disrupting the interaction between the fluorophore and the quencher and thus resulting in increased fluorescent emission from the fluorophore. Typically, binding of the detection module partially or completely restores the fluorescence of the starting fluorophore-labeled substrate.

2) Methyltransferases. An exemplary methyltransferase sensor is schematically illustrated in FIG. 11. A substrate module that includes a fluorophore-labeled substrate in its first, unmethylated state is shown in Panel A. As shown in Panel B, the fluorophore exhibits little or no fluorescence in the presence of a quencher. Upon methylation of the substrate (i.e., conversion of the substrate to the second, methylated state by a methyltransferase; Panel C), the detection module (a chromo domain in this example) binds to the substrate module, disrupting the interaction between the fluorophore and the quencher and thus resulting in increased fluorescent emission from the fluorophore.

3) Acetyltransferases. An exemplary acetyltransferase sensor is schematically illustrated in FIG. 12. A substrate module that includes a fluorophore-labeled substrate in its first, unacetylated state is shown in Panel A. As shown in Panel B, the fluorophore exhibits little or no fluorescence in the presence of a quencher. Upon acetylation of the substrate (i.e., conversion of the substrate to the second, acetylated state by an acetyltransferase; Panel C), the detection module (a bromo domain in this example) binds to the substrate module, disrupting the interaction between the fluorophore and the quencher and thus resulting in increased fluorescent emission from the fluorophore.

Also as noted above, a general strategy is presented for any peptide or protein biosensor that binds to a target protein. An exemplary binding sensor is schematically illustrated in FIG. 13. A labeled polypeptide that includes a fluorophore-labeled first polypeptide is shown in Panel A (the sensor peptide or protein). As shown in Panel B, the fluorophore exhibits little or no fluorescence in the presence of a quencher. Binding of the first polypeptide to a second polypeptide (the target protein) at least partially sequesters the fluorophore from the quencher, disrupting the interaction between the fluorophore and the quencher and thus resulting in increased fluorescent emission from the fluorophore as shown in Panel C. Typically, binding of the second polypeptide partially or completely restores the fluorescence of the starting fluorophore-labeled sensor polypeptide. Such binding sensors can be used to detect and/or quantitate intermolecular association between polypeptides.

For example, interaction between a fluorophore-labeled proline rich peptide and an SH3 domain can be detected as schematically illustrated in FIG. 14. A fluorophore-labeled proline rich first polypeptide is shown in Panel A. As shown in Panel B, the fluorophore exhibits little or no fluorescence in the presence of a quencher. As shown in Panel C, binding of the proline rich polypeptide to the SH3 domain disrupts the interaction between the fluorophore and the quencher, resulting in increased fluorescent emission from the fluorophore.

### Example 2

### Enzyme Sensors with Covalently Attached Quenchers

The following sets forth a series of experiments that demonstrate synthesis and use of exemplary sensors. The exemplary sensors include kinase sensors that have a substrate module with a fluorophore and quencher and a detection module, as well as kinase sensors that have a substrate with a fluorophore and quencher and that do not require detection modules.

Pyrene-substituted peptides P13 (FIG. 15 Panel A, SEQ ID NO:21) and P14 (FIG. 15 Panel B, SEQ ID NO:22) were synthesized. Upon phosphorylation by PKA, P13 displays a 1.5-2.3 fold increase in fluorescence in the presence of 14-3-3%, and P14 displays a 2.2 fold increase in the presence of 14-3-3%. No significant change in fluorescence is observed in the absence of 14-3-3%. The assay was performed with a total volume of 200 μL and was initiated with the addition of PKA enzyme. The final concentrations of the reaction components are: 1 mM ATP, 1.5 mM MgCl₂, 2.1 μM 14-3-3τ, 2.6 μM P13 substrate (or 6.3 μM for P14 substrate), and 24 nM PKA.

A panel of peptides including PKA substrate GRTGRRX^{X-1}SLPK⁺³ (SEQ ID NO:24) with a pyrene-substituted Dap residue at position X⁻¹ was synthesized: peptides P15-P20 (Table 7). P15-P20 have a polyGlu peptide bearing the negatively charged quencher Reactive Blue 2 attached to the Nε of K⁺³ (the Nε is acylated by the alpha-carboxyl of the C-terminal Glu or Dap residue of the peptide). FIG. 16 schematically illustrates operation of this class of sensors.

TABLE
Reactive Blue 2-pyrene-polyGlu peptides.

| | | |
|---|---|---|
| P15 | 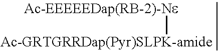 | SEQ ID NO:25 |
| P16 | 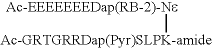 | SEQ ID NO:26 |
| P17 | 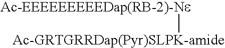 | SEQ ID NO:27 |
| P18 | 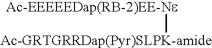 | SEQ ID NO:28 |
| P19 | 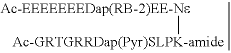 | SEQ ID NO:29 |
| P20 | 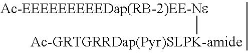 | SEQ ID NO:30 |

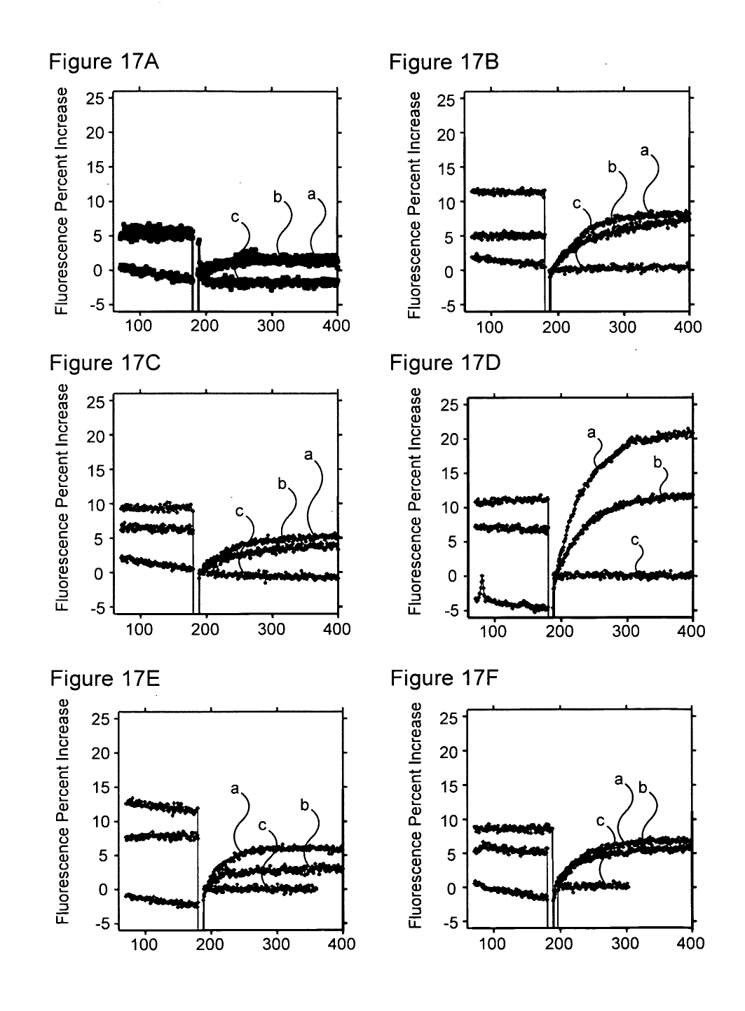

Performance of these sensors upon phosphorylation by PKA in the presence or absence of a 14-3-3 detection module was assessed (FIG. 17 Panels A-F). 211M of each peptide (P15-P20, FIG. 17 Panels A-F, respectively) was incubated at 30° C. with 1 mM ATP, 5 mM MgCl₂, 2 mM DTT, 50 mM Tris HCl pH 7.5 either with 5 μM 14-3-3τ (curve a) or without 14-3-3τ (curves b and c). After three minutes, either 1 μL of buffer (curve c) or 1 μL of 5 μM PKA enzyme (curves a and b; concentration of enzyme in assay was 20 nM) was added to the sample and the change in fluorescence monitored. Results are summarized in Table 8.

TABLE 8
Fluorescence percent increase upon phosphorylation.

Percent Increase

| peptide | without 14-3-3 | with 14-3-3 |
|---|---|---|
| P15 | 2.1 | 1.5 |
| P16 | 7.2 | 8.1 |
| P17 | 3.4 | 5.4 |
| P18 | 12.0 | 20.8 |
| P19 | 3.3 | 5.9 |
| P20 | 5.8 | 6.8 |

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A composition comprising: a sensor for detecting an activity of a enzyme, the sensor comprising
a substrate for the enzyme, wherein the substrate is in a first state on which the enzyme can act, thereby converting the substrate to a second state,
a fluorescent label covalently connected to the substrate, and
a quencher covalently connected to the substrate,
wherein when the substrate is in the first state florescent emission by the label is quenched by the quencher,
wherein conversion of the substrate from the first state to the second state alters the net charge of the substrate, introducing an unfavorable intramolecular electrostatic interaction or eliminating a favorable intramolecular electrostatic interaction and thereby resulting in a conformational change in the sensor that at least partially relieves quenching of the label by the quencher.

2-25. (canceled)

26. A method of assaying an activity of an enzyme, the method comprising:
a) contacting the enzyme with a sensor, the sensor comprising
i) a substrate for the enzyme, wherein the substrate is in a first state on which the enzyme can act, thereby converting the substrate to a second state,
ii) a fluorescent label covalently connected to the substrate, and
iii) a quencher covalently connected to the substrate,
wherein when the substrate is in the first state florescent emission by the label is quenched by the quencher,
wherein conversion of the substrate from the first state to the second state alters the net charge of the substrate, introducing an unfavorable intramolecular electrostatic interaction or eliminating a favorable intramolecular electrostatic interaction and thereby resulting in a conformational change in the sensor that at least partially relieves quenching of the label by the quencher and results in an increased intensity of fluorescent emission from the label;
b) detecting the increased intensity of fluorescent emission from the label; and
c) correlating the increased intensity of fluorescent emission from the label to the activity of the enzyme, thereby assaying the activity of the enzyme.

27. The method of claim 26, wherein the substrate is a polypeptide substrate.

28. The method of claim 27, wherein conversion of the substrate from the first state to the second state alters the charge of an amino acid side chain.

29. The method of claim 28, wherein conversion of the substrate from the first state to the second state involves transfer of a functional group to the side chain.

30. The method of claim 28, wherein conversion of the substrate from the first state to the second state involves removal of a functional group from the side chain.

31. The method of claim 28, wherein the amino acid side chain in the first state is uncharged and in the second state is negatively charged.

32. The method of claim 31, wherein the quencher is negatively charged, and wherein conversion of the substrate from the first state in which the amino acid side chain is uncharged to the second state in which the side chain is negatively charged introduces an unfavorable electrostatic interaction between the quencher and the side chain.

33. The method of claim 31, wherein one or more amino acid residues adjacent to the quencher are negatively charged, and wherein conversion of the substrate from the first state in which the amino acid side chain is uncharged to the second state in which the side chain is negatively charged introduces an unfavorable electrostatic interaction between the side chain and the residues.

34. The method of claim 31, wherein the amino acid side chain is a serine, threonine, or tyrosine side chain which is unphosphorylated in the first state and phosphorylated in the second state.

35. The method of claim 31, wherein the polypeptide substrate comprises the amino acid sequence of SEQ ID NO:24.

36. The method of claim 31, wherein the sensor is selected from the group consisting of:

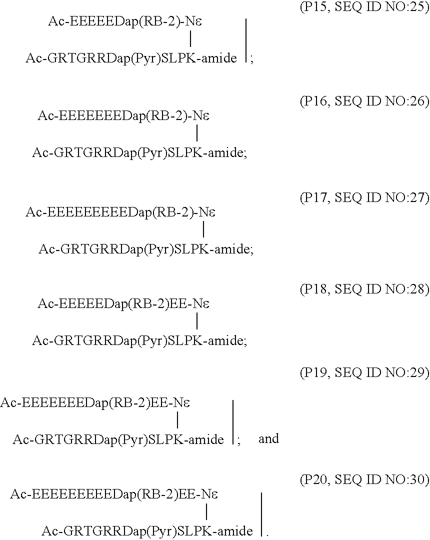

37. The method of claim 28, wherein the amino acid side chain in the first state is positively charged and in the second state is uncharged.

38. The method of claim 37, wherein the quencher is negatively charged, and wherein conversion of the substrate from the first state in which the amino acid side chain is positively charged to the second state in which the side chain is uncharged eliminates a favorable electrostatic interaction between the quencher and the side chain.

39. The method of claim 37, wherein one or more amino acid residues adjacent to the quencher are negatively charged, and wherein conversion of the substrate from the first state in which the amino acid side chain is positively charged to the second state in which the side chain is uncharged eliminates a favorable electrostatic interaction between the side chain and the residues.

40. The method of claim 37, wherein the amino acid side chain is an arginine or lysine side chain which is unmethylated in the first state and methylated in the second state, or wherein the amino acid side chain is a lysine side chain which is unacetylated in the first state and acetylated in the second state.

41. The method of claim 28, wherein the fluorescent label is adjacent to the residue whose side chain is modified.

42. The method of claim 27, wherein the enzyme is selected from the group consisting of: a protein kinase, a serine/threonine protein kinase, a tyrosine protein kinase, a histone methyltransferase, a histone lysine methyltransferase, a histone arginine methyltransferase, a protein lysine methyltransferase, a histone acetyltransferase, a lysine acetyltransferase, and a protein phosphatase.

43. The method of claim 26, comprising contacting the substrate with a detection module that binds to the substrate when the substrate is in the second state.

44. The method of claim 43, wherein the polypeptide substrate comprises a first polypeptide and the detection module comprises a second polypeptide.

45. The method of claim 44, wherein the second polypeptide comprises a 14-3-3 domain, an SH2 domain, a PTB domain, a chromodomain, a bromodomain, or an antibody.

46. The method of claim 26, wherein the conformational change that relieves quenching of the label by the quencher results in an increase in intensity of fluorescent emission from the label of at least about 10%.

47. The method of claim 26, wherein the sensor comprises one or more caging groups associated with the substrate, which caging groups inhibit the enzyme from acting upon the substrate, the method comprising uncaging the substrate, thereby freeing the substrate from inhibition by the one or more caging groups.

48. The method of claim 47, wherein uncaging the substrate comprises exposing the substrate to light of a first wavelength.

49. The method of claim 26, comprising contacting the enzyme with a test compound, assaying the activity of the enzyme in the presence of the test compound, and comparing the activity of the enzyme in the presence of the test compound with the activity of the enzyme in the absence of the test compound.

50. The method of claim 26, wherein contacting the enzyme and the sensor comprises introducing the sensor into a cell.

51. A composition comprising a labeled polypeptide, the labeled polypeptide comprising a fluorescent label, a polypeptide, and a quencher that is covalently connected to the polypeptide;
wherein the polypeptide comprises amino acid sequence X⁻⁴R⁻³R⁻²X⁻¹S⁰X⁺¹X⁺²;
where X⁻⁴ and X⁺² are independently selected from the group consisting of: an amino acid residue, an amino acid residue comprising the fluorescent label, and an amino acid residue comprising the quencher; and
where X⁻¹ and X⁺¹ are independently selected from the group consisting of: a hydrophobic amino acid residue, an amino acid residue comprising the fluorescent label, and an amino acid residue comprising the quencher.

52-57. (canceled)

58. A composition comprising:
a sensor for detecting an activity of a protein kinase, the sensor comprising
a) a substrate module comprising
i) a polypeptide substrate for the kinase, wherein the substrate is in a first, unphosphorylated state on which the kinase can act, thereby converting the substrate to a second, phosphorylated state,
ii) a fluorescent label, and
iii) a quencher, which quencher is covalently connected to the substrate; and
b) a detection module, which detection module binds to the substrate module when the substrate is in the second, phosphorylated state;
wherein binding of the detection module to the substrate module results in an increase in intensity of fluorescent emission from the label of at least about 1.5 fold.

59-73. (canceled)

74. A method of assaying activity of a protein kinase, the method comprising:
contacting the kinase with a sensor, the sensor comprising
a) a substrate module comprising
i) a polypeptide substrate for the kinase, wherein the substrate is in a first, unphosphorylated state on which the kinase can act, thereby converting the substrate to a second, phosphorylated state,
ii) a fluorescent label, and
iii) a quencher, which quencher is covalently connected to the substrate; and
b) a detection module, which detection module binds to the substrate module when the substrate is in the second, phosphorylated state;
wherein binding of the detection module to the substrate module results in an increase of at least about 1.5 fold in intensity of fluorescent emission from the label;
detecting the increase in intensity of fluorescent emission from the label; and
correlating the increase in intensity of fluorescent emission from the label to the activity of the kinase, thereby assaying the activity of the kinase.

75-89. (canceled)
